(19) United States
(12) Patent Application Publication
(10) Pub. No.: US 2024/0016735 A1
(43) Pub. Date: Jan. 18, 2024

(54) IONIC LIQUIDS FOR DRUG DELIVERY

(71) Applicant: PRESIDENT AND FELLOWS OF HARVARD COLLEGE, Cambridge, MA (US)

(72) Inventors: Samir MITRAGOTRI, Lexington, MA (US); Pavimol ANGSANTIKUL, Cambridge, MA (US); Abhirup MANDAL, Cambridge, MA (US); Eden E.L. TANNER, Cambridge, MA (US)

(73) Assignee: PRESIDENT AND FELLOWS OF HARVARD COLLEGE, Cambridge, MA (US)

(21) Appl. No.: 17/777,478

(22) PCT Filed: Nov. 19, 2020
(86) PCT No.: PCT/US2020/061185

§ 371 Date: May 17, 2022

Related U.S. Application Data
(60) Provisional application No. 62/939,088, filed on Nov. 22, 2019.

(51) Int. Cl. A61K 9/08; A61K 31/7088; A61K 47/18; A61K 47/12; A61K 47/20; A61K 47/24; A61P 17/06

(52) U.S. Cl. CPC A61K 9/08; A61K 31/7088; A61K 47/186; A61K 47/12; A61K 47/20; A61K 47/24; A61P 17/06; A61K 2039/505

(57) ABSTRACT

The technology described herein is directed to ionic liquids and methods of drug delivery.

### CROSS-REFERENCE TO RELATED APPLICATIONS

This application claims benefit under 35 U.S.C. § 119(e) of U.S. Provisional Application No. 62/939,088 filed Nov. 22, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 19, 2020, is named 002806-096230WOPT_SL.txt and is 25,930 bytes in size.

### TECHNICAL FIELD

The technology described herein relates to ionic liquids for stabilization and delivery of active compounds.

### BACKGROUND

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. These toxic and irritant effects are severe enough to mitigate any increase in the uptake or performance of the active compound.

### SUMMARY

As demonstrated herein, the inventors have identified characteristics of ionic liquids that provide surprising superior active compound uptake kinetics for certain types of active compounds. Accordingly, compositions and methods relating to these ionic liquids (ILs) with unexpectedly high efficacy are described herein.

In one aspect of any of the embodiments, described herein is a composition comprising at least one ionic liquid comprising: an anion which is at least one of: a) a carboxylic acid which is not a fatty acid; b) a carboxylic acid comprising an aliphatic chain of no more than 4 carbons; c) an aromatic anion; and/or d) an anion with a LogP of less than 1.0; and a cation comprising a quaternary ammonium.

In some embodiments of any of the aspects, the anion has a LogP of less than 1.0 and is: a) a carboxylic acid which is not a fatty acid; b) carboxylic acid comprising an aliphatic chain of no more than 4 carbons; or c) an aromatic anion. In some embodiments of any of the aspects, the fatty acid comprises an aliphatic chain of no more than 3 carbons. In some embodiments of any of the aspects, the anion comprises only one carboxylic acid group (e.g., R—COOH group). In some embodiments of any of the aspects, the anion is selected from the group consisting of: glycolic acid; propanoic acid; isobutryic acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid; isovaleric acid; hydrocinnaminic acid; 4-phenolsulfonic acid; phenyl phosphoric acid; and biphenyl-3-carboxylic acid.

In some embodiments of any of the aspects, the cation has a molar mass equal to or greater than choline. In some embodiments of any of the aspects, the quarternary ammonium has the structure of NR₄⁺ and at least one R group comprises a hydroxy group. In some embodiments of any of the aspects, the quarternary ammonium has the structure of NR₄⁺ and only one R group comprises a hydroxy group. In some embodiments of any of the aspects, the cation is C1, C6, or C7.

In some embodiments of any of the aspects, the ionic liquid comprises a ratio of cation to anion of from about 2:1 to about 1:1. In some embodiments of any of the aspects, the ionic liquid comprises a ratio of cation to anion of about 2:1. In some embodiments of any of the aspects, the ionic liquid has a cation:anion ratio of less than 1:1. In some embodiments of any of the aspects, the ionic liquid has a cation:anion ratio with an excess of cation.

In some embodiments of any of the aspects, the composition further comprises at least one active compound in combination with the at least one ionic liquid.

In some embodiments of any of the aspects, the active compound comprises a polypeptide. In some embodiments of any of the aspects, the polypeptide is an antibody or antibody reagent. In some embodiments of any of the aspects, the active compound has a molecular weight of greater than 450. In some embodiments of any of the aspects, the active compound has a molecular weight of greater than 500. In some embodiments of any of the aspects, the anion has a LogP of less than 1.0 and is: a) a carboxylic acid which is not a fatty acid; or b) a carboxylic acid comprising an aliphatic chain of no more than 4 carbons.

In some embodiments of any of the aspects, the active compound comprises a nucleic acid. In some embodiments of any of the aspects, the nucleic acid is an inhibitory nucleic acid. In some embodiments of any of the aspects, the nucleic acid is a siRNA. In some embodiments of any of the aspects, the anion has a LogP of less than 1.0 and is: a) a carboxylic acid which is not a fatty acid; or b) a carboxylic acid comprising an aliphatic chain of no more than 4 carbons; and/or c) an aromatic anion.

In some embodiments of any of the aspects, the ionic liquid is at a concentration of at least 0.1% w/v. In some embodiments of any of the aspects, the ionic liquid is at a concentration of from about 10 to about 70% w/v. In some embodiments of any of the aspects, the ionic liquid is at a concentration of from about 30 to about 50% w/v. In some embodiments of any of the aspects, the ionic liquid is at a concentration of from about 30 to about 40% w/v.

In some embodiments of any of the aspects, the composition is formulated for administration transdermally, to a mucus membrane, orally, subcutaneously, intradermally, parenterally, intratumorally, or intravenously. In some embodiments of any of the aspects, the composition is formulated for transdermal administration. In some embodiments of any of the aspects, the mucus membrane is nasal, oral, or vaginal.

In some embodiments of any of the aspects, the active compound is provided at a dosage of 1-40 mg/kg. In some embodiments of any of the aspects, the composition further comprises at least one non-ionic surfactant. In some embodiments of any of the aspects, the composition further comprises a pharmaceutically acceptable carrier. In some embodiments of any of the aspects, the composition is provided in a degradable capsule. In some embodiments of any of the aspects, the composition is an admixture. In some embodiments of any of the aspects, the composition is provided in one or more nanoparticles. In some embodiments of any of the aspects, the composition comprises one or more nanoparticles comprising the active compound, the nanoparticles in solution or suspension in a composition comprising the ionic liquid.

In one aspect of any of the embodiments, described herein is a method of administering at least one active compound, the method comprising administering a composition described herein. In some embodiments of any of the aspects, the composition is administered once. In some embodiments of any of the aspects, the composition is administered in multiple doses.

### BRIEF DESCRIPTION OF THE DRAWINGS

FIGS. 1A-1D. FIG. 1A depicts the chemical structures of choline and glycolic acid. CGLY variants (choline:glycolic acid molar ratios of 2:1, 1:1, and 1:2) were prepared by salt metathesis of choline bicarbonate and glycolic acid. FIG. 1B demonstrates the retained antigen binding capability of anti-human TNF-α mouse IgG1 antibody (clone MAb11) isolated from CGLY variants at concentration range between 20-90% v/v. FIG. 1C depicts the circular dichroism spectra of anti-human TNF-α IgG isolated from CGLY variants. The IgG was dispersed in 50% v/v CGLY variants and stored at RT (25° C.) for 1 h before dialyzed for 48 h. The beta-sheet secondary conformation of IgG was retained after exposed to CGLY solutions. FIG. 1D depicts SDS-PAGE of anti-human TNF-α IgG isolated from CGLY variants

FIGS. 2A-2B depict in vitro studies of CGLY variants on Caco-2 cell viability and IgG transport. FIG. 2A depicts caco-2 cell viability treated by CGLY variants. Data represented as mean±S.E. (n=6) FIG. 2B depicts the enhancement in FITC-IgG transport across Caco-2 monolayers in the presence of 30 mM CGLY variants. Data represented as mean±S.E.=5); (*p<0.05; CGLY_{2:1} treatment compared to CGLY_{1:1}, and CGLY 1:2). (^{##}p<0.01; all CGLY treatments compared to no CGLY treatment).

FIGS. 3A-3D depict in vitro molecular transport across Caco-2 cell monolayer with CGLY_{2:1} Enhancement in FITC-IgG (FIG. 3A) and Lucifer yellow (FIG. 3B) transport across Caco-2 monolayers in the presence of varied CGLY_{2:1} concentration. Data represented as mean±S.E. (n=FIG. 3C depicts the effect on tight junction integrity of Caco-2 cells upon treatment with various concentrations of CGLY. Data represented as mean±S.E. (n=5); (*p<0.05; **p<0.001; all CGLY_{2:1} treatment compared to no CGLY_{2:1} treatment). FIG. 3D depicts FITC-IgG transport across Caco-2 monolayers with 55 mM CGLY_{2:1} and with or without transcytosis inhibitors after 24 h incubation. Data represented as mean±S.E. (n=5)

FIG. 4A depicts the viscosity of porcine small intestinal mucus plotted as a function of shear rate range between 10-80 l/s, with 0%, 12.5% and 25% and 50% v/v CGLY_{2:1} in saline. The CGLY_{2:1} treatment was added to the mucus, followed by gently shaking then allowed to equilibrate for 30 mins before measurement. Data represented as mean (n=3). Black circles=0% v/v, dark grey circles=12.5% v/v, light grey circles 25% v/v, white circles 50% v/v. FIG. 4B depicts the mean viscosity values of porcine mucus at a shear rate of 49.87 l/s are shown with 0%, 12.5% and 25% and 50% v/v CGLY_{2:1} in saline. Data represented as mean±S.E (n=3); (*p<0.05, **p<0.01, ***p<0.001; CGLY_{2:1} treatment compared to no CGLY treatment).

FIGS. 5A-5C depict fluorescence microscopy images of intestinal villi after intrajejunal injection of FITC-IgG with CGLY_{2:1} (FIG. 5B), saline (FIG. 5C) and saline without FITC-IgG (FIG. 5A). Fluorescence microscopy imaging was performed in triplicate and a representative image was shown. Scale bars represent 200 μm. FIG. 5A depicts oral dose toxicity testing of CGLY 2:1. Oral gavage of CGLY_{2:1} (50% v/v) or saline, dosage 1250 mg/kg, n=2 was provided for 15 days. Outcomes were assessed by weight monitor, Blood chemistry, H&E staining of GI tract and major organs. FIG. 5D depicts fluorescence quantification of FITC-IgG per unit area on villi from FIGS. 5A-Data represented as mean±S.E. (n=10) FIG. 5E depicts in vivo plasma anti-human TNF-α IgG concentration after intrajejunal injection of the IgG in CGLY_{2:1} or saline, quantified by ELISA.

FIGS. 6A-6C depict in vivo toxicity study of CGLY_{2:1}. Rats were orally administered with CGLY_{2:1} or saline once daily for 7 consecutive days. FIG. 6A depicts rat body weight log from day 0 to day 7 during study. Data represented as mean±S.E. (n=6). FIG. 6B depicts the results when, on day 7, rats were sacrificed, and sections of the GI tract were processed for histological staining with hematoxylin and eosin (H&E). Scale bars represent 100 μm. FIG. 6C depicts a comprehensive metabolic panel of rats (n=6). The blood test conducted on day 7 did not reveal significant changes between the two groups, indicating normal liver and kidney functions after the CGLY administration. All bars and markers represent mean±S.E.

FIG. 7 depicts a diagram of drug delivery

FIG. 8 depicts functional antibody stability, as measured by ELISA, in the indicated ILs. As a general trend, small anions are more compatible with antibodies than larger anions.

FIG. 9 depicts functional antibody stability, as measured by size exclusion chromatography, in the indicated ILs. Antibody used was anti-human TNFα (mouse) (clone MAb11), with 2 day dialysis.

FIG. 10 depicts functional antibody stability, as measured by circular dichroism, in the indicated ILs. Antibody used was anti-human TNFα (mouse) (clone MAb11), with 2 day dialysis.

FIG. 11 depicts a graph of antibody concentration in serum after intrajejunal administration in the indicated compositions. Dosage was 200 μg/kg, n=3.

FIG. 12 depicts the experimental design for in vivo mAb local delivery.

FIG. 13 depicts the results of the in vivo mAb local delivery.

FIG. 14 depicts testing of CGLY 2:1 compatibility with other antibodies.

FIGS. 15 and 16 depict H&E staining of major organs (FIG. 15) and the GI tract (FIG. 16) in the toxicity test of FIG. 5A. Rats were orally administered with CGLY₂₁ or saline once daily for seven consecutive days. On day 7, rats were sacrificed and major organs including heart, liver, spleen, lung, and kidney were processed for histological staining with H&E. No differences were observed between CGLY_{2:1} and saline control groups. Scale bars represent 100 μm.

FIG. 17 depicts the structures of the ILs tested for siRNA delivery performance.

FIG. 18 depicts representative confocal micrograph images of transwell membranes covered with a layer of Caco-2 cells and incubated for 5 h with FITC-IgG dispersed in various concentrations of CGLY₂₁. Images were taken at 40× magnification. The images show DAPI labeled nuclei, FITC-IgG and an overlap of DAPI staining and FITC-IgG. Scale bars represent 50 μm.

FIGS. 19A-19E depict screening of cholinium-based bioactive IL-RNA complex for enhanced epidermal accumulation. (FIG. 19A) CD spectra of siRNA in phosphate-buffered saline (PBS) following incubation with IL (50% v/v) for 30 min and dialysis for 72 hours. (FIG. 19B) Representative native gel image of siRNA following IL incubation. bp, base pair. (FIG. 19C) Representative confocal images of siRNA (red) in the different skin layers (a) stratum corneum (SC), (b) epidermis, and (c) dermis, in the presence of IL combination (CAGE+CAPA) mixed at a ratio 1:1 following 24 hours of incubation. Left to right: Merged, Cy5, differential interference contrast (DIC). Scale bars, 50 μm. (FIGS. 19D and 19E) Transport of Cy5-labeled siRNA in the presence of individual ILs with concentration of 50% (v/v) (FIG. 19D) and combination of ILs at 50% (v/v) (FIG. 19E) into the different layers of skin determined by the tape-stripping method (n=3). Data are averages±SEM and were determined to be nonparametric by normality test and statistics by Kruskal-Wallis test for FIGS. 19D-19E. *P<0.05.

FIGS. 20A-20F depict a MD simulation that identifies the extent of IL-siRNA interaction for enhanced solvation and stability. (FIGS. 20A and 20B) Snapshot of simulation unit cell for CAGE and siRNA (FIG. 20A) and CAGE components found within 10 Å of siRNA (FIG. 20B) under periodic boundary conditions for 500 ns. (FIGS. 20C and 20D) Snapshot of simulation unit cell for the optimized IL combination (CAGE and CAPA, 1:1) and siRNA (FIG. 20C) and IL species found within 10 Å of siRNA (FIG. 20D) under similar conditions. (FIGS. 20E and 20F) Radius of gyration (RGYR) (FIG. 20E) and root mean square deviation (RMSD) (FIG. 20F) obtained over the course of 500 ns for CAPA and the IL combination (CAGE and CAPA) in contrast to CAGE (control).

FIGS. 21A-21E depict 3 MD simulations that establish enhanced lipid bilayer interactions and translocation mechanisms of the IL combination. (FIG. 21A) Lipid bilayer simulation with the aggregates of choline, geranic acid, and phenylpropanoic acid highlighted with a circle. (FIG. 21B) Enlarged view of the ionic species from the circle depicting closed interaction of ionic species with the phospholipid heads and tails. The aggregate contains all three ionic species contributing to the interaction with the lipid membrane. (FIG. 21C) Representative snapshot viewing perpendicular to membrane in the plane of the lipid bilayer. (FIGS. 21D and 21E) Average thickness of the lipid membrane (FIG. 21D) and average area per lipid (FIG. 21E) over the course of simulations in the presence of CAPA and the IL combination (CAGE and CAPA) in contrast to CAGE (control). All data are averages±SEM and were determined to be nonparametric by normality test and statistics by Kruskal-Wallis test for FIGS. 21D-21E. ****P<0.0001.

FIGS. 22A-22E demonstrate that IL-siRNA inhibits GAPDH expression following topical application without toxicity in mice. (FIG. 22A) Schematic illustration of the topical application schedule. (FIG. 22B) Representative histology [hematoxylin and eosin (H&E)] images of the skin tissue 5 days after topical application of IL-siRNA. Scale bars, 100 μm; magnification, ×10. (FIG. 22C) Confocal images of epidermal accumulation of Cy5-siRNA with and without IL in a mouse skin tissue. Scale bars, 50 μm (FIG. 22D) GAPDH mRNA expression was measured by qPCR. β-Actin mRNA expression was used for normalization. Data are averages±SEM and were determined to be nonparametric by normality test and statistics by Kruskal-Wallis test. *P<0.05, ***P<0.001, and ****P<0.0001. (FIG. 22E) GAPDH levels in the skin samples were determined using a GAPDH enzyme-linked immunosorbent assay. Data are averages±SEM, statistics by one-way ANOVA with Tukey HSD posttest. ****P<0.0001 (control, n=5; naked siRNA, n=5; IL-siCon, n=4; IL-siRNA, n=8).

FIGS. 23A-23J demonstrate that local inhibition of NFKBIZ by topical IL-siRNA suppresses imiquimod-induced psoriasis-like skin inflammation and other key psoriasis-associated genes. (FIG. 23A) Schematic illustration of the application schedule for disease induction and IL-siRNA topical administration. (FIG. 23B) Psoriasis-induced mice were treated topically with IL-NFKBIZ siRNA and were compared with untreated and IL-applied groups. (FIG. 23C) H&E staining of the psoriasis-induced skin sections from the mice with and without treatment. Scale bars, 50 μm; magnification, ×10. (FIG. 23D) Skin sections from the mice were analyzed for keratinocyte proliferation (proliferation marker, Ki67) by IHC. Scale bars, 100 μm. (FIGS. 23E and 23F) Erythema and scaling scores obtained by blindly scoring using the human PASI scoring system daily on a scale from 0 (no alteration) to 4 (very distinct alteration). (FIG. 23G) Heat map for the expression levels of various psoriasis-associated genes following treatment with IL-NFKBIZ siRNA in comparison with the untreated (control) and IL-siCon-treated groups. (FIGS. 23H to 23J) mRNA expression levels were measured by qPCR, and β-actin mRNA expression was used for normalization for NFKBIZ, TNF-α, and IL-17A, respectively. Data are averages±SEM, statistics by one-way ANOVA with Tukey HSD posttest. *P<0.05, **P<0.01, and ****P<0.0001 (control, n=4; IL, n=4; IL-siCon, n=4; IL-siRNA, n=8).

FIGS. 24A-24E depict the design and synthesis of an in-house cholinium-based IL library for improved biocompatibility and interaction with RNA. (FIG. 24A) Cholinium-based IL library constituting various anions that are synthesized with CAGE as the reference IL. (FIG. 24B) General synthetic scheme for salt metathesis employed in the synthesis of ILs. (FIG. 24C) Synthetic scheme of the optimized IL combination (CAGE+CAPA) for the delivery of siRNA. (FIG. 24D) 1H-NMR spectra of the synthesized ILs that remain viscous at RT, (a) CAGE, (b) CAVA, (c) CAPA and (d) CADA. (FIG. 24E) Relative density of the siRNA band following IL incubation measured with Image J software.

FIGS. 25A-25D demonstrate improved epidermal accumulation of Cy5-labelled siRNA in presence of ILs. (FIG. 25A) Schematic representation of the Franz diffusion cell (FDC) setup for the ex-vivo porcine skin permeation studies. (FIG. 25B) Representative confocal images of the controls, naked siRNA and siRNA in presence of CAGE. (FIG. 25C) Epidermal accumulation of Cy5-siRNA in the presence of newly synthesized cholinium-based ILs and combinations at a ratio 1:1 following 24 hrs incubation of porcine skin. Left to right: merged, Cy5, differential interference contrast (DIC). Scale bars, 50 μm. (FIG. 25D) Transport of Cy5-labelled siRNA into the different layers of skin determined by tape-stripping method (n=3). Data are averages±SEM, was determined to be non-parametric by normality test and statistics by Kruskal-Wallis test.

FIGS. 26A-26B depict the major contribution of IL species mobility in IL-lipid bilayer interaction and permeation. (FIG. 26A) Lipid bilayer simulation in presence of the IL combination (highlighted with the circle) (FIG. 26B) Trajectories of the individual ionic species within the IL combination, CAGE+CAPA simulation using the python library MDAnalysis.

FIGS. 27A-27D depict the highly biocompatible IL formulation without toxicity and irritation upon topical application. (FIG. 27A) Application sites of healthy mice treated topically with IL-GAPDH siRNA and were compared with water- and IL-siCon groups. (FIG. 27B) H&E staining of the skin section from the healthy mice treated topically with IL-siCon for 4 consecutive days. Scale bars, 100 μm, Magnification, 10×. (FIG. 27C) Skin sections from the healthy mice were analyzed for hyper-proliferation by staining with the proliferation marker, Ki67. Scale bars, 100 μm. Quantitative analysis for IHC was not performed since no proliferated regions were observed. (FIG. 27D) TNF-α mRNA expression was measured by qPCR and β-actin mRNA expression was used for normalization. Data are averages±SEM, statistics by one-way ANOVA with Tukey HSD post-test. *P<0.05, **P<0.01, ****P<0.0001. (control, n=5; naked siRNA, n=5; IL-siCon, n=4; IL-siRNA, n=8).

FIGS. 28A-28D depict the characterization of IL-siCon effects in imiquimod-induced psoriatic mice. (FIG. 28A) Psoriasis-induced mice were treated topically with IL-siCon. For 4 consecutive days. (FIG. 28B) H&E staining of the skin section from the imiquimod-induced psoriatic mice treated topically with IL-siCon. Scale bars, 50 μm, Magnification, 10×. (FIG. 28C) Skin sections from the psoriatic mice were analyzed for hyper-proliferation by staining with the proliferation marker, Ki67. Scale bars, 100 μm. (FIG. 28DD) Epidermal thickness; means of epidermal thickness calculated based on 10-15 random site measurements with Image J software. Data are averages±SEM, statistics by one-way ANOVA with Tukey HSD post-test. *P<0.05, ****P<0.0001.

FIGS. 29A-29C depict the effects of IL-NFKBIZ siRNA in imiquimod-induced psoriasis-like skin inflammation in mice. Imiquimod-induced psoriatic mice were analyzed for cumulative score (FIG. 29A), body weight (FIG. 29B) and skin thickness (FIG. 29C), monitored by the double skin-fold thickness (DSFT) over a period of 5 days of induction/application. Data are averages±SEM. (control, n=4; IL, n=4; IL-siRNA, n=8).

FIGS. 30A-30J depict the downstream effect of silencing NFKBIZ on psoriasis-related gene products. mRNA expression was measured by qPCR and β-actin mRNA expression was used for normalization for cytokines, IL-17C, IL-19, IL-22, IL-23A, IL-36A, IL-36G (FIGS. 30A-30F); chemokine, CCL 20 (FIG. 30G); S100 protein, S100A9 (FIG. 30H); antimicrobial protein, lipocalin-2, LCN2 and β-defensin-2, DEFB4 (FIG. 30J). Data are averages±SEM, statistics by one-way ANOVA with Tukey HSD post-test. *P<0.05, **P<0.01, ***P<0.001, ****P<0.0001. (control, n=4; IL, n=4; IL-siCon, n=4; IL-siRNA, n=8).

### DETAILED DESCRIPTION

The data provided herein demonstrate that the anion of an ionic liquid (IL) exerts the predominant influence on whether particular active agents will be transported across a biological barrier (e.g., an epithelial layer, such as the dermis). Anions with low hydrophobicity and/or an aromatic group provide improved drug delivery characteristics for antibody and siRNA cargo molecules than anions in previously described ILs such as CAGE (choline and geranic acid). In selecting a cation to pair with the anion, the primary concern is that the cation not associate too closely with the anion—close association causes the anion to be retained on the initial side of the biological barrier.

Accordingly, in one aspect of any of the embodiments, described herein is a composition comprising at least one ionic liquid comprising 1) an anion which is at least one of:
a) a carboxylic acid which is not a fatty acid;
b) a carboxylic acid comprising an aliphatic chain of no more than 4 carbons;
c) an aromatic anion; and/or
d) an anion with a LogP of less than 1.0; and
   2) a cation comprising a quaternary ammonium.

In one aspect of any of the embodiments, described herein is a composition comprising at least one ionic liquid comprising 1) an anion which is a carboxylic acid as described herein; and 2) a cation comprising a quaternary ammonium.

The term “ionic liquids (ILs)” as used herein refers to organic salts or mixtures of organic salts which are in liquid state at room temperature. This class of solvents has been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group), examples include but are not limited to alcohols, fatty acids, and amines. The at least one anionic and at least one cationic component may be present in any molar ratio. Exemplary molar ratios (cation:anion) include but are not limited to 1:1, 1:2, 2:1, 1:3, 3:1, 2:3, 3:2, and ranges between these ratios. For further discussion of ionic liquids, see, e.g., Hough, et ah, “The third evolution of ionic liquids: active pharmaceutical ingredients”, New Journal of Chemistry, 31: 1429 (2007) and Xu, et al., “Ionic Liquids: Ion Mobilities, Glass Temperatures, and Fragilities”, Journal of Physical Chemistry B, 107(25): 6170-6178 (2003); each of which is incorporated by reference herein in its entirety. In some embodiments of any of the aspects, the ionic liquid or solvent exists as a liquid below 100° C. In some embodiments of any of the aspects, the ionic liquid or solvent exists as a liquid at room temperature.

As demonstrated herein, anions with low hydrophobicity, relatively short carbon chains, and/or an aromatic group provide improved drug delivery characteristics for large polypeptide (e.g., antibody) or nucleic acid cargo molecules. In some embodiments, improved drug delivery characteristics comprise reduced denaturation or degradation of the cargo molecule. In some embodiments, improved drug delivery characteristics comprise increased ability to cross biological barriers (e.g., increased permeability). In some embodiments of any of the aspects, an anion with low hydrophobicity and/or relatively short carbon chain provides improved drug delivery characteristics for large polypeptide (e.g., antibody) cargo molecules. In some embodiments of any of the aspects, an anion with aromatic group(s) and/or relatively short carbon chain provides improved drug delivery characteristics for nucleic acid cargo molecules.

In some embodiments of any of the aspects, the anion of an IL described herein is hydrophobic.

In some embodiments of any of the aspects, the anion of an IL described herein comprises a carboxylic acid. In some embodiments of any of the aspects, the anion of an IL described herein comprises a carboxylic acid which is not a fatty acid.

A carboxylic acid is a compound having the structure of Formula I, wherein R can be any group.

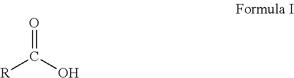

Generally, the anion is R—X⁻, where X is CO₂⁻, SO₃⁻, OSO₃²⁻ or OPO₃²⁻; and R is optionally substituted C₁-C₁₀alkyl, optionally substituted C₂-C₁₀alkenyl, or optionally substituted C₂-C₁₀alkynyl, optionally substituted aryl, or optionally substituted heteroaryl.

In some embodiments, R is an optionally substituted linear or branched C₁-C₉alkyl. For example, R is a C₁-C₉alkyl optionally substituted with 1, 2, 3, 4, 5 or 6 substituents independently selected from the group consisting of C₁-C₃alkyl, hydroxy (OH), halogen, oxo (═O), carboxy (CO₂), cyano (CN) and aryl. In some embodiments, R is a C₁-C₆alkyl optionally substituted with 1, 2, 3, 4 or 5 substituents independently selected from the group consisting of C₁-C₃alkyl, hydroxy, carboxy and phenyl. Preferably, R is a C₁-C₅alkyl, optionally substituted with 1, 2, 3, 4 or 5 substituents independently selected from the group consisting of methyl, ethyl, hydroxyl, carboxy, and phenyl. Exemplary alkyls for R include, but are not limited to, methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl and nonyl.

In some embodiments, R is an optionally substituted linear or branched C₂-C₅alkenyl. For example, R is a C₂-C₉alkenyl optionally substituted with 1, 2, 3, 4, 5 or 6 substituents independently selected from the group consisting of C₁-C₃alkyl, hydroxy, halogen, oxo, carboxy, cyano and aryl. In some embodiments, R is a C₂-C₅alkenyl optionally substituted with 1, 2, 3, 4 or 5 substituents independently selected from the group consisting of C₁-C₃alkyl, hydroxy, carboxy and phenyl. Preferably, R is a C₁-C₅alkenyl, optionally substituted with 1, 2, 3, 4 or 5 substituents independently selected from the group consisting of methyl, ethyl, hydroxyl, carboxy, and phenyl. Exemplary alkenyls for R include, but are not limited to, ethenyl, 2-carboxyethenyl, 1-methylpropenyl and 2-methylpropenyl.

In some embodiments, R is an optionally substituted aryl or heteroaryl. For example, R is an aryl or heteroaryl optionally substituted with 1, 2, 3, 4, 5 or 6 substituents independently selected from the group consisting of C₁-C₃alkyl, hydroxy, halogen, oxo, carboxy, cyano and aryl. In some embodiments, R is an aryl optionally substituted with 1, 2, 3, 4 or 5 substituents independently selected from the group consisting of C₁-C₃alkyl, hydroxy, carboxy and phenyl. Preferably R is a phenyl substituted with 1, 2 or 3 substituents independently selected from the group consisting of methyl, ethyl, hydroxyl, carboxy, and phenyl. Exemplary aryls for R include, but are not limited to, phenyl, 2-hydroxyphenyl, 3-hydroxyphenyl, 4-hydroxyphenyl, dihydroxyphenyl, trihydroxyphenyl, 3,4,5-trihydroxyphenyl, and 1,1-biphen-4-yl.

In some embodiments, X is CO₂⁻ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In some other embodiments, X is OSO₃⁻ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In yet some other embodiments, X is OPO₃²⁻ or SO₃⁻ and R is 2-hydroxyphenyl, 3-hydroxyphenyl or 4-hydroxyphenyl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). An alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An “alkenyl” is an unsaturated alkyl group is one having one or more double bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), and the higher homologs and isomers.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Exemplary aryl and heteroaryl groups include, but are not limited to, phenyl, 4-nitrophenyl, 1-naphthyl, 2-naphthyl, biphenyl, 4-biphenyl, pyrrole, 1-pyrrolyl, 2-pyrrolyl, 3-pyrrolyl, pyrazole, 3-pyrazolyl, imidazole, imidazolyl, 2-imidazolyl, 4-imidazolyl, benzimidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, thiazolyl, 2-thiazolyl, 4-thiazolyl, 5-thiazolyl, 2-furyl, 3-furyl, 2-thienyl, 3-thienyl, pyridine, 2-pyridyl, naphthyridinyl, 3-pyridyl, 4-pyridyl, benzophenonepyridyl, pyridazinyl, pyrazinyl, 2-pyrimidyl, 4-pyrimidyl, pyrimidinyl, 5-benzothiazolyl, purinyl, 2-benzimidazolyl, indolyl, 5-indolyl, quinoline, quinolinyl, 1-isoquinolyl, 5-isoquinolyl, 2-quinoxalinyl, 5-quinoxalinyl, 3-quinolyl, 6-quinolyl, furan, furyl or furanyl, thiophene, thiophenyl or thienyl, diphenylether, diphenylamine, and the like.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used herein, “fatty acid” refers to a carboxylic acid wherein R comprises a saturated or unsaturated aliphatic chain, e.g., R has the formula CₙH₂ₙ₊₁. In some embodiments of any of the aspects, the fatty acid is a monocarboxylic acid. The fatty acid can be natural or synthetic. The aliphatic chain of the fatty acid can be saturated, unsaturated, branched, straight, and/or cyclic. In some embodiments of any of the aspects, the aliphatic chain does not comprise an aromatic group. In some embodiments of any of the aspects, the aliphatic chain comprises, consists of, or consists essentially of an alkyl or alkene chain.

Exemplary carboxylic acids which are not fatty acids can include, but are not limited to lactic acid; glycolic acid; malonic acid; maleic acid; glutaric acid; citric acid; gluconic acid; and adipic acid.

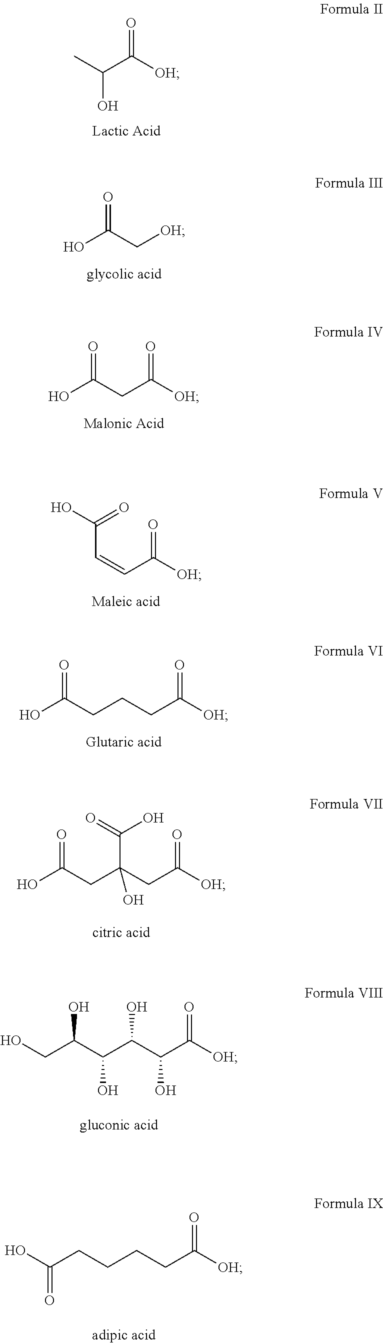

In some embodiments, the carboxylic acid which is not a fatty acid comprises no more than 5 carbons in the R group, either in a straight or branched configuration. In some embodiments, the carboxylic acid which is not a fatty acid comprises a hydroxy group in the R group. In some embodiments, the carboxylic acid which is not a fatty acid comprises one or more carboxylic acids in the R group.

In some embodiments, the carboxylic acid which is not a fatty acid comprises no more than 5 carbons in the R group, either in a straight or branched configuration, and comprises a hydroxy group in the R group. In some embodiments, the carboxylic acid which is not a fatty acid comprises 1-5 carbons in the R group, either in a straight or branched configuration, and comprises a hydroxy group in the R group.

In some embodiments, the carboxylic acid which is not a fatty acid comprises no more than 5 carbons in the R group, either in a straight or branched configuration, and comprises one or more carboxylic acid groups in the R group. In some embodiments, the carboxylic acid which is not a fatty acid comprises 1-5 carbons in the R group, either in a straight or branched configuration, and comprises one or more carboxylic acid groups in the R group.

In some embodiments, the carboxylic acid which is not a fatty acid comprises 1-5 carbons in the R group, either in a straight or branched configuration, and comprises one carboxylic acid group in the R group.

When the number of carbons in a chain is referred to herein, it is contemplated that the entire number of carbons in the chain (including branches) is referred to. In the case of a straight chain, this is the same as the carbon chain length. In the case of a branched chain, “chain length” refers to the longest carbon chain branch of the branched chain.

In some embodiments, the anion comprises one carboxylic acid group.

Exemplary carboxylic acids comprising an aliphatic chain of no more than 4 carbons can include propanoic acid (a fatty acid); isobutryic acid (a fatty acid); butyric acid (a fatty acid), 3,3-dimethylacrylic acid (a fatty acid); dimethylacrylic acid (a fatty acid); and isovaleric acid (a fatty acid).

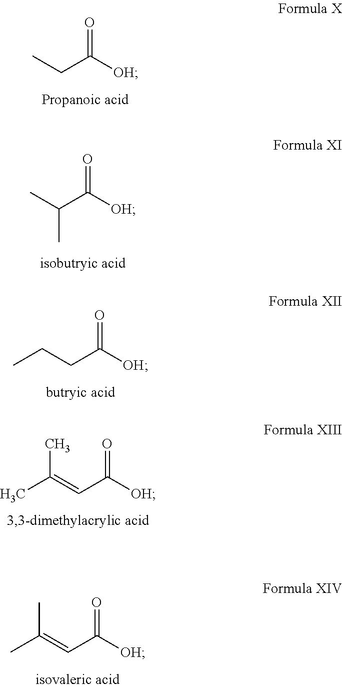

Exemplary alternative anions contemplated herein include decanoic acid and ethylhexyl sulfate.

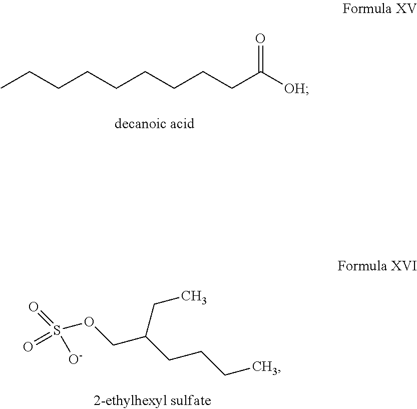

Exemplary aromatic anions include but are not limited to gallic acid, hydrocinnamic acid, hydroxybenzenesulfonic acid, 4-hydroxybenzenesulfonic acid (4-phenolsulfonic acid), biphenyl-3-carboxylic acid, and phenyl phosphoric acid.

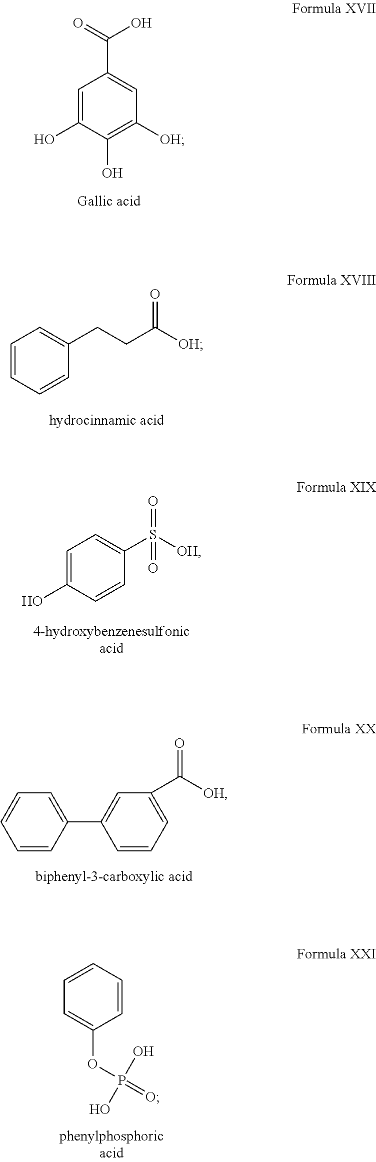 [SMILES: O=C(O)c1cc(O)c(O)c(O)c1 ; O=C(O)CCc1ccccc1 ; O=S(=O)(O)c1ccc(O)cc1 ; O=C(O)c1cccc(-c2ccccc2)c1 ; O=P(O)(O)Oc1ccccc1]

Hydrophobicity may be assessed by analysis of logP. “LogP” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.

Partition Coefficient, P=[Organic]/[Aqueous] where [ ]=concentration

Log P=log₁₀(Partition Coefficient)=log₁₀P

In practice, the LogP value will vary according to the conditions under which it is measured and the choice of partitioning solvent. A LogP value of 1 means that the concentration of the compound is ten times greater in the organic phase than in the aqueous phase. The increase in a logP value of 1 indicates a ten fold increase in the concentration of the compound in the organic phase as compared to the aqueous phase.

In some embodiments of any of the aspects, the anion has a LogP of less than 1.0. In some embodiments of any of the aspects, the anion has a LogP of less than 0.80. In some embodiments of any of the aspects, the anion has a LogP of less than 0.75. In some embodiments of any of the aspects, the anion has a LogP of less than 0.50. In some embodiments of any of the aspects, the anion has a LogP of less than 0.25. In some embodiments of any of the aspects, the anion has a LogP of less than 0.

In one aspect of any of the embodiments, described herein is a composition comprising at least one ionic liquid comprising 1) an anion with a LogP of less than 1.0 and which is a carboxylic acid which is not a fatty acid, and 2) a cation comprising a quaternary ammonium. In one aspect of any of the embodiments, described herein is a composition comprising at least one ionic liquid comprising 1) an anion with a LogP of less than 1.0 and which is a carboxylic acid comprising an aliphatic chain of no more than 4 carbons, and 2) a cation comprising a quaternary ammonium. In one aspect of any of the embodiments, described herein is a composition comprising at least one ionic liquid comprising 1) an anion with a LogP of less than 1.0 and which is aromatic, and 2) a cation comprising a quaternary ammonium.

In some embodiments of any of the aspects, the anion of an IL described herein has a pKa of less than 4.0. In some embodiments of any of the aspects, the anion of an IL described herein has a pKa of less than 4.0 and aa LogP of less than 1.0.

The pKa and LogP values for anions are known in the art and/or can be calculated by one of skill in the art. For example, PubChem and SpiderChem provide these values for various anions and chemical manufacturers typically provide them as part of the catalog listings for their products. pKa and LogP values for exemplary anions are provided in Table 1 herein.

Exemplary, non-limiting anions are provided in Table 1 below.

TABLE 1

| | LogP | pKa |
|---|---|---|
| Glycolic acid | −1.11 | 3.8 |
| Propanoic acid | 0.33 | 4.88 |
| Isoburtyric acid | 0.94 | 4.84 |
| Butyric acid | 0.79 | 4.82 |
| Gallic acid | 0.70 | 4.40 |
| Lactic acid | −0.72 | 3.86 |
| Malonic acid | −0.81 | 2.8 |
| Decanoic Acid | 4.09 | 4.9 |
| Maleic acid | −0.48 | 1.83 |
| Glutaric acid | −0.29 | 4.34 |
| Citric acid | −1.64 | 2.79 |
| 3,3-dimethylacrylic acid | 1.2 | 5.02 |
| Gluconic acid | −3.4 | 3.39 |
| Adipic acid | 0.08 | 4.4 |
| 2-Ethylhexyl sulfate | 3.10 | |
| 4-hydroxybenzenesulfonic acid | 0.2 | 9.11 |
| Isovaleric acid | 1.16 | 4.77 |
| Hydrocinnamic acid | 1.84 | 4.66 |
| Phenylphosphoric acid | 1.05 | 9.99 |
| Bipheny1-3-carboxylic acid | 3.5 | 4.14 |

In some embodiments of any of the aspects, the anion is an alkane. In some embodiments of any of the aspects, the anion is an alkene. In some embodiments of any of the aspects, the anion comprises a single carboxyl group. In some embodiments of any of the aspects, the carbon chain of the carboxylic acid comprises one or more substituent groups. In some embodiments of any of the aspects, the carbon chain backbone of the carboxylic acid comprises one or more substituent groups, wherein each substituent group comprises at least one carbon atom. In some embodiments of any of the aspects, the carbon chain backbone of the carboxylic acid comprises one or more substituent groups, wherein at least one substituent group comprises a methyl group. In some embodiments of any of the aspects, the carbon chain backbone of the carboxylic acid comprises two substituent groups, wherein each substituent group comprises at least one carbon atom. In some embodiments of any of the aspects, the carbon chain backbone of the carboxylic acid comprises two substituent groups, wherein one substituent group comprises a methyl group. In some embodiments of any of the aspects, the carbon chain backbone of the carboxylic acid comprises two substituent groups, wherein each substituent group comprises a methyl group.

In some embodiments of any of the aspects, the anion is an unsubstituted alkane. In some embodiments of any of the aspects, the anion is an unsubstituted alkene. In some embodiments of any of the aspects, the carbon chain backbone of the carboxylic acid comprises one or more substituent groups. In some embodiments of any of the aspects, the carbon chain of the carboxylic acid comprises one or more substituent groups, wherein each substituent group comprises at least one carbon atom. In some embodiments of any of the aspects, the carbon chain of the carboxylic acid comprises one or more substituent groups, wherein each substituent group is alkyl, aryl, heteroalkayl, heteroaryl, alkane, or alkene. In some embodiments of any of the aspects, the carbon chain of the carboxylic acid comprises one or more substituent groups, wherein each substituent group is unsubstituted alkyl, unsubstituted aryl, unsubstituted heteroalkayl, unsubstituted heteroaryl, unsubstituted alkane, or unsubstituted alkene.

As described herein, in selecting a cation to pair with the anion, the primary concern is that the cation not associate too closely with the anion—close association causes the anion to be retained on the initial side of the biological barrier. Choline and derivatives thereof are shown to be particularly well suited as IL cations for the types of anions described herein. Accordingly, the cation of an IL described herein can be a cation comprising a quaternary ammonium. A quarternary ammonion is a positively charged polyatomic ion of the structure NR₄⁺, each R independently being an alkyl group or an aryl group.

The general term “quaternary ammonium” relates to any compound that can be regarded as derived from ammonium hydroxide or an ammonium salt by replacement of all four hydrogen atoms of the NH₄⁺ ion by organic groups. For example, the quaternary ammonium has the structure of NR₄⁺, where each R is independently selected from hydroxyl, optionally substituted C₁-C₁₀alkyl, optionally substituted C₂-C₁₀alkenyl, optionally substituted C₂-C₁₀alkynyl, optionally substituted aryl, or optionally substituted heteroaryl.

In some embodiments of any of the aspects, the cation has a molar mass equal to or greater than choline, e.g., a molar mass equal to or greater than 104.1708 g/mol. In some embodiments of any of the aspects, the cation has a molar mass greater than choline, e.g., a molar mass equal greater than 104.1708 g/mol.

In some embodiments of any of the aspects, each R group of the quaternary ammonium independently comprises an alkyl, alkane, alkene, or aryl. In some embodiments of any of the aspects, each R group of the quaternary ammonium independently comprises an alkyl, alkane, or alkene. In some embodiments of any of the aspects, each R group of the quaternary ammonium independently comprises an alkane or alkene. In some embodiments of any of the aspects, each R group of the quaternary ammonium independently comprises a carbon chain of no more than 10 carbon atoms in length, e.g., no more than 10, 11, 12, 13, 14, 15, 16, 17, 18, 19, 20, 25, or 30 carbon atoms in length. In some embodiments of any of the aspects, each R group of the quaternary ammonium independently comprises a carbon chain of no more than 12 carbon atoms in length. In some embodiments of any of the aspects, each R group of the quaternary ammonium independently comprises a carbon chain of no more than 15 carbon atoms in length. In some embodiments of any of the aspects, each R group of the quaternary ammonium independently comprises a carbon chain of no more than 20 carbon atoms in length.

In some embodiments of any of the aspects, each R group of the quaternary ammonium independently comprises a carbon chain of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternary ammonium independently comprises a carbon chain of no more than 12 carbon atoms. In some embodiments of any of the aspects, each R group of the quaternary ammonium independently comprises a carbon chain of no more than 15 carbon atoms. In some embodiments of any of the aspects, each R group of the quaternary ammonium independently comprises a carbon chain of no more than 20 carbon atoms.

In some embodiments of any of the aspects, each R group of the quaternary ammonium independently comprises an alkyl group of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternary ammonium independently comprises an alkyl group of no more than 12 carbon atoms. In some embodiments of any of the aspects, each R group of the quaternary ammonium independently comprises an alkyl group of no more than 15 carbon atoms. In some embodiments of any of the aspects, each R group of the quaternary ammonium independently comprises an alkyl group of no more than 20 carbon atoms.

In some embodiments of any of the aspects, each R group of the quaternary ammonium independently comprises an alkane, alkene, aryl, heteroaryl, alkyl, or heteroalkyl. In some embodiments of any of the aspects, each R group of the quaternary ammonium independently comprises an unsubstituted alkane, unsubstituted alkene, unsubstituted aryl, unsubstituted heteroaryl, unsubstituted alkyl, or unsubstituted heteroalkyl. In some embodiments of any of the aspects, each R group of the quaternary ammonium independently comprises an unsubstituted alkane. In some embodiments of any of the aspects, each R group of the quaternary ammonium independently comprises an unsubstituted alkene. In some embodiments of any of the aspects, each R group of the quaternary ammonium independently comprises one or more substituent groups.

In some embodiments of any of the aspects, at least one R group of the quaternary ammonium comprises a hydroxy group. In some embodiments of any of the aspects, one R group of the quaternary ammonium comprises a hydroxy group. In some embodiments of any of the aspects, only one R group of the quaternary ammonium comprises a hydroxy group.

Exemplary, non-limiting cations can include choline and any of the cations designated C1-C7 which are defined by structure below.

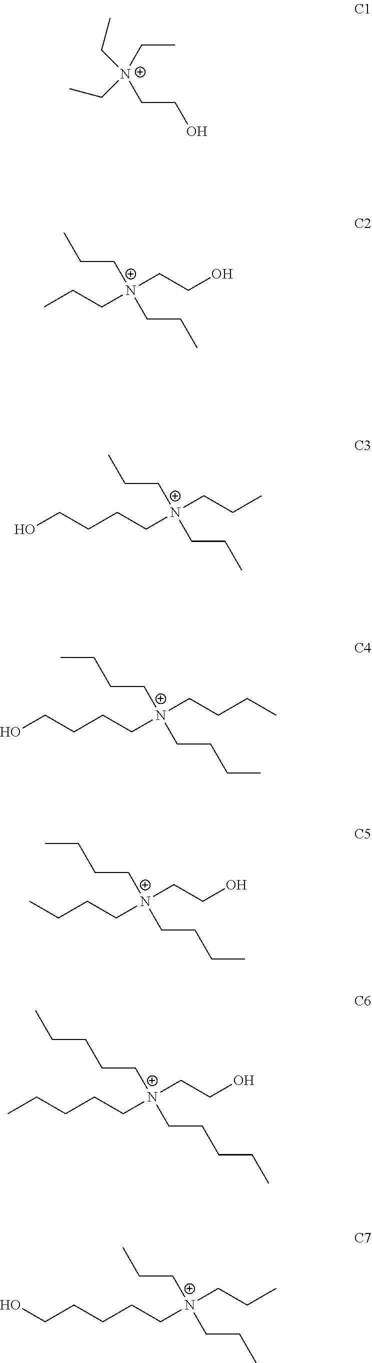

Further non-limiting examples of cations include the following:
1-(hydroxymethyl)-1-methylpyrrolidin-1-ium
1-(2-hydroxyethyl)-1-methylpyrrolidin-1-ium
1-ethyl-1-(3-hydroxypropyl)pyrrolidin-1-ium
1-(3-hydroxypropyl)-1-methylpyrrolidin-1-ium
1-(4-hydroxybutyl)-1-methylpyrrolidin-1-ium
1-ethyl-1-(4-hydroxybutyl)pyrrolidin-1-ium
1-(4-hydroxybutyl)-1-propylpyrrolidin-1-ium
1-(5-hydroxypentyl)-1-propylpyrrolidin-1-ium
1-ethyl-1-(5-hydroxypentyl)pyrrolidin-1-ium
1-(5-hydroxypentyl)-1-methylpyrrolidin-1-ium
1-(hydroxymethyl)-1-methylpiperidin-1-ium
1-(2-hydroxyethyl)-1-methylpiperidin-1-ium
1-ethyl-1-(2-hydroxyethyl)piperidin-1-ium
1-ethyl-1-(3-hydroxypropyl)piperidin-1-ium
1-(3-hydroxypropyl)-1-propylpiperidin-1-ium
1-(3-hydroxypropyl)-1-methylpiperidin-1-ium
1-(4-hydroxybutyl)-1-methylpiperidin-1-ium
1-ethyl-1-(4-hydroxybutyl)piperidin-1-ium
1-(4-hydroxybutyl)-1-propylpiperidin-1-ium
1-butyl-1-(5-hydroxypentyl)piperidin-1-ium
1-(5-hydroxypentyl)-1-propylpiperidin-1-ium
1-ethyl-1-(5-hydroxypentyl)piperidin-1-ium
1-(5-hydroxypentyl)-1-methylpiperidin-1-ium
3-ethyl-1-methyl-1H-imidazol-3-ium
1-methyl-3-propyl-1H-imidazol-3-ium
3-butyl-1-methyl-1H-imidazol-3-ium
1-methyl-3-pentyl-1H-imidazol-3-ium
1,2-dimethyl-3-pentyl-1H-imidazol-3-ium
3-butyl-1,2-dimethyl-1H-imidazol-3-ium
1,2-dimethyl-3-propyl-1H-imidazol-3-ium
3-(hydroxymethyl)-1,2-dimethyl-1H-imidazol-3-ium
3-(2-hydroxyethyl)-1,2-dimethyl-1H-imidazol-3-ium
3-(3-hydroxypropyl)-1,2-dimethyl-1H-imidazol-3-ium
3-(4-hydroxybutyl)-1,2-dimethyl-1H-imidazol-3-ium
3-(5-hydroxypentyl)-1,2-dimethyl-1H-imidazol-3-ium
3-(5-hydroxypentyl)-1-methyl-1H-imidazol-3-ium
3-(4-hydroxybutyl)-1-methyl-1H-imidazol-3-ium
3-(3-hydroxypropyl)-1-methyl-1H-imidazol-3-ium
3-(2-hydroxyethyl)-1-methyl-1H-imidazol-3-ium
3-(hydroxymethyl)-1,2,4,5-tetramethyl-1H-imidazol-3-ium
3-(2-hydroxyethyl)-1,2,4,5-tetramethyl-1H-imidazol-3-ium
3-(3-hydroxypropyl)-1,2,4,5-tetramethyl-1H-imidazol-3-ium
3-(4-hydroxybutyl)-1,2,4,5-tetramethyl-1H-imidazol-3-ium
3-(5-hydroxypentyl)-1,2,4,5-tetramethyl-1H-imidazol-3-ium
1-(5-hydroxypentyl)pyridin-1-ium
1-(4-hydroxybutyl)pyridin-1-ium
1-(3-hydroxypropyl)pyridin-1-ium
1-(2-hydroxyethyl)pyridin-1-ium
1-(hydroxymethyl)pyridin-1-ium
1-hydroxypyridin-1-ium
(hydroxymethyl)trimethylphosphonium
triethyl(hydroxymethyl)phosphonium
triethyl(2-hydroxyethyl)phosphonium
(2-hydroxyethyl)tripropylphosphonium
(3-hydroxypropyl)tripropylphosphonium
tributyl(3-hydroxypropyl)phosphonium
(3-hydroxypropyl)tripentylphosphonium
(4-hydroxybutyl)tripentylphosphonium
(5-hydroxypentyl)tripentylphosphonium

In some embodiments of any of the aspects, the cation is choline, C1, C6, and/or C7. In some embodiments of any of the aspects, the cation is C1, C6, and/or C7.

In some embodiments of any of the aspects, the cation is choline, C1, C6, and/or C7 and the anion is an anion selected from Table 1. In some embodiments of any of the aspects, the cation is choline and the anion is an anion selected from Table 1.

Non-limiting, exemplary combinations of cation and anions are provided in Table 2 below.

TABLE 2

| | Choline | C1 | C2 | C3 | C4 | C5 | C6 | C7 |
|---|---|---|---|---|---|---|---|---|
| Glycolic acid | x | x | x | x | x | x | x | x |
| Propanoic acid | x | X | X | X | X | X | X | X |
| Isoburtyric acid | X | X | X | X | X | X | X | X |
| Butyric acid | X | X | X | X | X | X | X | X |
| Gallic acid | X | X | X | X | X | X | X | X |
| Lactic acid | X | X | X | X | X | X | X | X |
| Malonic acid | X | X | X | X | X | X | X | X |
| Decanoic Acid | X | X | X | X | X | X | X | X |
| Maleic acid | X | X | X | X | X | X | X | X |
| Glutaric acid | X | X | X | X | X | X | X | X |
| Citric acid | X | X | X | X | X | X | X | X |
| 3,3-dimethylacrylic acid | X | X | X | X | X | X | X | X |
| Gluconic acid | X | X | X | X | X | X | X | X |
| Adipic acid | X | X | X | X | X | X | X | X |
| 2-Ethylhexyl sulfate | X | X | X | X | X | X | X | X |
| 4-hydroxybenzenesulfonic | X | X | X | X | X | X | X | X |
| acid | | | | | | | | |
| Isovaleric acid | X | X | X | X | X | X | X | X |
| Hydrocinnamic acid | X | X | X | X | X | X | X | X |
| Phenylphosphoric acid | X | X | X | X | X | X | X | X |
| Biphenyl-3-carboxylic acid | X | X | X | X | X | X | X | X |

In some embodiments of any of the aspects, the ionic liquid is not CAGE (Choline And GEranate). In some embodiments of any of the aspects, the cation of the ionic liquid is not choline. In some embodiments of any of the aspects, the anion of the ionic liquid is not geranate or geranic acid. In some embodiments of any of the aspects comprising two or more ionic liquids, a first ionic liquid is not CAGE (Choline And GEranate). In some embodiments of any of the aspects comprising two or more ionic liquids, the cation of a first ionic liquid is not choline. In some embodiments of any of the aspects comprising two or more ionic liquids, the anion of a first ionic liquid is not geranate or geranic acid.

In some embodiments of any of the aspects, the anion is selected from the group consisting of geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid. In some embodiments of any of the aspects, the anion is selected from the group consisting of glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid.

In some embodiments of any of the aspects, the composition comprises a first ionic liquid and at least a second ionic liquid. Combinations of two, three, four, five, or more of any of the ionic liquids described herein are contemplated. By way of non-limiting example, the following table comprises exemplary pairwise combinations of ionic liquids that are contemplated herein.

| | | choline and | choline and | choline and | choline and | | choline and |
|---|---|---|---|---|---|---|---|
| | choline and | dimethylacrylic | isovaleric | phenylphosphoric | biphenyl-3- | choline and | phenylpropanoic |
| | geranic acid | acid | acid | acid | carboxylic acid | 4-phenolsulfonic | acid |
| | (CAGE) | (CADA) | (CAVA) | (CAPP) | (CABA) | acid (CASA) | (CAPA) |
| choline and | | x | x | x | x | x | X |
| geranic acid | | | | | | | |
| (CAGE) | | | | | | | |
| choline and | x | | x | x | x | x | x |
| dimethylacrylic | | | | | | | |
| acid (CADA) | | | | | | | |
| choline and | x | x | | x | x | x | x |
| isovaleric acid | | | | | | | |
| (CAVA) | | | | | | | |
| choline and | x | x | x | | x | x | x |
| phenylphosphoric | | | | | | | |
| acid (CAPP) | | | | | | | |
| choline and | x | x | x | x | | x | x |
| biphenyl-3- | | | | | | | |
| carboxylic acid | | | | | | | |
| (CABA) | | | | | | | |
| choline and 4- | x | x | x | x | x | | x |
| phenolsulfonic | | | | | | | |
| acid (CASA) | | | | | | | |

In some embodiments of any of the aspects wherein the composition comprises two or more ionic liquids, a first and second ionic liquid have the same cation, e.g., choline. In some embodiments of any of the aspects wherein the composition comprises two or more ionic liquids, a first and second ionic liquid have different anions. For example, a first ionic liquid and a second ionic liquid can each comprise a different anion selected from: geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid. In some embodiments of any of the aspects wherein the composition comprises two or more ionic liquids, the first ionic liquid has a geranic acid anion and the second ionic liquid has a phenylpropanoic acid anion.

In some embodiments of any of the aspects wherein the composition comprises two or more ionic liquids, the first ionic liquid is choline and geranic acid (CAGE). In some embodiments of any of the aspects wherein the composition comprises two or more ionic liquids, the second ionic liquid is choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).

In some embodiments of any of the aspects wherein the composition comprises two or more ionic liquids, the first and second ionic liquids are different ionic liquids selected from the group consisting of: choline and geranic acid (CAGE); choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA). In some embodiments of any of the aspects wherein the composition comprises two or more ionic liquids, the first ionic liquid is selected from the group consisting of: choline and geranic acid (CAGE); choline and dimethylacrylic acid (CADA); and choline and choline and biphenyl-3-carboxylic acid (CABA); and the second ionic liquid is selected from the group consisting of: isovaleric acid (CAVA); and choline and phenylpropanoic acid (CAPA). In some embodiments of any of the aspects wherein the composition comprises two or more ionic liquids, the first ionic liquid is choline and geranic acid (CAGE) and the second ionic liquid is choline and phenylpropanoic acid (CAPA).

In some embodiments of any of the aspects, the IL is at a concentration of at least 0.01% w/v. In some embodiments of any of the aspects, the IL is at a concentration of at least 0.05% w/v. In some embodiments of any of the aspects, the IL is at a concentration of at least 0.1% w/v. In some embodiments of any of the aspects, the IL is at a concentration of at least 0.2% w/v, at least 0.3% w/v, at least 0.4% w/v, at least 0.5% w/v, at least 1% w/v or greater. In some embodiments of any of the aspects, the IL is at a concentration of from about 0.01% w/v to about 1% w/v. In some embodiments of any of the aspects, the IL is at a concentration of from 0.01% w/v to 1% w/v. In some embodiments of any of the aspects, the IL is at a concentration of from about 0.05% w/v to about 0.5% w/v. In some embodiments of any of the aspects, the IL is at a concentration of from 0.05% w/v to 0.5% w/v.

In some embodiments of any of the aspects, the IL is at a concentration of at least 25% w/w. In some embodiments of any of the aspects, the IL is at a concentration of at least 25% w/w in water. In some embodiments of any of the aspects, the IL is at a concentration of at least 25% w/w in saline or a physiologically compatible buffer.

In some embodiments of any of the aspects, the IL is at a concentration of from about 5% w/w to about 75% w/w. In some embodiments of any of the aspects, the IL is at a concentration of from 5% w/w to 75% w/w. In some embodiments of any of the aspects, the IL is at a concentration of from about 5% w/w to about 75% w/w in water, saline or a physiologically compatible buffer. In some embodiments of any of the aspects, the IL is at a concentration of from 5% w/w to 75% w/w in water, saline or a physiologically compatible buffer.

In some embodiments of any of the aspects, the IL is at a concentration of at least about 0.1% w/w. In some embodiments of any of the aspects, the IL is at a concentration of at least 0.1% w/w. In some embodiments of any of the aspects, the IL is at a concentration of from about 10% w/w to about 70% w/w. In some embodiments of any of the aspects, the IL is at a concentration of from 10% w/w to 70% w/w. In some embodiments of any of the aspects, the IL is at a concentration of from about 30% w/w to about 50% w/w. In some embodiments of any of the aspects, the IL is at a concentration of from 30% w/w to 40% w/w. In some embodiments of any of the aspects, the IL is at a concentration of from about 30% w/w to about 50% w/w. In some embodiments of any of the aspects, the IL is at a concentration of from 30% w/w to 40% w/w.

In some embodiments of any of the aspects, the % w/w concentration of the IL is % w/w concentration in water, saline, or a physiologically compatible buffer.

In some embodiments of any of the aspects, the IL is 100% by w/w or w/v.

In some embodiments, the IL is an anhydrous salt, e.g., an ionic liquid not diluted or dissolved in water. In some embodiments, the IL is provided as an aqueous solution.

In some embodiments of any of the aspects, the IL is at a concentration of at least 25% w/w and has a ratio of cation:anion of at least 1:3. In some embodiments of any of the aspects, the IL is at a concentration of at least 25% w/w in water and has a ratio of cation:anion of at least 1:3. In some embodiments of any of the aspects, the IL is at a concentration of at least 25% w/w and has a ratio of cation:anion of 1:3 or 1:4. In some embodiments of any of the aspects, the IL is at a concentration of at least 25% w/w in water and has a ratio of cation:anion of 1:3 or 1:4. In some embodiments of any of the aspects, the IL is a gel, or a shear-thinning Newtonian gel.

In some embodiments of any of the aspects, the IL has a ratio of cation:anion of from about 10:1 to about 1:10. In some embodiments of any of the aspects, the IL has a ratio of cation:anion of from 10:1 to 1:10. In some embodiments of any of the aspects, the IL has a ratio of cation:anion of from about 5:1 to about 1:5. In some embodiments of any of the aspects, the IL has a ratio of cation:anion of from 5:1 to 1:5. In some embodiments of any of the aspects, the IL has a ratio of cation:anion of from about 2:1 to about 1:4. In some embodiments of any of the aspects, the IL has a ratio of cation:anion of from 2:1 to 1:4. In some embodiments of any of the aspects, the IL has a ratio of cation:anion of from about 2:1 to about 1:10. In some embodiments of any of the aspects, the IL has a ratio of cation:anion of from about 2:1 to about 1:1. In some embodiments of any of the aspects, the IL has a ratio of cation:anion of from 2:1 to 1:10. In some embodiments of any of the aspects, the IL has a ratio of cation:anion of from 2:1 to 1:1. In some embodiments of any of the aspects, the IL has a ratio of cation:anion such that there is a greater amount of anion, e.g., a ratio of less than 1:1. In some embodiments of any of the aspects, the IL has a ratio of cation:anion such that there is an excess of anion. In some embodiments of any of the aspects, the IL has a ratio of cation:anion of from about 1:1 to about 1:10. In some embodiments of any of the aspects, the IL has a ratio of cation:anion of from 1:1 to 1:10. In some embodiments of any of the aspects, the IL has a ratio of cation:anion of from about 1:1 to about 1:4. In some embodiments of any of the aspects, the IL has a ratio of cation:anion of from 1:1 to 1:4. In some embodiments of any of the aspects, the IL has a ratio of cation:anion of from about 1:1 to about 1:3. In some embodiments of any of the aspects, the IL has a ratio of cation:anion of from 1:1 to 1:3. In some embodiments of any of the aspects, the IL has a ratio of cation:anion of from about 1:1 to about 1:2. In some embodiments of any of the aspects, the IL has a ratio of cation:anion of from 1:1 to 1:2. In some embodiments of any of the aspects, the IL has a ratio of cation:anion of about 1:1, 1:2, 1:3, or 1:4. In some embodiments of any of the aspects, the IL has a ratio of cation:anion of 1:1, 1:2, 1:3, or 1:4. In some embodiments of any of the aspects, the IL has a ratio of cation:anion less than about of 1:1. In some embodiments of any of the aspects, the IL has a ratio of cation:anion less than 1:1. Without wishing to be constrained by theory, compositions with higher amounts of anion relative to cation display greater hydrophobicity.

In some embodiments of any of the aspects, the IL has a cation:anion ratio with an excess of cation.

In some embodiments of any of the aspects, e.g., when one or more nucleic acid molecules are provided in combination with the IL, the ratio of cation:anion is greater than 1:1, e.g., greater than 1:2, from about 1:2 to about 1:4, or from 1:2 to 1:4.

In some embodiments of any of the aspects, the IL is at a concentration of at least 20 mM. In some embodiments of any of the aspects, the IL is at a concentration of at least about 20 mM. In some embodiments of any of the aspects, the IL is at a concentration of at least 25 mM. In some embodiments of any of the aspects, the IL is at a concentration of at least about 25 mM. In some embodiments of any of the aspects, the IL is at a concentration of at least 50 mM. In some embodiments of any of the aspects, the IL is at a concentration of at least about 50 mM. In some embodiments of any of the aspects, the IL is at a concentration of at least 100 mM, 500 mM, 1 M, 2 M, 3 M or greater. In some embodiments of any of the aspects, the IL is at a concentration of at least about 100 mM, 500 mM, 1 M, 2 M, 3 M or greater.

In some embodiments of any of the aspects, the IL is at a concentration of from about 50 mM to about 4 M. In some embodiments of any of the aspects, the IL is at a concentration of from 50 mM to 4 M. In some embodiments of any of the aspects, the IL is at a concentration of from about 500 mM to about 4 M. In some embodiments of any of the aspects, the IL is at a concentration of from 500 mM to 4 M. In some embodiments of any of the aspects, the IL is at a concentration of from about 1 M to about 4 M. In some embodiments of any of the aspects, the IL is at a concentration of from 1 M to 4 M. In some embodiments of any of the aspects, the IL is at a concentration of from about 2 M to about 4 M. In some embodiments of any of the aspects, the IL is at a concentration of from 2 M to 4 M.

In some embodiments of any of the aspects, the IL concentration in the composition or formulation is about 0.1 mM to 20 mM. In some embodiments of any of the aspects, the IL concentration in the composition or formulation is about 0.5 mM to 20 mM, 0.5 mM to 18 mM, 0.5 mM to 16 mM, 0.5 mM to 14 mM, 0.5 mM to 12 mM, 0.5 mM to 10 mM, 0.5 mM to 8 mM, 1 mM to 20 mM, 1 mM to 18 mM, 1 mM to 16 mM, 1 mM to 14 mM, 1 mM to 12 mM, 1 mM to 10 mM, 1 mM to 8 mM, 2 mM to 20 mM, 2 mM to 18 mM, 2 mM to 16 mM, 2 mM to 14 mM, 2 mM to 12 mM, 2 mM to 10 mM, 2 mM to 8 mM, 4 mM to 20 mM, 4 mM to 18 mM, 4 mM to 16 mM, 4 mM to 14 mM, 4 mM to 12 mM, 4 mM to 10 mM, 4 mM to 8 mM, 6 mM to 20 mM, 6 mM to 18 mM, 6 mM to 14 mM, 6 mM to 12 mM, 6 mM to 10 mM, 6 mM to 8 mM, 8 mM to 20 mM, 8 mM to 18 mM, 8 mM to 16 mM, 8 mM to 14 mM, 8 mM to 12 mM, 8 mM to 10 mM, 10 mM to 20 mM, 10 mM to 18 mM, 10 mM to 16 mM, 10 mM to 14 mM, 10 mM to 12 mM, 12 mM to 20 mM, 12 mM to 18 mM, 12 mM to 16 mM, 12 mM to 14 mM, 14 mM to 20 mM, 14 mM to 18 mM, 14 mM to 16 mM, 16 mM to 20 mM, 16 mM to 18 mM, or 18 mM to 20 mM. In some embodiments of any of the aspects, the IL concentration in the composition or formulation is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

It is specifically contemplated that a composition or combination described herein can comprise one, two, three, or more of any of the types of components described herein. For example, a composition can comprise a mixture, solution, combination, or emulsion of two or more different ionic liquids (e.g., different ionic liquids described herein), and/or a mixture, solution, combination, or emulsion of two or more different non-ionic surfactants, and/or a mixture, solution, combination, or emulsion of two or more different active compounds.

In some embodiments of any of the aspects, the one or more ILs can be in combination with at least one compound. As used herein, “in combination with” refers to two or more substances being present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the active compound(s) can be comprised by a superstructure, e.g., nanoparticles, liposomes, vectors, cells, scaffolds, or the like, said superstructure is which in solution, mixture, admixture, suspension, etc., with the IL.

As used herein, an “active compound” or “active agent” is any agent which will exert an effect on a target cell or organism. The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally-occurring non-proteinaceous entities. Agents can be known to have a desired activity and/or property, or can be selected from a library of diverse compounds. Non-limiting examples of active compounds contemplated for use in the methods described herein include small molecules, polypeptides, nucleic acids, chemotherapies/chemotherapeutic compounds, antibodies, antibody reagents, vaccines, a GLP-1 polypeptide or mimetic/analog thereof, insulin, acarbose, or ruxolitinib.

A nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other nucleic acid molecule that one wishes to deliver to a cell. The nucleic acid molecule can be RNA, DNA, or synthetic or modified versions thereof. In some embodiments of any of the aspects, the nucleic acid is an inhibitory nucleic acid, e.g., a siRNA.

In one aspect of any of the embodiments, described herein is a method of delivering a nucleic acid molecule to a cell, the method comprising contacting the cell with the nucleic acid molecule in combination with one or more ILs as described herein. In some embodiments of any of the aspects, the cell is a cell in a subject and the contacting step comprises administering the nucleic acid molecule in combination with the one or more ILs to the subject. In some embodiments of any of the aspects, the cell is in vitro, in vivo, or ex vivo. In some embodiments of any of the aspects, the cell is eukaryotic. In some embodiments of any of the aspects, the cell is mammalian. In some embodiments of any of the aspects, the cell is an epithelial cell, e.g., an intestinal epithelial cell. In some embodiments of any of the aspects the cell is an epidermal cell.

In some embodiments of any of the aspects, wherein the active compound comprises a nucleic acid, the anion has a LogP of less than 1.0 and is a) a carboxylic acid which is not a fatty acid; or b) a carboxylic acid comprising an aliphatic chain of no more than 4 carbons; or c) an aromatic anion. In some embodiments of any of the aspects, wherein the active compound comprises a nucleic acid, the anion has a LogP of less than 1.0 and is an aromatic anion. In some embodiments of any of the aspects, wherein the active compound comprises a nucleic acid, the anion is an aromatic anion.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments of any of the aspects, the active compound can be a therapeutic compound or drug, e.g., an agent or compound which is therapeutically effective for the treatment of at least one condition in a subject. Therapeutic compounds are known in the art for a variety of conditions, see, e.g., the database available on the world wide web at drugs.com or the catalog of FDA-approved compounds available on the world wide web at catalog.data.gov/dataset/drugsfda-database; each of which is incorporated by reference herein in its entirety.

By way of non-limiting example, exemplary antibodies and/or antibody reagents suitable for use as active compounds/therapeutic compounds herein include: abciximab; adalimumab; adlimumab-atto; ado-trastuzumab; ado-trastuzumab emtansine; alemtuzumab; alirocumab; atezolizumab; avelumab; basiliximab; belimumab; bevacizumab; bezlotoxumab; blinatumomab; brentuximab; brentuximab vedotin; brodalumab; canakinumab; capromab; capromab pendetide; certolizumab; certolizumab pegol; cetuximab; daclizumab; daratumumab; denosumab; dinutuximab; dupilumab; durvalumab; eculizumab; elotuzumab; evolocumab; etanercept; etanercept-szzs; golimumab; ibritumomab; ibritumomab tiuxetan; idarucizumab; infliximab; infliximab-abda; infliximab-dyyb; ipilimumab; ixekizumab; mepolizumab; natalizumab; necitumumab; nivolumab; obiltoxaximab; obinutuzumab; ocrelizumab; ofatumumab; olaratumab; omalizumab; palivizumab; panitumumab; pembrolizumab; pertuzumab; ramucriumab; ranibizumab; raxibacumab; reslizumab; rituximab; secukinumab; siltuximab; tocilizumab; trastuzumab; ustekinumab; vedolizumab; sarilumab; guselkumab; inotuzumab ozogamicin; inotuzumab; adalimumab-adbm, gemtuzumab ozogamicin; gemtuzumab; bevacizumab-awwb; benralizumab; emicizumab; emicizumab-kxwh; trastuzumab-dkst; infliximab-qbtx; ibalizumab; ibalizumab-uiyk; tildrakizumab; tildrakizumab-asmn; burosumab; burosumab-twza; erenumab; erenumab-aooe; tositumomab; mogamulizumab; moxetumomab; moxetumomab pasudotox; cemiplimab; polatuzumab; catumaxomab; polatuzumab vedotin; and combinations thereof, including bispecific antibodies made by combining portions of the foregoing.

By way of non-limiting example, exemplary inhibitory nucleic acids suitable for use as active compounds/therapeutic compounds herein include: patisiran; and combinations thereof, including bispecific antibodies made by combining portions of the foregoing.

As used herein the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule.

In some embodiments of any of the aspects, the active compound is a polypeptide. In some embodiments of any of the aspects, the active compound is an antibody or antibody reagent. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

In some embodiments of any of the aspects, wherein the active compound comprises a polypeptide (e.g., an antibody or antibody reagent), the anion has a LogP of less than 1.0 and is a) a carboxylic acid which is not a fatty acid; or b) a carboxylic acid comprising an aliphatic chain of no more than 4 carbons; or c) an aromatic anion. In some embodiments of any of the aspects, wherein the active compound comprises a polypeptide (e.g., an antibody or antibody reagent), the anion has a LogP of less than 1.0 and is a) a carboxylic acid which is not a fatty acid; or b) a carboxylic acid comprising an aliphatic chain of no more than 4 carbons. In some embodiments of any of the aspects, wherein the active compound comprises a polypeptide (e.g., an antibody or antibody reagent), the anion has a LogP of less than 1.0 and is a carboxylic acid comprising an aliphatic chain of no more than 4 carbons. In some embodiments of any of the aspects, wherein the active compound comprises a polypeptide (e.g., an antibody or antibody reagent), is a) a carboxylic acid which is not a fatty acid; or b) a carboxylic acid comprising an aliphatic chain of no more than 4 carbons. In some embodiments of any of the aspects, wherein the active compound comprises a polypeptide (e.g., an antibody or antibody reagent), the anion has a LogP of less than 1.0.

In some embodiments of any of the aspects, the active compound has a molecular weight of greater than about 450. In some embodiments of any of the aspects, the active compound has a molecular weight of greater than about 500. In some embodiments of any of the aspects, the active compound has a molecular weight of greater than 450, e.g., greater than 450, greater than 500, greater than 550, greater than 600, greater than 1000 or more. In some embodiments of any of the aspects, the active compound is polar.

In some embodiments of any of the aspects wherein the active agent is an inhibitory nucleic acid, the composition comprises two or more ionic liquids and the first ionic liquid is choline and geranic acid (CAGE). In some embodiments of any of the aspects wherein the active agent is an inhibitory nucleic acid, the composition comprises two or more ionic liquids, and the second ionic liquid is choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).

In some embodiments of any of the aspects wherein the active agent is an inhibitory nucleic acid, the composition comprises two or more ionic liquids, the first and second ionic liquids are different ionic liquids selected from the group consisting of: choline and geranic acid (CAGE); choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA). In some embodiments of any of the aspects wherein the active agent is an inhibitory nucleic acid, the composition comprises two or more ionic liquids, the first ionic liquid is selected from the group consisting of: choline and geranic acid (CAGE); choline and dimethylacrylic acid (CADA); and choline and choline and biphenyl-3-carboxylic acid (CABA); and the second ionic liquid is selected from the group consisting of: isovaleric acid (CAVA); and choline and phenylpropanoic acid (CAPA). In some embodiments of any of the aspects wherein the active agent is an inhibitory nucleic acid, the composition comprises two or more ionic liquids, the first ionic liquid is choline and geranic acid (CAGE) and the second ionic liquid is choline and phenylpropanoic acid (CAPA). In some embodiments of any of the aspects, the composition is administered topically, or is formulated for topical administration.

In some embodiments, the inhibitory nucleic acid is a NFKBIZ inhibitory nucleic acid, e.g., it binds to a NFKBIZ mRNA and inhibits the expression of NFKBIZ. As used herein, “NFKBIZ” or “NFKB inhibitor zeta” refers to an inhibitor of nuclear factor κB (IκB) protein IκBζ, that plays a key role in the regulation of NF-κB complexes. It is a direct transcription activator of TNF-α-, IL-17A-, and IL-36-inducible psoriasis-related gene products that are involved in inflammatory signaling, neutrophil chemotaxis, and leukocyte activation. Accordingly, provided herein are methods of treating psoriasis, e.g., by administering a composition described herein comprising an active agent that is an inhibitor of NFKBIZ, e.g., a NFKBIZ inhibitory nucleic acid. Sequences of NFKBIZ from a number of species are known in the art, e.g., human NFKBIZ sequences are available in the NCBI database under the 64332 Gene ID (e.g., mRNAs NM_001005474.3 (SEQ ID NO: 37) and NM_031419.4 (SEQ ID NO: 38)). One of skill in the art can readily design an NFKBIZ inhibitory nucleic acid, e.g., using an automated tool as described above herein. NKFBIZ inhibitory nucleic acids are also commercially available, e.g., catalog no. J-040680-06-0050 from Dharmacon (Lafayette, CO).

In some embodiments, the inhibitory nucleic acid is a TNF-alpha inhibitory nucleic acid, e.g., it binds to a TNF-alpha mRNA and inhibits the expression of TNF-alpha. As used herein, “tumor necrosis factor alpha” or “TNF-alpha” refers to a pro-inflammatory cytokine implicated in autoimmune disease, psoriasis, and other conditions. Accordingly, provided herein are methods of treating inflammatory conditions (e.g., psoriasis) and/or reducing or inhibition inflammation, e.g., by administering a composition described herein comprising an active agent that is an inhibitor of TNF-alpha, e.g., a TNF-alpha inhibitory nucleic acid. Sequences of TNF-alpha from a number of species are known in the art, e.g., human TNF-alpha sequences are available in the NCBI database under the 7124 Gene ID (e.g., mRNA NM_000594.4 (SEQ ID NO: 39)). One of skill in the art can readily design a TNF-alpha inhibitory nucleic acid, e.g., using an automated tool as described above herein. TNF-alpha inhibitory nucleic acids are also commercially available, e.g., catalog nos. J-010546-09-0002, J-010546-10-0002, J-010546-11-0002, and J-010546-12-0002, from Dharmacon (Lafayette, CO).

In some embodiments, the inhibitory nucleic acid is an IL-17 inhibitory nucleic acid, e.g., it binds to an IL-17 mRNA and inhibits the expression of IL-17. As used herein, “interleukin 17” or “IL-17” refers to a pro-inflammatory cytokine produced by activating T cells implicated in autoimmune disease, psoriasis, rheumatoid arthritis, multiple sclerosis, and other conditions. Accordingly, provided herein are methods of treating inflammatory conditions (e.g., psoriasis) and/or reducing or inhibition inflammation, e.g., by administering a composition described herein comprising an active agent that is an inhibitor of IL-17, e.g., an IL-17 inhibitory nucleic acid. Sequences of IL-17 from a number of species are known in the art, e.g., human IL-17 sequences are available in the NCBI database under the 3605 Gene ID (e.g., mRNA NM_002190.3 (SEQ ID NO: 40)). One of skill in the art can readily design an IL-17 inhibitory nucleic acid, e.g., using an automated tool as described above herein. IL-17 inhibitory nucleic acids are also commercially available, e.g., catalog no. J-007937-05-0002, J-007937-06-0002, J-007937-07-0002, and J-007937-08-0002 from Dharmacon (Lafayette, CO).

In one aspect of any of the embodiments, provided herein is a method of treating an inflammatory condition and/or a method of reducing inflammation in a subject in need thereof, the method comprising administering a composition described herein, comprising at least one IL and at least one anti-inflammatory agent to the subject. In some embodiments of any of the aspects, the anti-inflammatory agent is an inhibitory nucleic acid that targets one or more pro-inflammatory gene products, e.g., IL-17, TNF-alpha, and/or NFKBIZ.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, the inflammatory condition is an inflammatory condition of the skin. In some embodiments of the aspects, the inflammatory condition is an autoimmune disease.

Non-limiting examples of inflammatory conditions of the skin can include psoriasis, such as Sweet's syndrome, pyoderma gangrenosum, subcomeal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, atherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-lassociated myelopathy/tropical spactic paraparesis; Devic's disease; human immunodeficiency virus encephalopathy; human immunodeficiency virus vacuolar myelopathy; peripheral neuropathies; Guillain-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g., viral, bacterial, fungal, parasite or prion infections. In some embodiments, an inflammatory condition is associated with an allergic response. In some embodiments, an inflammatory condition is associated with a pollutant (e.g., asbestosis, silicosis, or berylliosis).

In some embodiments, the inflammatory condition can be a local condition, e.g., a rash or allergic reaction. In some embodiments, the inflammation is associated with a wound.

Anti-inflammatory agents are known in the art and can include, by way of non-limiting example, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and inhibitors of pro-inflammatory gene products (e.g., inhibitory nucleic acids as described above herein). Pro-inflammatory genes are known in the art and include, by way of non-limiting example, NKFBIZ, TNF-alpha, IL-17, IL-36 (IL-37alpha, IL-36beta, and IL-36gamma), IL-22, IL-17C, CXCL8, CCL20, IL23A, DEFB4, and LCN2.

As used herein, “composition” refers to any IL, combination of ILs, or combination of one or more ILs and one or more active agents described herein, unless further specified.

In some embodiments of any of the aspects, a composition or combination as described herein, comprising at least one IL and optionally an active compound can be formulated as an oral, subcutaneous, transdermal, intratumoral, intravenous, intradermal, or parenteral formulation. In some embodiments of any of the aspects, the composition or combination as described herein can be formulated for delivery to a mucus membrane, e.g., to a nasal, oral, or vaginal membrane. In some embodiments of any of the aspects, an oral formulation can be a degradable capsule comprising the composition comprising the at least one IL and optionally, an active compound.

In some embodiments of any of the aspects, described herein is a composition comprising at least one IL as described herein and at least one active compound. In some embodiments of any of the aspects, described herein is a composition consisting essentially of at least one IL as described herein and at least one active compound. In some embodiments of any of the aspects, described herein is a composition consisting of at least one IL as described herein and at least one active compound. In some embodiments of any of the aspects, the composition comprising at least one IL as described herein and at least one active compound is administered as a monotherapy, e.g., another treatment for the condition is not administered to the subject.

In one aspect of any of the embodiments, described herein is a pharmaceutical composition comprising at least one active compound in combination with at least one IL as described herein. In some embodiments, the pharmaceutical composition comprises the at least one IL and the one or more active compounds as described herein. In some embodiments, the pharmaceutical composition consists essentially of the at least one IL and the one or more active compounds as described herein. In some embodiments, the pharmaceutical composition consists of the at least one IL and the one or more active compounds as described herein. In some embodiments, the pharmaceutical composition consists essentially of an aqueous solution of the at least one IL and the one or more active compounds as described herein. In some embodiments, the pharmaceutical composition consists of an aqueous solution of the at least one IL and the one or more active compounds as described herein.

The compositions, formulations, and combinations described herein can comprise at least one IL as described herein, e.g., one IL, two ILs, three ILs, or more. In some embodiments of any of the aspects, a composition, formulation, or combination as described herein can comprise at least one IL as described herein and CAGE (Choline And GEranate).

In some embodiments of any of the aspects, the at least one active compound and the at least one ionic liquid are further in combination with at least one non-ionic surfactant. As used herein, “non-ionic surfactant” refers to a surfactant which lacks a net ionic charge and does not dissociate to an appreciable extent in aqueous media. The properties of non-ionic surfactants are largely dependent upon the proportions of the hydrophilic and hydrophobic groups in the molecule. Hydrophilic groups include the oxyethylene group (—OCH2 CH2-) and the hydroxy group. By varying the number of these groups in a hydrophobic molecule, such as a fatty acid, substances are obtained which range from strongly hydrophobic and water insoluble compounds, such as glyceryl monostearate, to strongly hydrophilic and water-soluble compounds, such as the macrogols. Between these two extremes types include those in which the proportions of the hydrophilic and hydrophobic groups are more evenly balanced, such as the macrogol esters and ethers and sorbitan derivatives. Suitable non-ionic surfactants may be found in Martindale, The Extra Pharmacopoeia, 28th Edition, 1982, The Pharmaceutical Press, London, Great Britain, pp. 370 to 379. Non-limiting examples of non-ionic surfactants include polysorbates, a Tween™, block copolymers of ethylene oxide and propylene oxide, glycol and glyceryl esters of fatty acids and their derivatives, polyoxyethylene esters of fatty acids (macrogol esters), polyoxyethylene ethers of fatty acids and their derivatives (macrogol ethers), polyvinyl alcohols, and sorbitan esters, sorbitan monoesters, ethers formed from fatty alcohols and polyethylene glycol, polyoxyethylene-polypropylene glycol, alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, lauryl glucoside, maltosides, monolaurin, mycosubtilin, Nonidet P-40, nonoxynol-9, nonoxynols, NP-40, octaethylene glycol monododecyl ether, N-Octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, sorbitan, sorbitan monolaurate, sorbitan monostearate, sorbitan tristearate, stearyl alcohol, surfactin, Triton X-100, and the like. In some embodiments of any of the aspects, the at least one non-ionic surfactant has a neutral hydrophilic head group.

As used herein, “polysorbate” refers to a surfactant derived from ethoxylated sorbitan (a derivative of sorbitol) esterified with fatty acids. Common brand names for polysorbates include Scattics™, Alkest™, Canarcel™, and Tween™. Exemplary polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

In some embodiments of any of the aspects, the at least one non-ionic surfactant (e.g., at least one polysorbate) is present at a concentration of about 0.10% to about 50% w/v. In some embodiments of any of the aspects, the at least one non-ionic surfactant (e.g., at least one polysorbate) is present at a concentration of 0.1% to 50% w/v. In some embodiments of any of the aspects, the at least one non-ionic surfactant (e.g., at least one polysorbate) is present at a concentration of about 1% to about 5% w/v. In some embodiments of any of the aspects, the at least one non-ionic surfactant (e.g., at least one polysorbate) is present at a concentration of 1% to 5% w/v. In some embodiments of any of the aspects, the at least one non-ionic surfactant (e.g., at least one polysorbate) is present at a concentration of about 3% to about 10% w/v. In some embodiments of any of the aspects, the at least one non-ionic surfactant (e.g., at least one polysorbate) is present at a concentration of 3% to 10% w/v. In some embodiments of any of the aspects, the at least one non-ionic surfactant (e.g., at least one polysorbate) is present at a concentration of less than about 5% w/v. In some embodiments of any of the aspects, the at least one non-ionic surfactant (e.g., at least one polysorbate) is present at a concentration of less than 5% w/v.

In some embodiments of any of the aspects, the combination of the at least one active compound and at least one IL as described herein is provided in one or more nanoparticles. In some embodiments of any of the aspects, the combination of the at least one active compound and at least one IL as described herein comprises nanoparticles comprising the active compound, the nanoparticles in solution or suspension in a composition comprising at least one IL as described herein.

In some embodiments of any of the aspects, a composition as described herein, e.g., a composition comprising at least one IL and an active compound, can further comprise a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

The term “carrier” in the context of a pharmaceutical carrier refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active compound. The term “pharmaceutically acceptable carrier” excludes tissue culture medium.

In some embodiments of any of the aspects, a composition as described herein, e.g., a composition comprising at least one IL as described herein and an active compound, can be formulated as an oral, subcutaneous, intravenous, intradermal, or parenteral formulation. In some embodiments of any of the aspects, an oral formulation can be a degradable capsule comprising the composition described herein, e.g., a composition comprising at least one IL as described herein and an active compound.

In some embodiments of any of the aspects described herein, the biological activity of the active compound is improved or stabilized as compared to the activity in the absence of the at least one IL. In some embodiments of any of the aspects described herein, the IL greatly enhances permeation of the active compound across the skin compared to a control where the at least one IL is absent.

In one aspect of any of the embodiments, described herein is a method of administering at least active compound to a subject using a catheter wherein the catheter is coated with at least one IL as described herein. In one aspect of any of the embodiments, described herein is a method of collecting a body fluid by placing the catheter into the body wherein the catheter is coated with at least one IL as described herein.

In one aspect of any of the embodiments, the composition or combination described herein is for a method of administering or delivering at least one active compound, e.g., for the treatment of a disease. In one aspect of any of the embodiments, described herein is a method of administering at least one active compound, the method comprising administering the active compound in combination with at least one IL as described herein. In one aspect of any of the embodiments, described herein is a method of treating a disease by administering at least one active compound, the method comprising administering the active compound in combination with at least one IL as described herein.

The disease treated by the methods described herein can be, e.g., cancer (breast cancer, leukemia, lymphoma, B-cell chronic lymphocytic leukemia, glioblastoma, carcinoma, urothelial carcinoma, lung cancer, colorectal cancer, lymphoblastic leukemia, lymphocytic leukemia, sarcoma, melanoma, prostate cancer, myeloma, multiple myeloma, Non-Hodgkin's lymphoma), neuroblastoma, diabetes, an infection, inflammation, inflammatory diseases (e.g., rheumatoid arthritis, juvenile idiopathic arthritis, psoriatic arthritis, ankylosing spondylitis, crohn's disease, ulcerative colitis, plaque psoriasis), autoimmune diseases, atopic dermatitis, gastrointestinal inflammation, inflammatory bowel disease (IBD), cholesterolemia, coronary artery disease, asthma, transplant/organ rejection, systemic lupus erythematosus, multiple sclerosis, osteoporosis, and the like.

In some embodiments, the methods described herein relate to treating a subject having or diagnosed as having a condition with a composition as described herein, e.g., a comprising at least one IL and an active compound. Subjects having a condition, e.g., diabetes, can be identified by a physician using current methods of diagnosing diabetes. Symptoms and/or complications of diabetes which characterize these conditions and aid in diagnosis are well known in the art and include but are not limited to, weight loss, slow healing, polyuria, polydipsia, polyphagiam headaches, itchy skin, and fatigue. Tests that may aid in a diagnosis of, e.g. diabetes include, but are not limited to, blood tests (e.g., for fasting glucose levels). A family history of diabetes, or exposure to risk factors for diabetes (e.g. overweight) can also aid in determining if a subject is likely to have diabetes or in making a diagnosis of diabetes.

The compositions and methods described herein can be administered to a subject having or diagnosed as having a condition described herein. In some embodiments, the methods described herein comprise administering an effective amount of compositions described herein, e.g. a composition comprising at least one IL as described herein and an active compound, to a subject in order to alleviate a symptom of a condition described herein. As used herein, “alleviating a symptom” is ameliorating any marker or symptom associated with a condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local or systemic.

In some embodiments of any of the aspects, the administration is transdermal. In some embodiments of any of the aspects, the administration is transdermal, to a mucus membrane (e.g., to a nasal, oral, or vaginal membrane), oral, subcutaneous, intradermal, parenteral, intratumoral, or intravenous.

Oral administration can comprise providing tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Oral formulations can comprise discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of CAGE and the at least one active compound, and may be prepared by methods of pharmacy well known to those skilled in the art. See generally, Remington: The Science and Practice of Pharmacy, 21st Ed., Lippincott, Williams, and Wilkins, Philadelphia PA. (2005).

In one aspect of any of the embodiments, described herein is a method of delivery of at least one active compound by subcutaneous, intradermal or intravenous administration, the method comprising administering the active compound in combination with at least one IL as described herein. In some embodiments of any of the aspects, subcutaneous, intradermal or intravenous administration comprises administration via injection, catheter, port, or the like.

In one aspect of any of the embodiments, described herein is a method of parenteral delivery of at least one active compound, the method comprising parenterally administering the active compound in combination with at least one IL as described herein. In some embodiments, the parenteral administration comprises delivery to a tumor, e.g., a cancer tumor. In some embodiments of any of the aspects, the composition or combination described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition comprising at least one IL (e.g., CAGE) in combination with at least one active compound as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of an ingredient in a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. While as noted above herein, the compositions comprising the at least one IL in combination with at least one active compound can obviate certain reasons for using a controlled-release formulation, it is contemplated herein that the methods and compositions can be utilized in controlled-release formulations in some embodiments. For example, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition comprising the at least one IL in combination with at least one active compound can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS© (Alza Corporation, Mountain View, Calif. USA)), or a combination thereof to provide the desired release profile in varying proportions.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for blood glucose, among others. The dosage can be determined by a physician and adjusted, as necessary, to suit observed effects of the treatment.

As used herein, “diabetes” refers to diabetes mellitus, a metabolic disease characterized by a deficiency or absence of insulin secretion by the pancreas. As used throughout, “diabetes” includes Type 1, Type 2, Type 3, and Type 4 diabetes mellitus unless otherwise specified herein. The onset of diabetes is typically due to a combination of hereditary and environmental causes, resulting in abnormally high blood sugar levels (hyperglycemia). The two most common forms of diabetes are due to either a diminished production of insulin (in type 1), or diminished response by the body to insulin (in type 2 and gestational). Both lead to hyperglycemia, which largely causes the acute signs of diabetes: excessive urine production, resulting compensatory thirst and increased fluid intake, blurred vision, unexplained weight loss, lethargy, and changes in energy metabolism. Diabetes can cause many complications. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation. In some embodiments, the diabetes can be Type 2 diabetes. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. In some embodiments, a subject can be pre-diabetic, which can be characterized, for example, as having elevated fasting blood sugar or elevated post-prandial blood sugar.

Glucagon-Like Peptide-1 (GLP-1), is known to reduce food intake and hunger feelings in humans and is an incretin derived from the transcription product of the proglucagon gene that contributes to glucose homeostasis. GLP-1 mimetics are currently being used in the treatment of Type 2 diabetes. Recent clinical trials have shown that these treatments not only improve glucose homeostasis but also succeed in inducing weight loss. As used herein. “GLP-1 polypeptide” refers to the various pre- and pro-peptides and cleavage products of GLP-1, e.g., for human: GLP-1(1-37) (SEQ ID NO: 2), GLP-1 (7-36) (SEQ ID NO: 3), and GLP-1 (7-37) (SEQ ID NO: 4). In some embodiments, a GLP-1 polypeptide can be GLP-1 (7-36) and/or GLP-1 (7-37) or the correlating polypeptides from a species other than human. Sequences for GLP-1 polypeptides are known in the art for a number of species, e.g. human GLP-1 (NCBI Gene ID: 2641) polypeptides (e.g., NCBI Ref Seq: NP_002045.1; SEQ ID NO: 1) and SEQ ID NOs: 2-4. In some embodiments, a pre or pro-peptide of GLP-1 can be used in the methods or compositions described herein, e.g., a glucagon preproprotein (e.g., SEQ ID NO: 1). Naturally-occurring alleles or variants of any of the polypeptides described herein are also specifically contemplated for use in the methods and compositions described herein.

| SEQ ID NO: 1 | |
|---|---|
| 1 | mksiyfvagl fvmlvqgswq rslqdteeks rsfsasqadp lsdpdqmned krhsqgtfts |
| 61 | dyskyldsrr aqdfvqwlmn tkrnrnniak rhdeferhae gtftsdvssy legqaakefi |
| 121 | awlvkgrgrr dfpeevaive elgrrhadgs fsdemntild nlaardfinw liqtkitdrk |

SEQ ID NO: 2
hdeferhae gtftsdvssy legqaakefi awlvkgrg
SEQ ID NO: 3
hae gtftsdvssy legqaakefi awlvkgr
SEQ ID NO: 4
hae gtftsdvssy legqaakefi awlvkgrg

Various GLP-1 mimetics are known in the art and used in the treatment of diabetes. GLP-1 mimetics (or analogues) can include exendin-4 (a Heloderma lizard polypeptide with homology to human GLP-1) and derivatives thereof, GLP-1 analogs modified to be DPP-IV resistant, or human GLP-1 polypeptides conjugated to various further agents, e.g., to extend the half-life. GLP-1 mimetics/analogues can include, e.g., exenatide, lixisenatide, dulaglutide, semaglutide, albiglutide, LY2189265, liraglutide, and taspoglutide. Examples of such molecules and further discussion of their manufacture and activity can be found in the art, e.g., Gupta. Indian J. Endocrinol Metab 17:413-421 (2013); Garber. Diabetes Treatments 41:S279-S284 (2018); US Patent Publication US2009/0181912; and International Patent Publication WO2011/080103, each of which is incorporated by reference herein in its entirety.

In some embodiments of any of the aspects, the active compound can be a chemotherapeutic agent or agent effective for the treatment of cancer. As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the composition as described herein, e.g., a composition comprising at least one IL as described herein in combination with at least one active compound, is administered as a monotherapy, e.g., another treatment for the condition is not administered to the subject.

In some embodiments of any of the aspects, the methods described herein can further comprise administering a second agent and/or treatment to the subject, e.g. as part of a combinatorial therapy, either in the composition described herein, e.g., a composition comprising at least one IL as described herein in combination with at least one active compound, or as a separate formulation. For example, non-limiting examples of a second agent and/or treatment for treatment of cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition described herein, e.g., a composition comprising at least one IL as described herein in combination with at least one active compound, can be administered to a patient once. In certain embodiments, an effective dose a composition described herein, e.g., a composition comprising at least one IL as described herein in combination with at least one active compound, can be administered to a patient repeatedly. For systemic administration, subjects can be administered a therapeutic amount of a composition described herein, e.g., a composition comprising at least one IL as described herein in combination with at least one active compound, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. In some embodiments of any of the aspects, the at least one active compound is present in the combination at a dose of from about 1.0-40.0 mg/kg. In some embodiments of any of the aspects, the at least one active compound is present in the combination at a dose of from 1.0-40.0 mg/kg. In some embodiments of any of the aspects, the at least one active compound is present in the combination at a dose of from about 1.0-20.0 mg/kg. In some embodiments of any of the aspects, the at least one active compound is present in the combination at a dose of from 1.0-20.0 mg/kg.

In some embodiments, the active compound is insulin and the concentration or dosage of insulin can be from about 1 U/kg to about 20 U/kg. In some embodiments, the active compound is insulin and the concentration or dosage of insulin can be from 1 U/kg to 20 U/kg. In some embodiments, the active compound is insulin and the concentration or dosage of insulin can be less than 20 U/kg. In some embodiments, the active compound is insulin and the concentration or dosage of insulin can be from about 2 U/kg to about 10 U/kg. In some embodiments, the active compound is insulin and the concentration or dosage of insulin can be from 2 U/kg to 10 U/kg. In some embodiments, the active compound is insulin and the concentration or dosage of insulin can be from about 2 U/kg to about 5 U/kg. In some embodiments, the active compound is insulin and the concentration or dosage of insulin can be from 2 U/kg to 5 U/kg. In some embodiments, the active compound is insulin and the concentration or dosage of insulin can be from about 5 U/kg to about 10 U/kg. In some embodiments, the active compound is insulin and the concentration or dosage of insulin can be from 5 U/kg to 10 U/kg. In some embodiments, the active compound is insulin and the concentration or dosage of insulin can be 2 U/kg, 5 U/kg, or 10 U/kg.

In one aspect of any of the embodiments, described herein is a method of treating a disease in a subject in need thereof by administering to the subject an active compound in combination with the at least one IL as described herein by into the affected tissue by injection. In some embodiments, the affected tissue is tissue comprising diseased cells. In some embodiments, the affected tissue is tissue displaying symptoms of the disease. Non-limiting examples of suitable affected tissues include tumor tissue, fat tissue, adipose tissue, or the like. In some embodiments of any of the aspects, suitable affected tissues include tumor tissue, fat tissue, adipose tissue, or the like. In some embodiments of any of the aspects, the disease is a disease arising from tissue growth, e.g., unwanted, aberrant, or pathological tissue growth. A disease arising from tissue growth can be any disease caused by or characterized by, a rate of tissue growth, location of tissue growth, or pattern/structure of tissue growth which differs from what is normal for that tissue type in a healthy subject. Non-limiting examples of such diseases are tumors, cancer, fat/obesity, and/or hyperplasia. In some embodiments of any of the aspects, such diseases are tumors, cancer, fat/obesity, and/or hyperplasia.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active compound. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition described herein, e.g., a composition comprising at least one IL in combination with at least one active compound, can be administered over a period of time, such as over a 5 minute, 10 minute, 15 minute, 20 minute, or 25 minute period.

The dosage ranges for the administration of the compositions described herein, according to the methods described herein depend upon, for example, the form of the active compound, its potency, and the extent to which symptoms, markers, or indicators of a condition described herein are desired to be reduced, for example the percentage reduction desired for symptoms or markers. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition described in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of diabetes or cancer. When using an experimental animal model, efficacy of treatment is evidenced when a statistically significant change in a marker is observed.

In vitro and animal model assays are provided herein which allow the assessment of a given dose of a composition described herein, e.g., a composition comprising at least one IL in combination with at least one active compound.

In some embodiments of any of the aspects, the subject administered a composition comprising at least one IL as described herein, e.g., in combination with an active compound is a subject having, diagnosed as having, or in need of treatment for obesity, excess weight, or prevention of weight gain. In some embodiments, the subject is overweight. The methods described herein comprises methods of treating obesity, reducing weight gain, preventing weight gain, promoting weight loss, and the like. Such methods can, e.g., promote metabolic health, be pursued for aesthetic reasons, and/or prepare patients for surgical interventions which are counter indicated for those with high BMIs or weights. In some embodiments, weight loss can be medically necessary and/or medically indicated, e.g. when the subject is overweight and/or obese. In some embodiments, weight loss can be for cosmetic purposes, e.g. when the subject desires to lose weight whether or not weight loss is medically necessary and/or medically indicated.

The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Some common conditions related to high BMI and obesity include cardiovascular disease, high blood pressure (i.e., hypertension), osteoarthritis, cancer, and diabetes. Although BMI correlates with body fat, the relation between BMI and actual body fat differs with age and gender. For example, women are more likely to have a higher percent of body fat than men for the same BMI. Furthermore, the BMI threshold that separates normal, overweight, and obese can vary, e.g. with age, gender, ethnicity, fitness, and body type, amongst other factors. In some embodiments, a subject with obesity can be a subject with a body mass index of at least about 25 kg/m² prior to administration of a treatment as described herein. In some embodiments, a subject with obesity can be a subject with a body mass index of at least about 30 kg/m² prior to administration of a treatment as described herein.

In some embodiments of any of the aspects, the subject administered a composition comprising at least one IL as described herein, e.g., in combination with at least one active compound is a subject having, diagnosed as having, or in need of treatment for a metabolic disorder or metabolic syndrome. The term “metabolic disorder” refers to any disorder associated with or aggravated by impaired or altered glucose regulation or glycemic control, such as, for example, insulin resistance. Such disorders include, but are not limited to obesity; excess adipose tissue; diabetes; fatty liver disease; non-alcoholic fatty liver disease; metabolic syndrome; dyslipidemia; hypertension; hyperglycemia; and cardiovascular disease. “Metabolic syndrome”, which is distinct from metabolic disorder, refers to a combination of medical disorders that, when occurring together, increase the risk of developing cardiovascular disease and diabetes. A number of definitions of metabolic syndrome have been established, e.g., by the American Heart Association and the International Diabetes Foundation. As but one example, the WHO defines metabolic syndrome as the presence of any one of diabetes mellitus, impaired glucose tolerance, impaired fasting glucose or insulin resistance and two of the following: blood pressure equal to or greater than 140/90 mmHg, dyslipidemia, central obesity, and microalbuminuria. In some embodiments, the metabolic disorder can be selected from the group consisting of: obesity; excess adipose tissue; diabetes; and cardiovascular disease.

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. Described herein are methods and compositions which can provide low toxicity with improved delivery kinetics.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

A carboxylic acid is a carbonyl-bearing functional group having a formula RCOOH where R is aliphatic, heteroaliphatic, alkyl, or heteroalkyl.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynyls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine. A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. Examples of cyclyl groups include, without limitation, cyclopropyl, cyclobutyl, cyclopentyl, cyclopentenyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

The terms “bicyclic” and “tricyclic” refers to fused, bridged, or joined by a single bond polycyclic ring assemblies. As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

As used herein, the term “substituted” refers to independent replacement of one or more of the hydrogen atoms on the substituted moiety with substituents independently selected from, but not limited to, alkyl, alkenyl, heterocycloalkyl, alkoxy, aryloxy, hydroxy, amino, amido, alkylamino, arylamino, cyano, halo, mercapto, nitro, carbonyl, acyl, aryl and heteroaryl groups.

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O— alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example -NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like. The term “alkoxycarbonyl” means —C(O)O-(alkyl), such as —C(═O)OCH₃, —C(═O)OCH₂CH₃, and the like. The term “alkoxyalkyl” means -(alkyl)-O-(alkyl), such as —CH₂OCH₃, —CH₂OCH₂CH₃, and the like. The term “aryloxy” means —O-(aryl), such as —O-phenyl, —O-pyridinyl, and the like. The term “arylalkyl” means -(alkyl)-(aryl), such as benzyl (i.e., —CH₂phenyl), —CH₂-pyrindinyl, and the like. The term “arylalkyloxy” means —O-(alkyl)-(aryl), such as —O-benzyl, —O—CH₂-pyridinyl, and the like. The term “cycloalkyloxy” means —O-(cycloalkyl), such as —O-cyclohexyl, and the like. The term “cycloalkylalkyloxy” means —O-(alkyl)-(cycloalkyl, such as —OCH₂cyclohexyl, and the like. The term “aminoalkoxy” means —O-(alkyl)-NH₂, such as —OCH₂NH₂, —OCH₂CH₂NH₂, and the like. The term “mono- or di-alkylamino” means —NH(alkyl) or —N(alkyl)(alkyl), respectively, such as —NHCH₃, —N(CH₃)₂, and the like. The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like. The term “arylamino” means —NH(aryl), such as —NH-phenyl, —NH-pyridinyl, and the like. The term “arylalkylamino” means —NH-(alkyl)-(aryl), such as —NH-benzyl, —NHCH₂-pyridinyl, and the like. The term “alkylamino” means —NH(alkyl), such as —NHCH₃, —NHCH₂CH₃, and the like. The term “cycloalkylamino” means —NH-(cycloalkyl), such as —NH-cyclohexyl, and the like. The term “cycloalkylalkylamino” —NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane polarized light in different directions). Enantiomers generally have identical physical properties, such as melting points and boiling points, and also have identical spectroscopic properties. Enantiomers can differ from each other with respect to their interaction with plane-polarized light and with respect to biological activity.

The term “racemic mixture”, “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); or (R) and (S) forms). The terms can also refer to enantioselective conversion of one isomer of a racemate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F₍₊₎ and F₍₋₎ (where the sum of F₍₊₎ and F₍₋₎=1). The enantiomeric excess is defined as *F₍₊₎−F₍₋₎* and the percent enantiomeric excess by 100×*F₍₊₎−F₍₋₎*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer”, “substantially resolved enantiomer” “substantially purified enantiomer preparation” are meant to indicate a preparation (e.g. derived from non-optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer”, “resolved enantiomer” and “purified enantiomer preparation” are meant to indicate a preparation (e.g. derived from non-optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereopreferred procedure, followed by separation steps, or a purified enantiomer may be derived from a racemic mixture.

The term “enantioselectivity”, also called the enantiomeric ratio indicated by the symbol “E”, refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

CAGE (Choline And GEranate) is an ionic liquid comprising the cation choline (see, e.g., Structure I) and the anion geranate or geranic acid (see, e.g., Structures II and III). Preparation of CAGE can be, e.g., as described in International Patent Publication WO 2015/066647; which is incorporated by reference herein in its entirety, or as described in the examples herein.

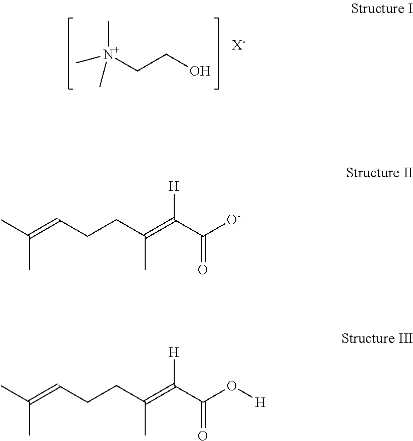

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of any of the aspects, a variant can be a polypeptide having at least 90%, at least 95%, at least 98% or greater sequence homology to one of the reference sequences provided herein and retaining the wild-type activity of that reference sequence, e.g., incretin activity. In some embodiments of any of the aspects, a variant can be a polypeptide having at least 90%, at least 95%, at least 98% or greater sequence homology to one of the naturally-occurring reference sequences provided herein and retaining the wild-type activity of that reference sequence, e.g., incretin activity. In some embodiments of any of the aspects, a variant can be a naturally-occurring polypeptide having at least 90%, at least 95%, at least 98% or greater sequence homology to one of the reference sequences provided herein and retaining the wild-type activity of that reference sequence, e.g., incretin activity.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof, including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., cDNA. Suitable RNA can include, e.g., mRNA.

As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art. One skilled in the art would be able to design further siRNA, shRNA, or miRNA to target the nucleic acid sequence of a target gene or gene product (e.g., mRNA), e.g., using publically available design tools. siRNA, shRNA, or miRNA is commonly made using companies such as Dharmacon (Layfayette, CO) or Sigma Aldrich (St. Louis, MO).

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2-O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Cane Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO]mCH3, O(CH2).nOCH3, O(CH2)nNH2, O(CH2)nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), athioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA:nuclease complex binds a target polynucleotide sequence having a protospacer adjacent motif (“PAM”) and a protospacer, which is a sequence complementary to a portion of the gRNA. The recognition and binding of the target polynucleotide by the gRNA:nuclease complex induces cleavage of the target.

Any CRISPR-associated nuclease can be used in the system and methods of the invention. CRISPR nuclease systems are known to those of skill in the art, e.g. Cas9, Cas12, Cas12a, or the like, see patents/applications U.S. Pat. No. 8,993,233, US 2015/0291965, US 2016/0175462, US 2015/0020223, US 2014/0179770, U.S. Pat. Nos. 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; U.S. Pat. Nos. 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a recombinant vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a condition or disease described herein. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “effective amount” means an amount of a composition sufficient to provide at least some amelioration of the symptoms associated with the condition. In one embodiment, the “effective amount” means an amount of a composition would decrease the markers or symptoms of the condition in a subject having the condition.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean+1%.

As used herein, the term “comprising” or “comprises” is used in reference to methods and compositions, and respective component(s) thereof, that are essential to the invention, yet open to the inclusion of unspecified elements, whether essential or not. As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th}ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A composition comprising at least one ionic liquid comprising:
   an anion which is at least one of:
      a) a carboxylic acid which is not a fatty acid;
      b) a carboxylic acid comprising an aliphatic chain of no more than 4 carbons;
      c) an aromatic anion; and/or
      d) an anion with a LogP of less than 1.0; and
   a cation comprising a quaternary ammonium.
2. The composition of any of the preceding paragraphs, wherein the anion has a LogP of less than 1.0 and is:
   a. a carboxylic acid which is not a fatty acid;
   b. carboxylic acid comprising an aliphatic chain of no more than 4 carbons; or
   c. an aromatic anion.
3. The composition of any of the preceding paragraphs, wherein the fatty acid comprises an aliphatic chain of no more than 3 carbons.
4. The composition of any of the preceding paragraphs, wherein the anion comprises only one carboxylic acid group (e.g., R—COOH group).
5. The composition of any of the preceding paragraphs, wherein the anion is selected from the group consisting of:
   glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid; isovaleric acid; hydrocinnaminic acid; 4-phenolsulfonic acid; phenyl phosphoric acid; and biphenyl-3-carboxylic acid.
6. The composition of any of the preceding paragraphs, wherein the cation has a molar mass equal to or greater than choline.
7. The composition of any of the preceding paragraphs, wherein the quaternary ammonium has the structure of NR₄⁺ and at least one R group comprises a hydroxy group.
8. The composition of any of the preceding paragraphs, wherein the quaternary ammonium has the structure of NR₄⁺ and only one R group comprises a hydroxy group.
9. The composition of any of the preceding paragraphs, wherein the cation is C1, C6, or C7.
10. The composition of any of the preceding paragraphs, wherein the ionic liquid comprises a ratio of cation to anion of from about 2:1 to about 1:1.
11. The composition of any of the preceding paragraphs, wherein the ionic liquid comprises a ratio of cation to anion of about 2:1.
12. The composition of any of the preceding paragraphs, wherein the ionic liquid has a cation:anion ratio of less than 1:1.
13. The composition of any of the preceding paragraphs, wherein the ionic liquid has a cation:anion ratio with an excess of cation.
14. The composition of any of the preceding paragraphs, further comprising at least one active compound in combination with the at least one ionic liquid.
15. The composition of any of the preceding paragraphs, wherein the active compound comprises a polypeptide.
16. The composition of paragraph 15, wherein the polypeptide is an antibody or antibody reagent.
17. The composition of any of paragraphs 15-16, wherein the active compound has a molecular weight of greater than 450.
18. The composition of any of paragraphs 15-16, wherein the active compound has a molecular weight of greater than 500.
19. The composition of any of paragraphs 15-18, wherein the anion has a LogP of less than 1.0 and is:
   a. a carboxylic acid which is not a fatty acid; or
   b. a carboxylic acid comprising an aliphatic chain of no more than 4 carbons.
20. The composition of any of the preceding paragraphs, wherein the active compound comprises a nucleic acid.
21. The composition of paragraph 20, wherein the nucleic acid is an inhibitory nucleic acid.
22. The composition of paragraph 21, wherein the nucleic acid is a siRNA.
23. The composition of any of paragraphs 20-22, wherein the anion has a LogP of less than 1.0 and is:
   a. a carboxylic acid which is not a fatty acid; or
   b. a carboxylic acid comprising an aliphatic chain of no more than 4 carbons; and/or
   c. an aromatic anion.
24. The composition of any of the preceding paragraphs, wherein the ionic liquid is at a concentration of at least 0.1% w/v.
25. The composition of any of the preceding paragraphs, wherein the ionic liquid is at a concentration of from about 10 to about 70% w/v.
26. The composition of any of the preceding paragraphs, wherein the ionic liquid is at a concentration of from about 30 to about 50% w/v.
27. The composition of any of the preceding paragraphs, wherein the ionic liquid is at a concentration of from about 30 to about 40% w/v.
28. The composition of any of the preceding paragraphs, wherein the composition is formulated for administration transdermally, to a mucus membrane, orally, subcutaneously, intradermally, parenterally, intratumorally, or intravenously.
29. The composition of paragraph 28, wherein the composition is formulated for transdermal administration.
30. The composition of paragraph 28, wherein the mucus membrane is nasal, oral, or vaginal.
31. The composition of any of the preceding paragraphs, wherein the active compound is provided at a dosage of 1-40 mg/kg.
32. The composition of any of the preceding paragraphs, further comprising at least one non-ionic surfactant.
33. The composition of any of the preceding paragraphs, further comprising a pharmaceutically acceptable carrier.
34. The composition of any of the preceding paragraphs, wherein the composition is provided in a degradable capsule.
35. The composition of any of the preceding paragraphs, wherein the composition is an admixture.
36. The composition of any of the preceding paragraphs, wherein the composition is provided in one or more nanoparticles.
37. The composition of any of the preceding paragraphs, comprising one or more nanoparticles comprising the active compound, the nanoparticles in solution or suspension in a composition comprising the ionic liquid.
38. A method of administering at least one active compound, the method comprising administering a composition of any of paragraphs 14-37.
39. The method of paragraph 38, wherein the composition is administered once.
40. The method of any of paragraphs 38-39, wherein the composition is administered in multiple doses.

Some embodiments of the technology described herein can be defined according to any of the following numbered paragraphs:
1. A composition comprising at least one ionic liquid comprising:
   an anion which is at least one of:
      a) a carboxylic acid which is not a fatty acid;
      b) a carboxylic acid comprising an aliphatic chain of no more than 4 carbons;
      c) an aromatic anion; and/or
      d) an anion with a LogP of less than 1.0; and
   a cation comprising a quaternary ammonium.
2. The composition of any of the preceding paragraphs, wherein the anion has a LogP of less than 1.0 and is:
   a. a carboxylic acid which is not a fatty acid;
   b. carboxylic acid comprising an aliphatic chain of no more than 4 carbons; or
   c. an aromatic anion.
3. The composition of any of the preceding paragraphs, wherein the fatty acid comprises an aliphatic chain of no more than 3 carbons.
4. The composition of any of the preceding paragraphs, wherein the anion comprises only one carboxylic acid group (e.g., R—COOH group).
5. The composition of any of the preceding paragraphs, wherein the anion is selected from the group consisting of:
   geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic
6. The composition of any of the preceding paragraphs, wherein the anion is selected from the group consisting of:
   glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid.
7. The composition of any of the preceding paragraphs, wherein the cation has a molar mass equal to or greater than choline.
8. The composition of any of the preceding paragraphs, wherein the quaternary ammonium has the structure of NR₄⁺ and at least one R group comprises a hydroxy group.
9. The composition of any of the preceding paragraphs, wherein the quaternary ammonium has the structure of NR₄⁺ and only one R group comprises a hydroxy group.
10. The composition of any of the preceding paragraphs, wherein the cation is choline, C1, C6, or C7.
11. The composition of any of the preceding paragraphs, wherein the cation is choline.
12. The composition of any of the preceding paragraphs, wherein the cation is C1, C6, or C7.
13. The composition of any of the preceding paragraphs, wherein the ionic liquid comprises a ratio of cation to anion of from about 2:1 to about 1:1.
14. The composition of any of the preceding paragraphs, wherein the ionic liquid comprises a ratio of cation to anion of about 2:1.
15. The composition of any of the preceding paragraphs, wherein the ionic liquid has a cation:anion ratio of less than 1:1.
16. The composition of any of the preceding paragraphs, wherein the ionic liquid has a cation:anion ratio with an excess of cation.
17. The composition of any of the preceding paragraphs, comprising a first ionic liquid and at least a second ionic liquid.
18. The composition of paragraph 17, wherein each ionic liquid has a choline cation.
19. The composition of any of paragraphs 17-18, wherein the first ionic liquid and the second ionic liquid each comprise a different anion.
20. The composition of paragraph 19, wherein the first ionic liquid and the second ionic liquid each comprise a different anion selected from:
   geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid.
21. The composition of any of paragraphs 17-20, wherein the first ionic liquid has a geranic acid anion and the second ionic liquid has a phenylpropanoic acid anion.
22. The composition of any of paragraphs 17-21, wherein the first ionic liquid is choline and geranic acid (CAGE).
23. The composition of any of paragraphs 17-22, wherein the second ionic liquid is choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).
24. The composition of any of paragraphs 17-21, wherein the first and second ionic liquids are different ionic liquids selected from the group consisting of: choline and geranic acid (CAGE); choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).
25. The composition of any of paragraphs 17-21, wherein the first ionic liquid is selected from the group consisting of: choline and geranic acid (CAGE); choline and dimethylacrylic acid (CADA); and choline and choline and biphenyl-3-carboxylic acid (CABA); and the second ionic liquid is selected from the group consisting of: isovaleric acid (CAVA); and choline and phenylpropanoic acid (CAPA).
26. The composition of any of paragraphs 17-22, wherein the first ionic liquid is choline and geranic acid (CAGE) and the second ionic liquid is choline and phenylpropanoic acid (CAPA).
27. The composition of any of the preceding paragraphs, further comprising at least one active compound in combination with the at least one ionic liquid.
28. The composition of any of the preceding paragraphs, wherein the active compound comprises a polypeptide.
29. The composition of paragraph 28, wherein the polypeptide is an antibody or antibody reagent.
30. The composition of any of paragraphs 28-29, wherein the active compound has a molecular weight of greater than 450.
31. The composition of any of paragraphs 28-30, wherein the active compound has a molecular weight of greater than 500.
32. The composition of any of paragraphs 28-31, wherein the anion has a LogP of less than 1.0 and is:
   a. a carboxylic acid which is not a fatty acid; or
   b. a carboxylic acid comprising an aliphatic chain of no more than 4 carbons.
33. The composition of any of the preceding paragraphs, wherein the active compound comprises a nucleic acid.
34. The composition of paragraph 33, wherein the nucleic acid is an inhibitory nucleic acid.
35. The composition of paragraph 34, wherein the nucleic acid is a siRNA.
36. The composition of any of paragraphs 34-35, wherein the inhibitory nucleic acid is a NFKBIZ, TNFalpha, and/or IL-17 inhibitory nucleic acid.
37. The composition of any of paragraphs 33-36, wherein the anion has a LogP of less than 1.0 and is:
   a. a carboxylic acid which is not a fatty acid; or
   b. a carboxylic acid comprising an aliphatic chain of no more than 4 carbons; and/or
   c. an aromatic anion.
38. The composition of any of the preceding paragraphs, wherein the ionic liquid is at a concentration of at least 0.1% w/v.
39. The composition of any of the preceding paragraphs, wherein the ionic liquid is at a concentration of from about 10 to about 70% w/v.
40. The composition of any of the preceding paragraphs, wherein the ionic liquid is at a concentration of from about 30 to about 50% w/v.
41. The composition of any of the preceding paragraphs, wherein the ionic liquid is at a concentration of from about 30 to about 40% w/v.
42. The composition of any of the preceding paragraphs, wherein the composition is formulated for administration transdermally, to a mucus membrane, orally, subcutaneously, intradermally, parenterally, intratumorally, or intravenously.
43. The composition of paragraph 42, wherein the composition is formulated for transdermal administration.
44. The composition of paragraph 42, wherein the mucus membrane is nasal, oral, or vaginal.
45. The composition of any of the preceding paragraphs, wherein the active compound is provided at a dosage of 1-40 mg/kg.
46. The composition of any of the preceding paragraphs, further comprising at least one non-ionic surfactant.
47. The composition of any of the preceding paragraphs, further comprising a pharmaceutically acceptable carrier.
48. The composition of any of the preceding paragraphs, wherein the composition is provided in a degradable capsule.
49. The composition of any of the preceding paragraphs, wherein the composition is an admixture.
50. The composition of any of the preceding paragraphs, wherein the composition is provided in one or more nanoparticles.
51. The composition of any of the preceding paragraphs, comprising one or more nanoparticles comprising the active compound, the nanoparticles in solution or suspension in a composition comprising the ionic liquid.
52. A method of administering at least one active compound to a subject, the method comprising administering a composition of any of paragraphs 27-51.
53. The method of paragraph 52, wherein the composition is administered once.
54. The method of any of paragraphs 52-53, wherein the composition is administered in multiple doses.
55. The method of any of paragraphs 52-54, wherein the administering is transdermally, to a mucus membrane, orally, subcutaneously, intradermally, parenterally, intratumorally, or intravenously
56. The method of any of paragraphs 52-55, wherein the composition comprises a NFKBIZ, TNFalpha, and/or IL-17 inhibitory nucleic acid and the subject is in need of treatment for an inflammatory condition.
57. A method of treating an inflammatory condition in a subject in need thereof, the method comprising administering a composition of any of paragraphs 36-51 to the subject.
58. The method of any of paragraphs 56-57, wherein the administration is topical.
59. The method of any of paragraphs 56-58, wherein the inflammatory condition is psoriasis.
60. A composition of any of paragraphs 27-51, for use in a method of administering at least one active compound to a subject.
61. The composition of paragraph 60, wherein the composition is administered once.
62. The composition of paragraph 60, wherein the composition is administered in multiple doses.
63. The composition of any of paragraphs 60-62, wherein the administering is transdermally, to a mucus membrane, orally, subcutaneously, intradermally, parenterally, intratumorally, or intravenously
64. The composition of any of paragraphs 60-63, wherein the composition comprises a NFKBIZ, TNFalpha, and/or IL-17 inhibitory nucleic acid and the subject is in need of treatment for an inflammatory condition.
65. A composition of any of paragraphs 36-51 for use in a method of treating an inflammatory condition in a subject in need thereof.
66. The composition of any of paragraphs 64-65, wherein the administration is topical.
67. The composition of any of paragraphs 64-66, wherein the inflammatory condition is psoriasis.

### EXAMPLES

### Example 1: Ionic Liquids for Oral Monoclonal Antibody Delivery

Monoclonal antibodies (mAbs) are currently used for the treatment for numerous conditions including cancer, psoriasis, arthritis, and atopic dermatitis, among others. All mAbs are currently administered by either intravenous or subcutaneous injections. Described herein is the use of a novel ionic liquid, choline and glycolate (glycolic acid) (CGLY), as a platform for oral administration of therapeutic antibodies. CGLY maintained the stability and structure of TNFα antibody. CGLY significantly enhanced paracellular transport of TNFα antibody in vitro. CGLY also reduced the viscosity of the intestinal mucus, another key barrier for antibody transport. In vivo results in rats demonstrate that CGLY effectively delivers TNFα antibody into the intestinal mucosa as well as systemic circulation. One week repeat dose study followed by histology and serum biochemistry analysis indicated that CGLY is well tolerated by rats. Overall, this work illustrates the benefits of using choline-based ionic liquids as an oral delivery platform for local as well as systemic delivery of therapeutic antibodies.

Therapeutic monoclonal antibodies (mAbs) are by far the largest class of protein-based therapeutic agents^{[1, 2]}. More than 50 mAb-based products have been approved as products and over 500 mAb-based therapies in clinical development^{[3]}. Antibodies are used to treat a wide variety of diseases including cancer, infection, inflammation and autoimmune diseases^{[1, 4]}. However, mAbs are delivered as intravenous infusion or subcutaneous injection dosage forms, which are associated with adverse effects such as systemic inflammatory response, infusion reactions and low patience compliance due to pain and needle phobia^{[5-7]}. Oral administration of mAbs offers potential advantages over injections owing to its simplicity of administration, high patient acceptability and low manufacturing cost. In addition to offering a potential means for non-invasive systemic administration, oral administration also offers a means to deliver antibodies locally into the gastrointestinal tract for the treatment of local diseases such as inflammatory bowel disease^{[8-10]}. Nonetheless, as with all the oral delivery of proteins, a multitude of gastrointestinal barriers collectively limit protein drug absorption^{[11, 12]}. This has motivated efforts to develop oral antibody formulations that can achieve therapeutic outcomes in a more effective manner. For example, recombinant antibodies against tumor necrosis factor (TNF) are being developed for treating gastrointestinal infections and inflammatory bowel disease^{[13-15]}. With recombinant moieties, the antibodies showed improved tolerance to intestinal proteases and resist degradation. In addition, an engineered anti-TNF antibody fragments also demonstrated improved permeation into the diseased tissue within the GI tract^{[14]}.

Described herein is the investigation of the potential of choline-based ILs for oral IgG delivery. To achieve this, choline-glycolate (CGLY) ionic liquid was prepared and assessed with respect to antibody stability, in vitro transport and in vivo uptake.

Results

Physicochemical Characterization of IgG-CGLY Variant Formulations

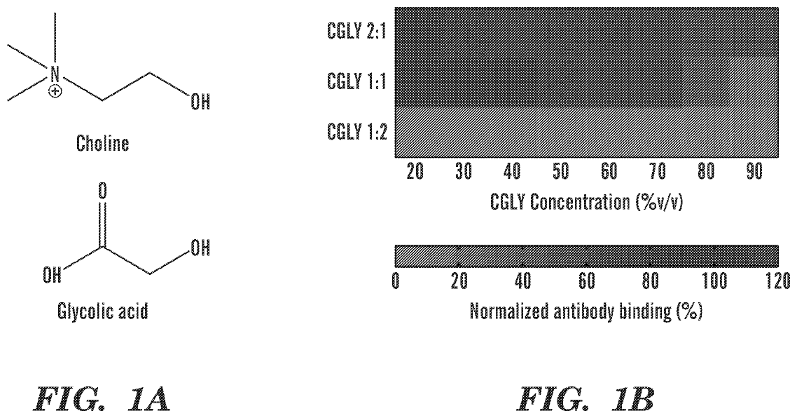

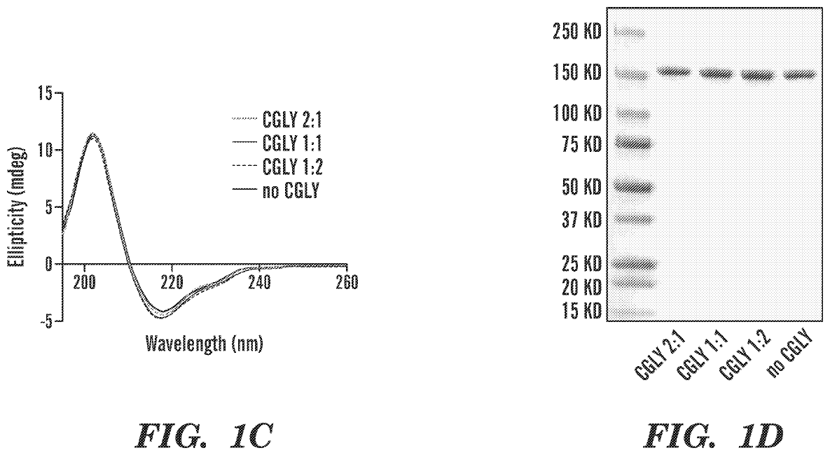

Initial studies were performed to assess the role of ion stoichiometry in CGLY on the compatibility with IgG antibody. Three variants of CGLY were synthesized with choline:glycolic acid molar ratios of 2:1, 1:1, and 1:2 (FIG. 1A). A model IgG antibody, anti-human TNF-α mouse IgG1 (clone MAb11), was dissolved at a concentration of 0.1 mg mL⁻¹ in CGLY variants diluted in saline over a range between 20-90% v/v. The IgG antibody dissolved completely in all CGLY variants and concentrations, no precipitation was observed. After 1 h incubation and 48 h dialysis at room temperature, the antibody samples were evaluated based on their antigen binding capacity using ELISA (FIG. 1B). IgG-CGLY formulations with CGLY_{2:1} and CGLY_{1:1} showed negligible impact on the inherent binding capability of TNF-α IgG1 at CGLY concentrations up to 60% and 70% v/v respectively. On the other hand, the IgG antibody samples isolated from CGLY_{1:2} induced a reduction of binding efficiency at concentration range from 20-90% v/v.

To further elucidate the impact of CGLY on IgG antibody, circular dichroism (CD) and SDS-PAGE analysis were performed. Since the presence of CGLY generates significant background CD noise, the IgG-CGLY samples were dialyzed for 48 h at room temperature prior to CD measurement. The far-UV wavelength spectra of anti-human TNF-α IgG showed no difference in the shape or degree of ellipticity compared to pristine IgG (FIG. 1C). All CD spectra showed a minimum at 218 nm, which is a typical manifestation for β-sheets, the predominant secondary structure of IgGs^{[30, 31]}. The result indicates that the structural conformation of the IgG is retained after being exposed to CGLY variants. In parallel, SDS-PAGE was also used to assess the impact of CGLY on anti-human TNF-α IgG with a particular eye on potential IgG aggregation^{[32]}. Without the presence of CGLY, the model IgG appears as a single band at a molecular weight ˜150 kDa (FIG. 1D). The IgG from all CGLY variant groups are also identified in the same band location. No other bands appeared below or above the 150 kDa band suggesting that there is no detectable fragmentation or aggregation of the antibody from formulations with CGLY^{[32, 33]}. Taken together, the antibody characterization results from ELISA, CD spectroscopy and SDS PAGE indicated that CGLY_{2:1} and CGLY_{1:1} had minimal effects of the conformation or the antibody aggregation.

Impact of CGLY on Caco-2 Cell Viability and IgG Transport

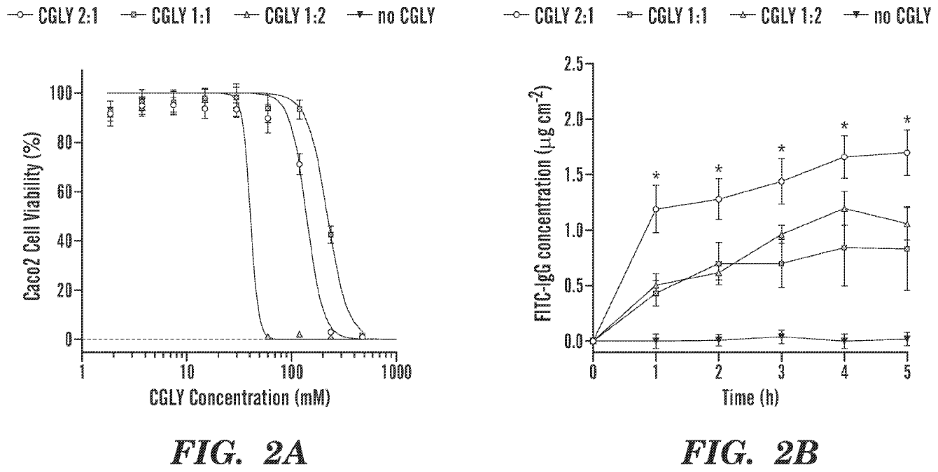

Caco-2 cells were highly tolerant to CGLY_{2:1} and CGLY_{1:1}, no adverse effect on cell proliferation was observed until high concentration >100 mM, whereas CGLY_{1:2} diminished the cell viability at considerably lower concentrations (FIG. 2A). The IC50 of CGLY_{2:1}, CGLY_{1:1} and CGLY_{1:2} were approximated to 140.4 mM, 223.3 mM, and 40.78 mM respectively.

The ability of CGLY to enhance trans-epithelial transport was studied using fluorescein isothiocyanate labeled (FITC)-IgG across Caco-2 monolayer. These studies were performed using 30 mM of CGLY which was well below the IC50 of all CGLY variants. Throughout the 5 h-long study, FITC-IgG transport progressively increased with time in all CGLY groups while no FITC-IgG transport was detectable from no CGLY control transwells (FIG. 2B). Specifically, CGLY_{2:1} showed the highest significant transport of IgG among the CGLY variants across all time points. At the end of the study, the mean IgG transport in monolayers treated with CGLY_{2:1} was 1.70 μg cm⁻² which was over 2-fold higher than CGLY_{1:1} (0.83 μg cm⁻²) and 1.6-fold higher than CGLY_{1:2} (1.06 μg cm⁻²) treated cells.

By taking into account the results from IgG antibody-CGLY characterization and the Caco-2 cells response to CGLY, CGLY_{2:1} stood out as the optimal ionic liquid for IgG antibody delivery among the CGLY variants studied. Therefore, CGLY_{2:1} was selected for further in vitro and in vivo investigations hereafter.

Detailed Analysis of CGLY_{2:1} Mediated Antibody Transport Across Caco-2 Intestinal Cells

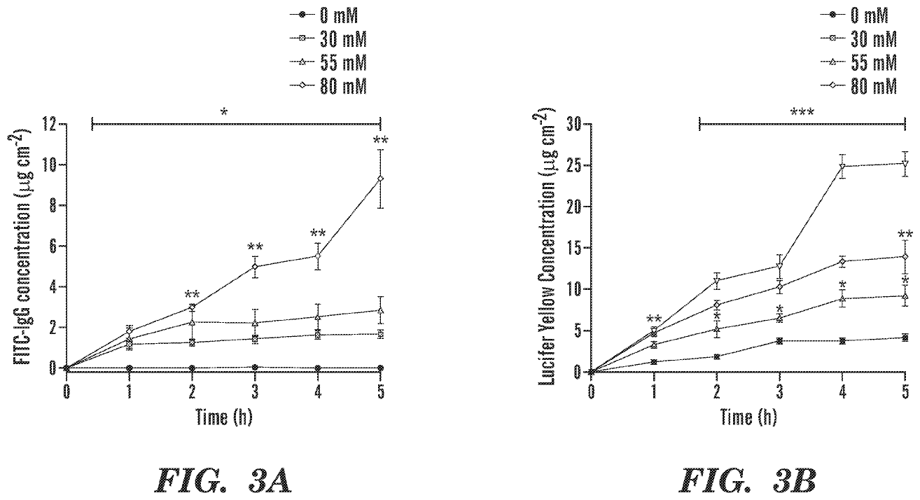

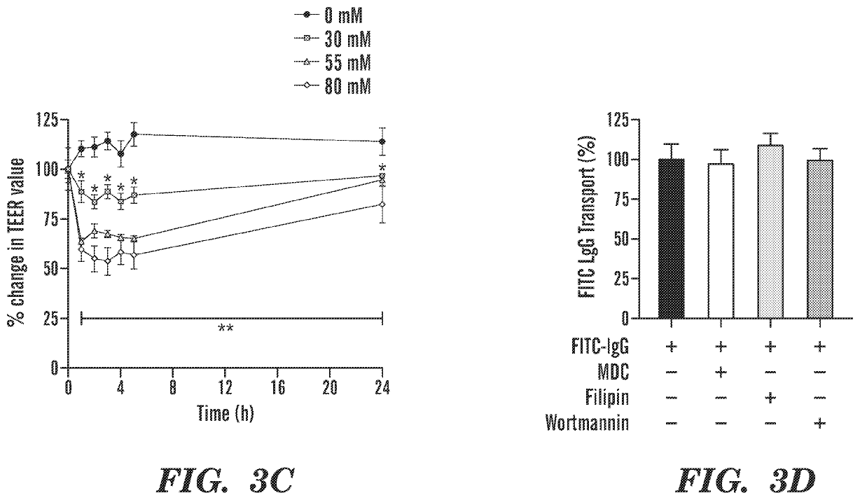

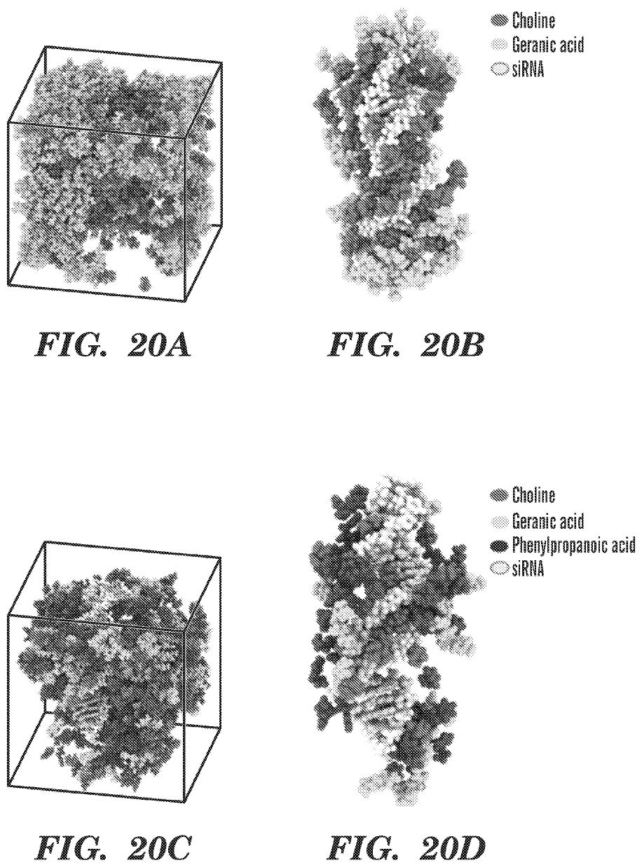

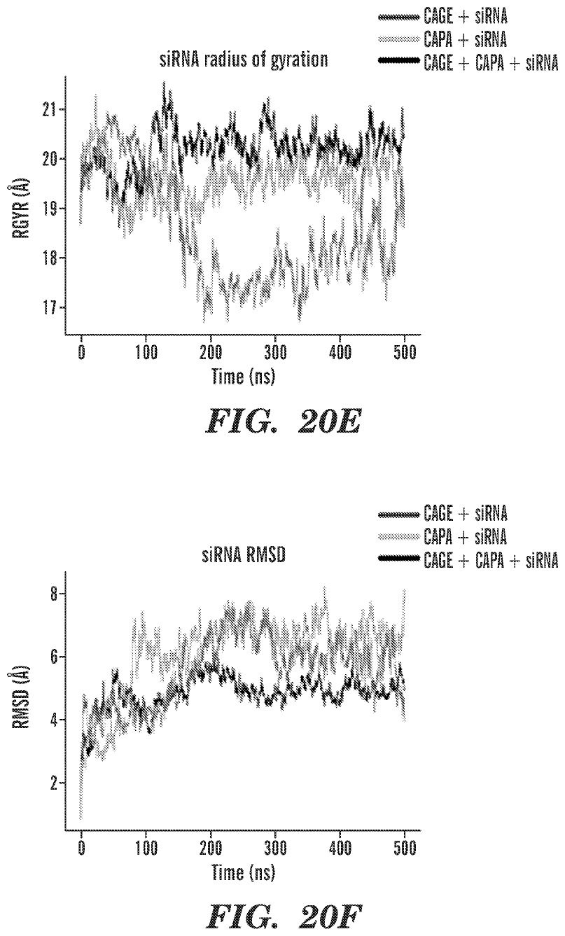

CGLY_{2:1} enhanced trans-epithelial transport of FITC-IgG across Caco-2 monolayer in a concentration-dependent manner (FIG. 3A). As the CGLY_{2:1} concentration increased from 30 mM to 80 mM, the amount transported increased from 1.70 μg cm⁻² to 9.32 μg cm⁻². Meanwhile, it is noteworthy to point out that the FITC-IgG transport was undetectable in the absence of CGLY_{2:1}. These results were consistent with the transport assessed from confocal images of Caco-2 cells (FIG. 20). The fluorescence images of the cells at the end of the 5 h study clearly illustrated higher uptake of FITC-IgG by Caco-2 cells with increasing concentration of CGLY_{2:1} compared to control wells.

Paracellular and transcellular are the major routes involved in the transport of peptides and proteins across intestinal epithelia. To investigate the mechanism of CGLY_{2:1}-mediated IgG transport across Caco-2 monolayer, role of both paracellular and transcellular transport was investigated. First, the paracellular route was assessed via the transport of Lucifer yellow, a paracellular transport marker^{[34]}. The amount of transported Lucifer yellow was drastically improved across all time points on cells treated with 30-80 mM CGLY_{2:1} (FIG. 3B). At the lowest range of 30 mM CGLY_{2:1}, Lucifer yellow transport was enhanced by ˜2 fold. At 80 mM CGLY_{2:1} concentration, the transport of Lucifer yellow was enhanced by 4-6 fold at various time points. In a parallel experiment, trans epithelial electrical resistance (TEER) measurement of Caco-2 transwells with varying concentrations of CGLY2:1 was conducted to assess the tight junction integrity of Caco-2 monolayers and to further validate the paracellular involvement in CGLY_{2:1}-assisted transport. For untreated wells, TEER measurement showed slight increase within 15% range until the end of study at 24 h, which is consistent with previous literatures^{[23, 35]}. With the addition of 30 mM CGLY_{2:1}, TEER value decreased by 11% in 1 h and remained within 11-16% reduction in the first 5 h. The TEER drop by CGLY_{2:1}, however, was evidently transient and the cells recovered 96% of their tight junction integrity in 24 h. By increasing CGLY_{2:1} concentration, the extent of TEER reduction was further augmented. Approximately 34% TEER drop was observed with 55 mM CGLY_{2:1} and 45% drop with 80 mM CGLY_{2:1} at 1-5 h of the study, indicating opening of tight junctions. Nonetheless, the CGLY_{2:1}-induced TEER reduction still exhibited transient behavior and in 24 h the cells regained 94% and 82% of initial TEER values with 55 mM and 80 mM CGLY_{2:1}, respectively. The decline and recovery of TEER measurements when the cells were treated by 30-80 mM CGLY_{2:1} indicate that CGLY_{2:1} can temporarily open the intestinal tight junctions and promote IgG transport across the intestinal epithelial barrier. Together, the increasing of Lucifer yellow transport and TEER value reduction with the presence of increasing CGLY_{2:1} confirmed paracellular transport characteristic of CGLY_{2:1.}

Contribution of transcellular pathway to IgG transport was studied by assessing the impact of transcytosis inhibitors including monodansylcadaverine (MDC; inhibitor of clathrin-mediated endocytosis), filipin (inhibitor of caveolar-mediated endocytosis), and wortmannin (inhibitor of phosphatidylinositol 3 kinase, involved in micropinocytosis)^{[36]}. The cumulative transport of FITC-IgG after 24 h incubation did not show a notable difference between any of the inhibitor-treated cells compared to no inhibitor control (FIG. 3D). The findings suggested that the improved delivery of FITC-IgG through Caco2 transwell by CGLY_{2:1} was not primarily assisted through transcellular transport.

Effect of CGLY₂-1 on Mucus Viscosity

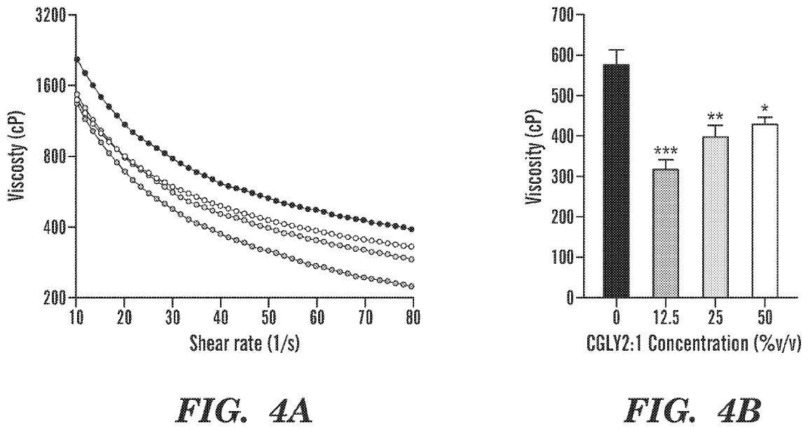

Intestinal mucus is among the critical components of the gut barrier^{[12]}. To investigate the effect of CGLY_{2:1} on porcine small intestinal mucus (PIM), the rheology of PIM treated with CGLY_{2:1} was assessed. FIG. 4A illustrates shear-thinning profiles of PIM samples after incubating with 0-50% v/v of CGLY_{2:1}. Compared to untreated PIM, the viscosity of the mucus treated with CGLY_{2:1} showed a notable drop throughout the entire measured shear range. For instance, at a shear rate of 49.87 l/s, the mean viscosity of untreated PIM was measured as 576.8 cP, a value comparable to the previously reported literatures^{[37, 38]} (FIG. 4B). The addition of 12.5, 25 and 50% v/v of CGLY_{2:1} significantly decreased the mucus viscosity to 317.9, 398.0 and 429.6 cP respectively. The ability of CGLY_{2:1} to reduce mucus viscosity may facilitate antibody delivery to the intestinal epithelia.

In Vivo Local and Systemic Antibody Delivery of IgG by CGLY_{2:1}

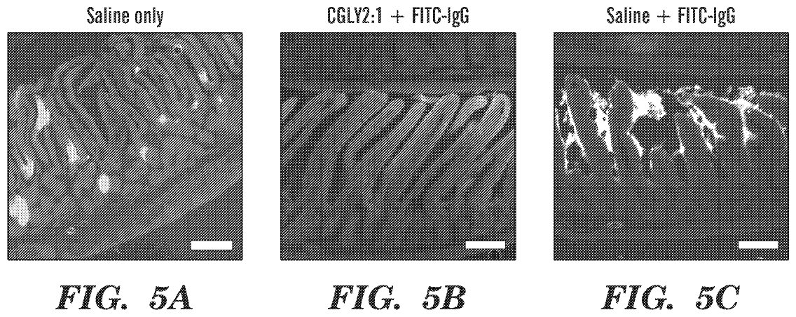

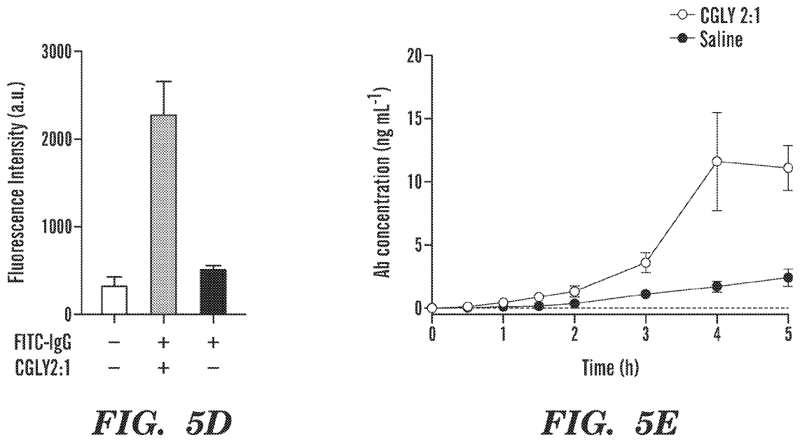

FITC-IgG formulated in CGLY_{2:1} was injected intrajejunally in Wistar rats (1 mg mL⁻¹ of FITC-IgG in 50% v/v CGLY_{2:1}). Control rats received equivalent saline injection with or without FITC-IgG. After 2 h, the jejunal tissues were harvested and cryosections were prepared for imaging (FIGS. 5A-5C) and the fluorescence signal of FITC-IgG per unit area on intestinal villi were quantified (FIG. 5D). There was a significant difference of FITC-IgG signal in the intestinal mucosa between the treatment groups. The jejunal tissue in the CGLY_{2:1}-treated group showed prominent signal of FITC-IgG in the intestinal villi (FIG. 5B) and the enumerated fluorescence signal was over 4.5-fold compared to no-CGLY_{2:1} control (FIG. 5D). On the other hand, for control group with FITC-IgG in saline, the FITC signal on the villi was not significant compared to negative control. The signals from FITC-IgG are rather strictly located on the exterior of the villi, i.e. mucus layer (FIG. 5C), which indicate that the transport of IgG alone is greatly compromised by the mucus barrier. The findings demonstrate that CGLY_{2:1} effectively enhanced IgG permeation through intestinal mucus and epithelial layers.

In a parallel study, the advantage of using CGLY_{2:1} to enhance IgG absorption was determined by measuring plasma IgG levels (FIG. 5E). For the study, anti-human TNF-α IgG monoclonal antibody was used as the model antibody and administered at 200 μg kg⁻¹ with or without CGLY_{2:1} via intrajejunal injection. IgG concentration gradually increased in the first 2 h after injection. At 3-5 h of the study, a prominent enhancement in IgG concentration was observed from CGLY_{2:1}-treated group. Specifically, the IgG concentration CGLY_{2:1}-treated group was 5-fold higher than the control by the end of the study. Together, the superior local FITC-IgG and plasma IgG concentration results indicate that CGLY_{2:1} enabled penetration and transfer of IgG through the villi into bloodstream. More importantly, since the IgG concentration in plasma was detected by ELISA, the transported IgG was functionally preserved. Given the long circulation half-life of antibodies, it is contemplated that repeat oral administrations of IgG can continuously increase blood concentrations and achieve much higher concentrations.

In Vivo Toxicity Evaluation of CGLY_{2:1}

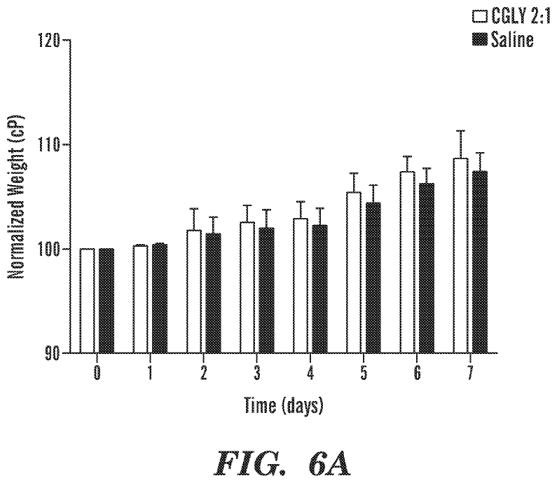

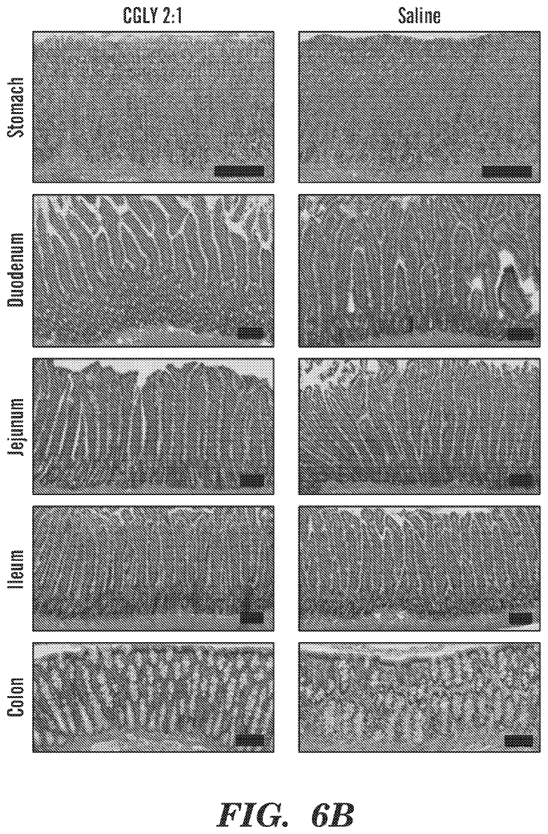

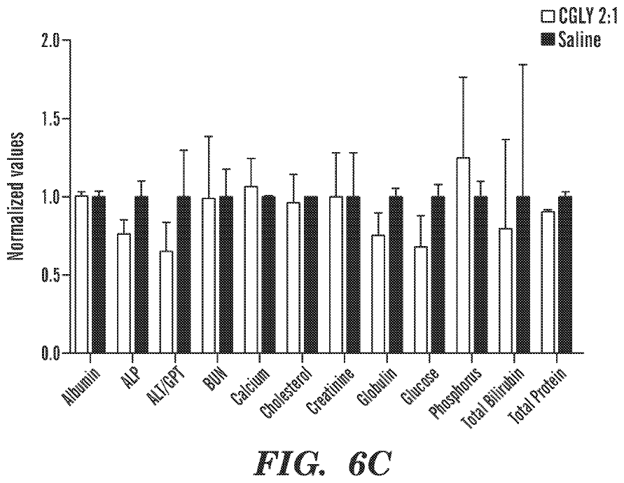

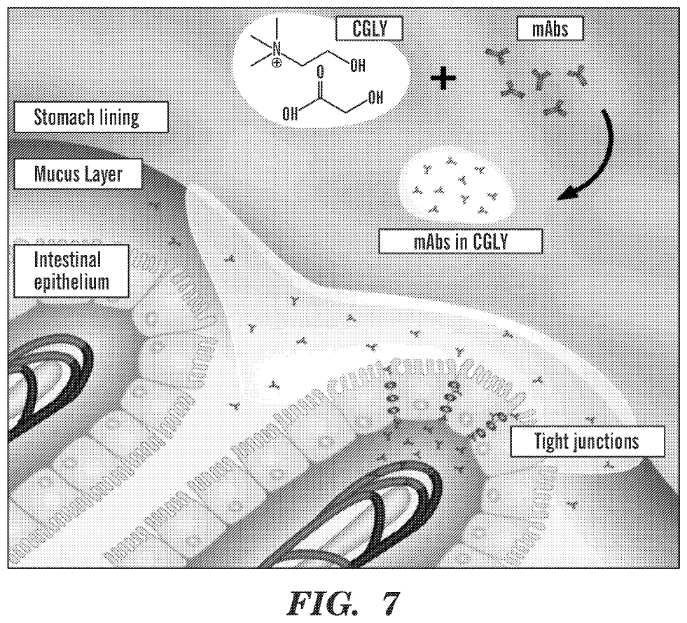

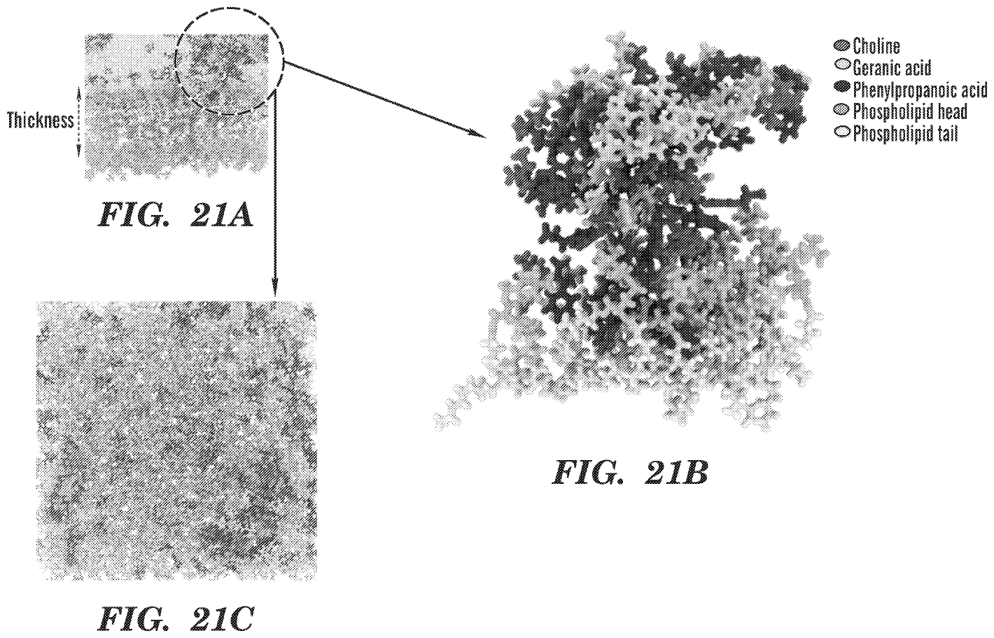

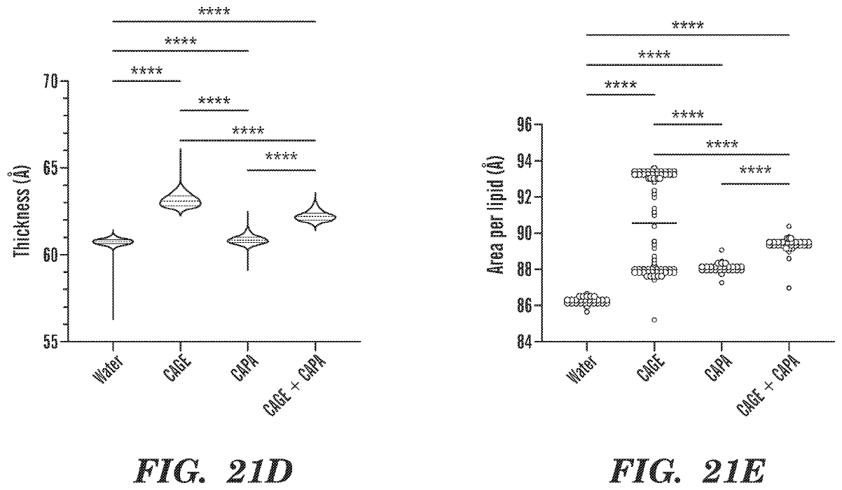

The toxicity of CGLY_{2:1} was evaluated with adult male Wistar rats. CGLY_{2:1} was orally administered once daily for 7 consecutive days at a dose of 625 mg/kg. Rats administered with saline were used as the negative control group. During the study, rats administered with CGLY_{2:1} maintained similar body weight compared to rats administered with saline and all rats showed steady increase in body weight (FIG. 6A). There were also no physiological symptoms such as lethargy, diarrhea hunched posture, or unkempt fur observed in both groups. On day 7, rats were sacrificed, blood samples were collected for metabolic panel analysis, major organs and gastrointestinal (GI) tissues were retrieved from the rats and stained with hematoxylin and eosin (H&E). The tissue sections of the stomach, small intestine (duodenum, jejunum, and ileum) and colon from CGLY_{2:1}-treated group showed unaltered gastric and intestinal mucosal epithelial structures including size and number of crypt and villus, and mucosal thickness compared to saline control group (FIG. 6B). There was no infiltration of immune cells such as neutrophils, lymphocytes, or macrophages into the mucosa, representing no sign of tissue inflammation. No hemorrhage appeared in H&E staining of major organs and no differences were detected between CGLY_{2:1}-treated and saline control group. (FIG. 21) A comprehensive blood chemistry panel analysis revealed no significant differences between the two groups (FIG. 6C), indicating that CGLY_{2:1} did not impose observable adverse effects on liver and kidney functions in the rats. The in vivo toxicity studies of CGLY_{2:1} showed no effect on the rat body weight, blood metabolic panel or histopathologic changes, indicate that CGLY_{2:1} is safe for oral administration in rat models.

### CONCLUSION

Choline glycolate ILs with varying ion stoichiometry were synthesized. Among the CGLY variants, CGLY_{2:1}, with 2:1 molar ratio of choline to glycolic acid, showed excellent cell compatibility, IgG integrity preservation and performed the best in transporting IgG antibody in vitro. Further investigation of CGLY_{2:1} revealed that CGLY_{2:1} can temporarily disturb intestinal tight junction integrity and CGLY_{2:1}-enhanced IgG transport across Caco-2 cells was via paracellular route. CGLY_{2:1} is also capable to reduce mucus viscosity. Intrajejunal administration of IgG in CGLY_{2:1} substantially improved antibody absorption into rat intestinal villi and raised the plasma concentration of model monoclonal antibody up to 5 fold compared to the negative control. In addition, CGLY_{2:1} treatment showed no adverse effects on rat body weight, GI tract histological alteration or blood comprehensive metabolic panel. Overall, this report demonstrates the promise and strength of CGLY_{2:1} is an oral delivery vehicle which can effectively improve both local and systemic bioavailability of IgG antibody with excellent biocompatibility.

Experimental Section

Materials: Glycolic acid, choline bicarbonate, dimethyl sulfoxide (DMSO), FITC-labeled immunoglobulin G from human serum (FITC-IgG, 20 mg mL⁻¹), hematoxylin and eosin solutions were purchased from Sigma-Aldrich (St. Louis, MO, USA). LEAF™ Purified anti-human TNF-α mouse IgG1 (clone Mab11), Recombinant Human TNF-α, ELISA coating buffer, HRP-conjugated goat anti-mouse IgG (clone poly4053), and TMB substrate were purchased from Biolegend (San Diego, CA, USA). 10 mM Sodium Phosphate Buffer, pH=7.4 was obtained from Boston BioProducts (Ashland, MA, USA) and 0.9% sterile saline solution was purchased from Teknova (Hollister, CA, USA). Laemmli protein sample buffer, 4-15% 12-well precast polyacrylamide gel, Tris/glycine/SDS running buffer, Mini-PROTEAN™ Tetra Cell Electrophoresis System, and Bio-Safe™ Coomassie Stain were purchased from BioRad Laboratories (Hercules, CA, USA) Caco-2 human colorectal adenocarcinoma cells were bought from American Type Culture Collection (Manassas, VA, USA) while Dulbecco modified eagle medium (DMEM) with or without phenol red, fetal bovine serum (FBS), penicillin/streptomycin (P/S) solution, Hank's balanced salt solution (HBSS), Dulbecco's phosphate buffered saline (DPBS) and 0.25% trypsin solution were purchased from Thermo Fisher Scientific (Waltham, MA, USA). Intestinal epithelium growth medium comprising basal seeding medium (BSM), enterocyte differentiation medium (EDM) and MITO+ serum extender was purchased from Corning (Corning, NY, USA). Millicell®-PCF cell culture inserts (3.0 m pore size, 12 mm diameter) and TEER measuring device, Millicell®-ERS were obtained from Millipore Sigma (Burlington, MA, USA) while TEER measuring electrodes were obtained from World Precision Instruments, Inc (Sarasota, FL, USA). Paraformaldehyde (16% w/v) was purchased from Alfa Aesar (Ward Hill, MA, USA). Vectashield Hardset™ with 4′,6-diamidino-2-phenylindole, dihydrochloride (DAPI) was obtained from Vector laboratories Inc. (Burlingame, CA, USA). Porcine small intestine was obtained from CBSET Inc. (Lexington, MA, USA). Male Wistar rats weighing between 275-300 g were purchased from Charles River Laboratories (Wilmington, MA, USA). BD Lithium heparin-coated tubes were purchased from Becton, Dickinson and Company (Franklin Lanes, NJ, USA) Lucifer yellow was purchased from VWR (Radnor, PA, USA). All other reagents used were of analytical grade.

Preparation of CGLY variants and Antibody-CGLY preparation. CGLY variants were synthesized as previously reported^{[27]}. Briefly, glycolic acid dissolved in the minimum amount of ultrapure water needed for dissolution was reacted with choline bicarbonate (80 wt % solution) in a 2:1, 1:2, and 1:2 molar ratio (choline:glycolic acid) with constant stirring at 40° C. for 12 h until CO₂ evolution ceased. The residual water was removed by rotary evaporation at 20 mbar, 60° C. for 2 h followed by drying in a vacuum oven for 48 h at 60° C. Each CGLY formulation was characterized via Nuclear Magnetic Resonance (NMR) spectroscopy.

CGLY_{2:1}-¹H NMR (600 MHz, D₂O) 3.10 (s, 18H, NCH₃); 3.39 (n, 4H, NCH₂CH₂OH); 3.66 (d, 2H, HOCH₂OO); 3.82 (m, 4H, NCH₂CH₂OH)

CGLY_{1:1}-¹H NMR (600 MHz, DMSO) 3.10 (s, 9H, NCH₃); 3.39 (n, 2H, NCH₂CH₂OH); 3.66 (d, 2H, HOCH₂OO); 3.82 (m, 2H, NCH₂CH₂OH)

CGLY_{1:1}-¹H NMR (600 MHz, DMSO) 3.10 (s, 9H, NCH₃); 3.39 (m, 2H, NCH₂CH₂OH); 3.66 (d, 4H, HOCH₂OO); 3.82 (m, 2H, NCH₂CH₂OH)

IgG-CGLY formulations was prepared by adding predetermined amount of antibody to specific volume of CGLY, followed by gentle mixing for 1 min.

Physicochemical evaluation of antibody in CGLY variants by ELISA, circular dichroism and SDS PAGE: For the evaluation of antibody stability in CGLY variants, antibody-CGLY samples with 0.1 mg mL⁻¹ anti-human TNF-α IgG antibody concentration, with or without CGLY_{2:1}, CGLY_{1:1} and CGLY_{1:2} (20-90% v/v) were incubated for 1 h at room temperature (25° C.) and then dialyzed in 10 mM pH 7.4 sodium phosphate buffer (Boston BioProducts). After 48 h, the antibody samples were collected and assessed by enzyme-linked immunosorbent assay (ELISA). The TNFα-specific binding capability of dialyzed anti-human TNF-α IgG antibody-CGLY samples was assayed by ELISA. A 96-well ELISA plate was first coated overnight with 2 μg mL⁻¹ human TNFα using an ELISA coating buffer (Polysciences, Inc.). The wells were then blocked with Superblock™ Blocking Buffer (ThermoFisher Scientific) for 30 min before adding serially diluted dialyzed anti-human TNF-α IgG antibody samples as the primary antibody. After 2 h incubation, the wells were washed thrice with PBS containing 0.05% Tween 20 (PBST). HRP-conjugated goat anti-mouse IgG (Biolegend) was then used as the secondary antibody. The plate was incubated for 1 h before washing with PBST for 5 times. The ELISA plate was developed with a TMB substrate (Biolegend), and absorbance was measured at 450 nm with a Spectramax i3™ plate reader.

To analyze antibody stability using circular dichroism (CD) and SDS-PAGE, antibody-CGLY samples with 0.5 mg mL⁻¹ anti-human TNF-α IgG antibody concentration, with or without 50% v/v of CGLY_{2:1}, CGLY_{1:1} and CGLY_{1:2} were incubated for 1 h at room temperature (25° C.) and then dialyzed in 10 mM pH 7.4 sodium phosphate buffer (Boston BioProducts) for 48 h. Prior to CD measurement, the antibody concentrations were adjusted to 0.2 mg mL⁻¹. 400 μL of antibody samples were loaded in rectangular quartz cells (1-mm path length, Starna Cells, 1-Q-1) and CD spectra in the far-UV region (190-250 nm) indicating protein secondary structures were collected using CD spectrophotometry (Jasco J-1500). An SDS-PAGE assay was carried out to assess antibody aggregation of the antibody-CGLY samples. Specifically, all samples were adjusted to equivalent antibody concentrations in Laemmli protein sample buffer. The samples were then separated on a 4-15% 12-well precast polyacrylamide gel in Tris/glycine/SDS running buffer using a Mini-PROTEAN™ Tetra Cell Electrophoresis System (BioRad). The protein bands were stained with Bio-Safe™ Coomassie stain (BioRad) for observation according to manufacturer's protocol.

Caco-2 cell culture: Caco-2 cell line (human colorectal adenocarcinoma, ATCC HTB-37) was purchased from the American Type Culture Collection (ATCC) and maintained in Dulbecco's modified eagle medium (DMEM) supplemented with 10% fetal bovine serum (FBS) and 1% penicillin-streptomycin (100 U mL⁻¹ penicillium and 100 μg mL⁻¹ streptomycin) at 37° C. in a humidified atmosphere containing 5% CO₂.

Caco-2 cell viability evaluation of CGLY: Caco-2 cells suspended in supplemented DMEM were seeded at density of 150,000 cells mL⁻¹ and dispensed (100 μL per well) into 96-well plates. Each CGLY variants (CGLY_{2:1}, CGLY_{1:1} and CGLY_{1:2}) were diluted with supplemented DMEM to concentrations ranging from 1.875-480 mM. The media was aspirated from each well and each dilution was dispensed (100 μL per well) into 6 wells (6 cell replicates). Control wells were filled with media only. The cells were incubated with different concentrations of CGLY variants at 37° C., 5% CO₂ for 5 h followed by replacement of media with fresh DMEM (100 μL per well). The cells were allowed to grow for an additional 19 h (to a total of 24 h). Cell viability was assessed using the Cell Titer 96 AQueous™ One Solution cell proliferation assay (Promega Corporation), based on an MTS (3-(4,5-dimethylthiazol-2-yl)-5-(3-carboxymethoxyphenyl)-2-(4-sulfophenyl)-2H-tetrazolium) compound. In brief, 20 μL of the MTS reagent was added into each well, mixed gently, and incubated at 37° C. for 4 h. This was followed by reading the absorbance of the 96-well plate at 490 nm using Spectramax i3 plate reader. The conversion of MTS tetrazolium to formazan product as measured by absorbance at 490 nm was directly proportional to the number of living cells. The percentage of cell viability was calculated by subtracting the average absorbance of the no-cell control wells from all other experimental wells and assuming that the average absorbance from the wells containing nontreated cells represented 100% as suggested in the manufacturer's protocol.

Caco-2 monolayer culture transwells: For transport experiments in transwells, a 3-day rapid Caco-2 growth system was used. Cells were placed in Corning® basal seeding medium (BSM) supplemented with MITO serum+ extender and seeded at density of 400,000 cells mL⁻¹ on Millicell® PCF inserts placed inside 24-well plates. 500 μL of cells containing medium was placed in apical side while 1000 μL of cell free BSM was put in the basolateral side as per manufacturer recommendation. After 24 h of incubation at 37° C., 5% CO₂, the medium was replaced with same volume of enterocyte differentiation medium supplemented with MITO serum+ extender for another 2-4 days. TEER was measured on a regular basis and when it reached above 200 ohms·cm², indicating sufficient tight junction integrity between cells, transport study was performed.

FITC-IgG and lucifer yellow transport through Caco-2 monolayer transwells: Prior to the experiment, the Caco-2 transwells were washed twice with HBSS and then incubated with DMEM devoid of phenol red, FBS and P/S in both the apical (400 μL) and basolateral side (600 μL) for 30 minutes. Thereafter, the medium in the apical side was replaced with 400 μL of 500 μg mL⁻¹ of either FITC-IgG or Lucifer yellow prepared with 0, 30, 55 or 80 mM CGLY and solubilized in DMEM free of phenol red, FBS and P/S. Immediately after addition of FITC-IgG at the apical side, a 150 μL aliquot was withdrawn from the basolateral side and replaced with equal volume fresh DMEM. This was repeated at 1, 2, 3, 4 and 5 h. During the study, the transwell plates were placed inside an incubator at 37 QC, 5% CO₂ on a shaker rotating at 100 rpm and only taken out to remove aliquots at the aforementioned time periods. After the end of study at 5 h, the FITC-IgG and Lucifer yellow concentration in the aliquots were measured using BioTek, Synergy Neo2™ plate reader (Vermont, USA) at 485/520 nm and 485/530 nm excitation/emission wavelengths, respectively. The FITC-IgG and Lucifer yellow concentrations for each time point was calculated from calibration solutions of each fluorescent molecule, which was then plotted as the basolateral chamber concentration versus time.

For qualitative analysis of FITC-IgG uptake by Caco-2 cells, the transwells from FITC-IgG transport study were washed two times with HBSS at the end of study, followed by addition of 500 μL of 4% paraformaldehyde and kept at 4° C. overnight. On the next day, paraformaldehyde was aspirated from the wells, membranes washed with PBS two times and the transwell membrane were cut and gently placed on glass slides. Mounting media containing DAPI was added to the membranes and covered with cover slips. Confocal imaging of the membranes (ZEISS, laser scanning confocal microscope LSM 700) was taken at 40× magnification.

TEER measurement of CGLY_{2:1} treated Caco-2 monolayer transwells: Once the Caco-2 transwells were washed and incubated with DMEM devoid of phenol red, FBS and P/S for 30 minutes. TEER values were recorded for each insert. Thereafter, the medium in the apical side was replaced with 400 μL of 0, 30, 55 or 80 mM CGLY_{2:1}. During the study, the transwell plates were placed inside an incubator at 37° C., 5% CO₂ on a shaker rotating at 100 rpm and only taken out to perform additional TEER measurements at 1, 2, 3, 4, 5 and 24 h to determine TEER recovery and tight junctions reversibility. TEER was plotted as % change from initial value versus time.

FITC-IgG transport with transcytosis inhibitors: Prior to the experiment, the Caco-2 transwells were washed twice with HBSS and then incubated with DMEM devoid of phenol red, FBS and P/S in both the apical (400 μL) and basolateral side (600 μL) for 30 minutes. Thereafter, the medium in the apical side was replaced with 400 μL DMEM free of phenol red, FBS and P/S containing 500 μg mL⁻¹ FITC-IgG, 55 mM CGLY_{2:1}, with or without transcytosis inhibitors including 50 μM monodansylcadaverine (MDC), 1 μg mL⁻¹ filipin, and 0.5 μM wortmannin^{[36]}. After 24 h incubation, 150 μL aliquots were withdrawn from the basolateral side and FITC-IgG concentration in the aliquots were measured using BioTek, Synergy Neo2™ plate reader (Vermont, USA) at 485/520 nm excitation/emission wavelengths and plotted as the FITC-IgG transport percentage compared to control wells without any transcytosis inhibitors.

Mucus rheology studies: Porcine small intestinal mucus was extracted from porcine intestine by gently scraping the surface of the washed mucosa with a small laboratory spatula avoiding, as far as possible, the removal of epithelial cells^{[39]}. Porcine mucus was pooled and investigated immediately. 10 uL of 0, 12.5, 25 and 50% v/v of CGLY in 0.9% saline were added to 200 uL of porcine mucus aliquots and viscosity was measured across a shear rate range of 1-100 l/s at 25° C. using an AR-G2 rheometer with a 40 mm diameter steel parallel plate geometry (TA Instruments, New Castle, DE, USA).

In vivo local delivery of Antibody-CGLY via Intrajejunal Administration: All Experiments pertaining to the use of animals were performed in accordance to the protocols approved by the Institutional Animal Care and Use Committee of Harvard University. Prior to the study, 275-300 g adult male Wistar rats were fasted overnight but given free access to water. On the day of the experiment, the rats were anesthetized and injected with 200 μL of 1 mg mL⁻¹ FITC-labeled IgG antibody (FITC-IgG) in 50% v/v CGLY_{2:1} in saline or in saline (n=3). Rats receiving equivalent saline injection without FITC-IgG were used as the negative control group. After 2 h, the rats were sacrificed and jejunal tissues were harvested and preserved using Swiss-rolling technique^{[14]}. The rolled tissues were then fixed in 4% paraformaldehyde at 4° C. for 12 h, transferred to 4.5% sucrose at 4° C. for 4 h and finally to 20% sucrose at 4° C. for 12 h^{[41]}. The tissues were then frozen at −80° C. in the presence of optimum cutting temperature (OCT) compound and tissue sections were cut into 25 m thickness. was visualized using a slide scanner microscope (ZEISS Axio Scan.Z1) and the images processed using Zen™ (Blue edition) software.

In vivo systemic delivery of Antibody-CGLY via Intrajejunal Administration: The study was performed on adult male Wistar rats fasted overnight but given free access to water. Before the start of the study, the rats were anesthetized, abdominal hair clipped, and the surgery area was prepped using betadine and 70% ethanol. An incision was made in the abdomen to expose the intestine and test formulations were injected in the jejunum. The time zero blood was taken after the intestine was exposed i.e. immediately prior to the injections. Each group of 6 rats was injected with 200 μg kg⁻¹ of 0.3 mg mL⁻¹ anti-human TNF-α IgG antibody in 50% v/v CGLY_{2:1} in saline or in saline alone. Thereafter, intestinal section was placed back into the abdomen and the muscle and skin sutured. Loss in body temperature in the animals during anesthesia was prevented by placing the animals on temperature controlled warming pads prior to surgery followed by additional towel cover after surgery. The animals remained anesthetized throughout the study and were euthanized after 5 h. The anti-human TNF-α IgG concentration in blood plasma was evaluated by collecting around 250 μL blood in heparinized-coated tubes at 0, 0.5, 1, 1.5, 2, 3 and 5 h from treated rats. Standard protocol was followed to isolate plasma from whole blood. Blood samples were centrifuged at 2,000×g for 15 mins. The plasma supernatant was immediately transferred into clean tubes, stored in ice during the procedure and subsequently at −20 □ C till further analysis of IgG content. The evaluation of anti-human TNF-α IgG concentration in the plasma samples at each time point was performed by ELISA as previously described and calculated from calibration solutions of anti-human TNF-α IgG.

In vivo toxicity studies: To evaluate the acute toxicity of the CGLY_{2:1} in vivo, adult male Wistar rats (n=6, 275-300 g each) were orally administered with 50% v/v CGLY_{2:1} in saline at a dose of 625 mg/kg once daily for 7 consecutive days using the procedure as described above. Control rats were administered with equivalent saline dosage. During the experimental period, the rat body weight was monitored daily. On day 7, rats were sacrificed, blood samples were collected for comprehensive metabolic panel analysis, and major organs and gastrointestinal tissues were processed for histological examination. Heart, liver, spleen, lung, kidney, and gastrointestinal (stomach, small intestine and colon) tissues were fixed in neutral-buffered 10 v/v % formalin for 18 h, dehydrated in 70% ethanol, and then embedded in paraffin. The tissue sections were cut into 5 m thickness, deparaffinized, rehydrated, and stained with hematoxylin and eosin (H&E). Histological morphology was visualized using a brightfield slide scanner microscope (ZEISS Axio Scan.Z1™) and the images processed using Zen™ (Blue edition) software.

Statistical Analysis: All data are presented as mean±SE. For SDS PAGE studies, the experiments were performed in triplicate and a representative image was shown. In fluorescence and brightfield imaging, experiments were performed in triplicate and a representative image was shown. All other experiments were conducted in at least triplicates. To examine the statistical significance, unpaired two-tailed t-tests were performed in GraphPad Prism 8™ with confidence level P=0.05 deemed significant.

### REFERENCES

[1] S. Awwad, U. Angkawinitwong, Pharmaceutics 2018, 10.
[2] R. J. Keizer, A. D. Huitema, J. H. Schellens, J. H. Beijnen, Clin. Pharmacokinet. 2010, 49, 493.
[3] A. C. Anselmo, Y. Gokam, S. Mitragotri, Nat. Rev. Drug Discov. 2019, 18, 19.
[4] J. T. Ryman, B. Meibohm, CPT Pharmacometrics Syst. Pharmacol. 2017, 6, 576.
[5] S. J. Green, J. Brendsel, Gut 2006, 55, 1681.
[6] A. Matucci, A. Vultaggio, R. Danesi, Respir. Res. 2018, 19, 154.
[7] D. E. Johnson, Int. J Mol. Sci. 2018, 19.
[8] R. G. Jones, A. Martino, Crit. Rev. Biotechnol. 2016, 36, 506.
[9] A. Bak, M. Ashford, D. J. Brayden, Adv. Drug Deliv. Rev. 2018,136-137, 2.
[10] E. Moroz, S. Matoori, J. C. Leroux, Adv. Drug Deliv. Rev. 2016, 101, 108.
[11] J. O. Morales, K. R. Fathe, A. Brunaugh, S. Ferrati, S. Li, M. Montenegro-Nicolini, Z. Mousavikhamene, J. T. McConville, M. R. Prausnitz, H. D. C. Smyth, AAPSJ 2017, 19, 652.
[12] M. Vancamelbeke, S. Vermeire, Expert Rev. Gastroenterol. Hepatol. 2017, 11, 821.
[13] M. S. Harris, D. Hartman, B. R. Lemos, E. C. Erlich, S. Spence, S. Kennedy, T. Ptak, R. Pruitt, S. Vermeire, B. S. Fox, J Crohns Colitis 2016, 10, 631.
[14] J. S. Crowe, K. J. Roberts, T. M. Carlton, L. Maggiore, M. F. Cubitt, S. Clare, K. Harcourt, J. Reckless, T. T. MacDonald, K. P. Ray, A. Vossenkamper, M. R. West, Sci. Rep. 2018, 8, 4941.
[15] W. R. Strohl, Protein Cell 2018, 9, 86.
[16] A. Muheem, F. Shakeel, M. A. Jahangir, M. Anwar, N. Mallick, G. K. Jain, M. H. Warsi, F. J. Ahmad, Saudi Pharm. J. 2016, 24, 413.
[17] D. Vllasaliu, M. Thanou, S. Stolnik, R. Fowler, Expert Opin. Drug Deliv. 2018, 15, 759.
[18] B. Esteban-Fernindez de Avila, P. Angsantikul, J. Li, W. Gao, L. Zhang, J. Wang, Adv. Funct. Mater. 2018, 28, 1705640.
[19] J. Li, P. Angsantikul, W. Liu, B. Esteban-Fernandez de Avila, S. Thamphiwatana, M. Xu, E. Sandraz, X. Wang, J. Delezuk, W. Gao, L. Zhang, J. Wang, Angew. Chem. Int. Ed. Engl. 2017, 56, 2156.
[20] B. E. de Avila, P. Angsantikul, J. Li, M. Angel Lopez-Ramirez, D. E. Ramirez-Herrera, S. Thamphiwatana, C. Chen, J. Delezuk, R. Samakapiruk, V. Ramez, M. Obonyo, L. Zhang, J. Wang, Nat. Commun. 2017, 8, 272.
[21] X. Wei, M. Beltran-Gastelum, E. Karshalev, B. Esteban-Fernandez de Avila, J. Zhou, D. Ran, P. Angsantikul, R. H. Fang, J. Wang, L. Zhang, Nano Lett. 2019, 19, 1914.
[22] B. R. Carrillo-Conde, E. Brewer, A. Lowman, N. A. Peppas, Ind. Eng. Chem. Res. 2015, 54, 10197.
[23] X. Y. Chen, A. M. Butt, M. C. I. Mohd Amin, J. Control. Release 2019, 311-312, 50.
[24] S. H. Lee, J. G. Song, H. K. Han, J. Control. Release 2019, 311-312, 74.
[25] W. Fan, D. Xia, Q. Zhu, X. Li, S. He, C. Zhu, S. Guo, L. Hovgaard, M. Yang, Y. Gan, Biomaterials 2018, 151, 13.
[26] C. Agatemor, K. N. Ibsen, E. E. L. Tanner, S. Mitragotri, Bioeng. Transl. Med. 2018, 3, 7.
[27] E. E. L. Tanner, A. M. Curreri, J. P. R. Balkaran, N. C. Selig-Wober, A. B. Yang, C. Kendig, M. P. Fluhr, N. Kim, S. Mitragotri, Adv. Mater. 2019, 31, e1901103.
[28] I. M. Marrucho, L. C. Branco, L. P. Rebelo, Annu. Rev. Chem. Biomol. Eng. 2014, 5, 527.
[29] M. Zakrewsky, A. Banerjee, S. Apte, T. L. Kern, M. R. Jones, R. E. Sesto, A. T. Koppisch, D. T. Fox, S. Mitragotri, Adv. Healthc. Mater. 2016, 5, 1282.
[30] V. Joshi, T. Shivach, N. Yadav, A. S. Rathore, Anal. Chem. 2014, 86, 11606.
[31] A. W. Vermeer, M. G. Bremer, W. Norde, Biochim. Biophys. Acta 1998, 1425, 1.
[32] C. S. Cheung, K. W. Anderson, P. M. Patel, K. L. Cade, K. W. Phinney, I. V. Turko, Sci. Rep. 2017, 7, 42497.
[33] R. R. Mazid, R. Vijayaraghavan, D. R. MacFarlane, C. Cortez-Jugo, W. Cheng, Chem. Commun. (Camb.) 2015, 51, 8089.
[34] R. Konsoula, F. A. Barile, Toxicol. In Vitro 2005, 19, 675.
[35] V. Gupta, N. Doshi, S. Mitragotri, PLoS One 2013, 8, e57136.
[36] R. Ghaffarian, S. Muro, J. Vis. Exp. 2013, e50638.
[37] A. R. Mackie, A. Macierzanka, K. Aarak, N. M. Rigby, R. Parker, G. A. Channell, S. E. Harding, B. H. Bajka, Food Hydrocoll. 2016, 52, 749.
[38] M. Boegh, S. G. Baldursdottir, A. Mullertz, H. M. Nielsen, Eur. J. Pharm. Biopharm. 2014, 87, 227.
[39] L. A. Sellers, A. Allen, E. R. Moms, S. B. Ross-Murphy, Biochim Biophys Acta 1991, 1115, 174.
[40] A. B. Bialkowska, A. M. Ghaleb, M. O. Nandan, V. W. Yang, J. Vis. Exp. 2016.
[41] H. Ochi, M. Abraham, H. Ishikawa, D. Frenkel, K. Yang, A. S. Basso, H. Wu, M. L. Chen, R. Gandhi, A. Miller, R. Maron, H. L. Weiner, Nat. Med. 2006, 12, 627.

### Example 2

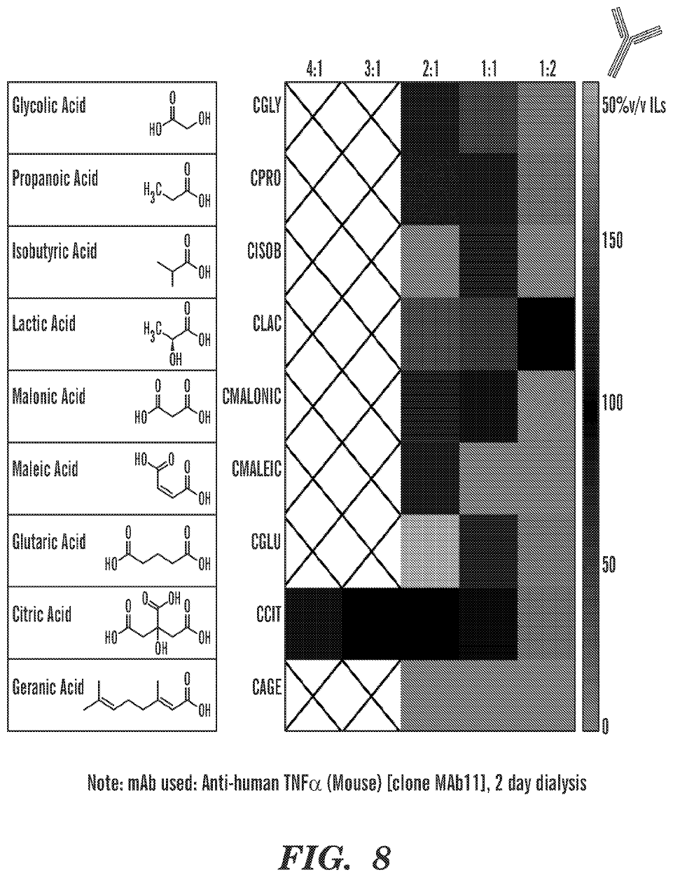

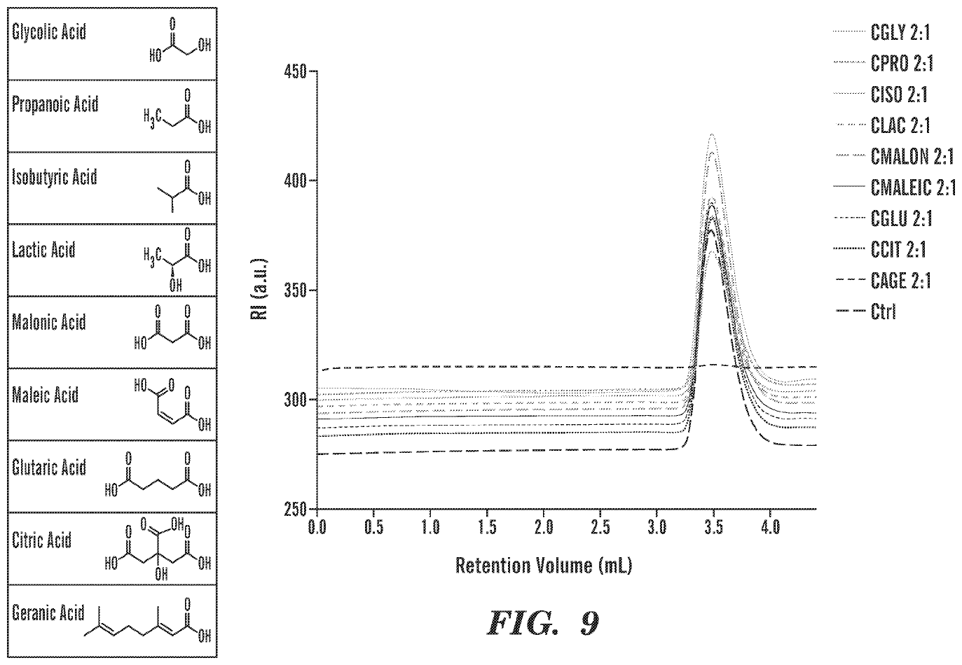

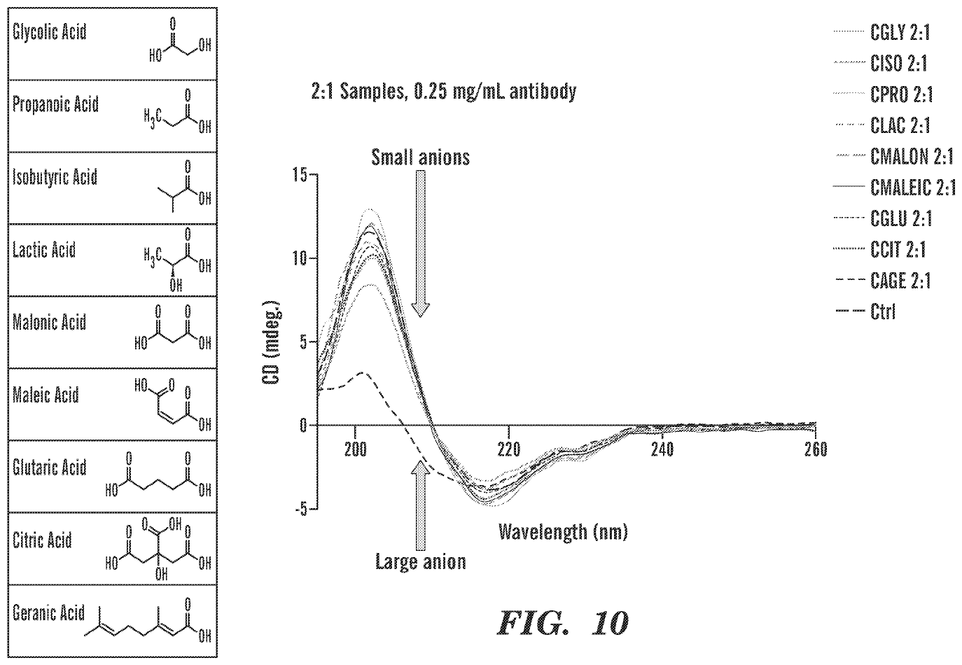

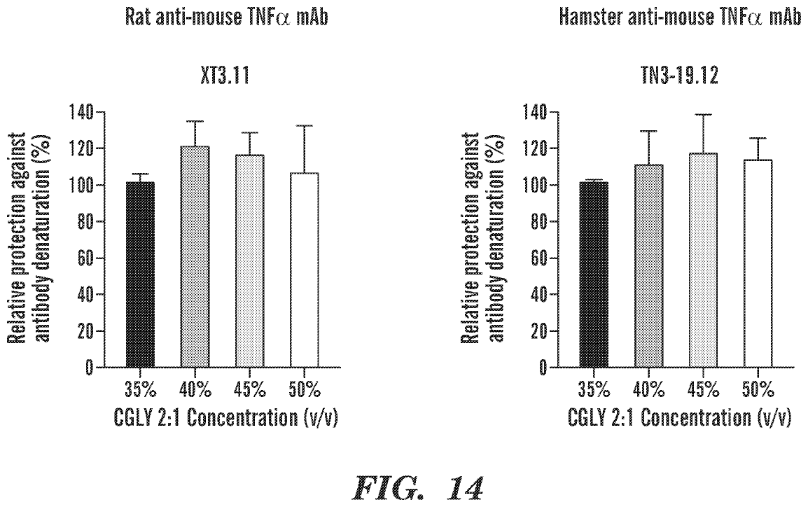

Multiple ILs were tested to determine how well they promoted functional antibody stability (FIGS. 8-10). As a general trend, small anions are more compatible with antibodies than larger anions. Performance was retained with different individual antibodies (FIG. 14).

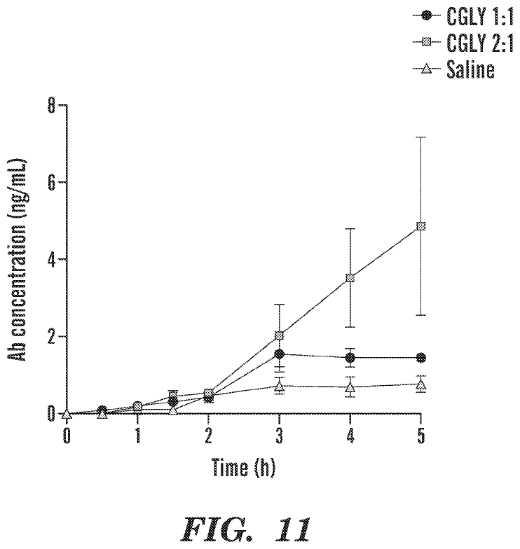

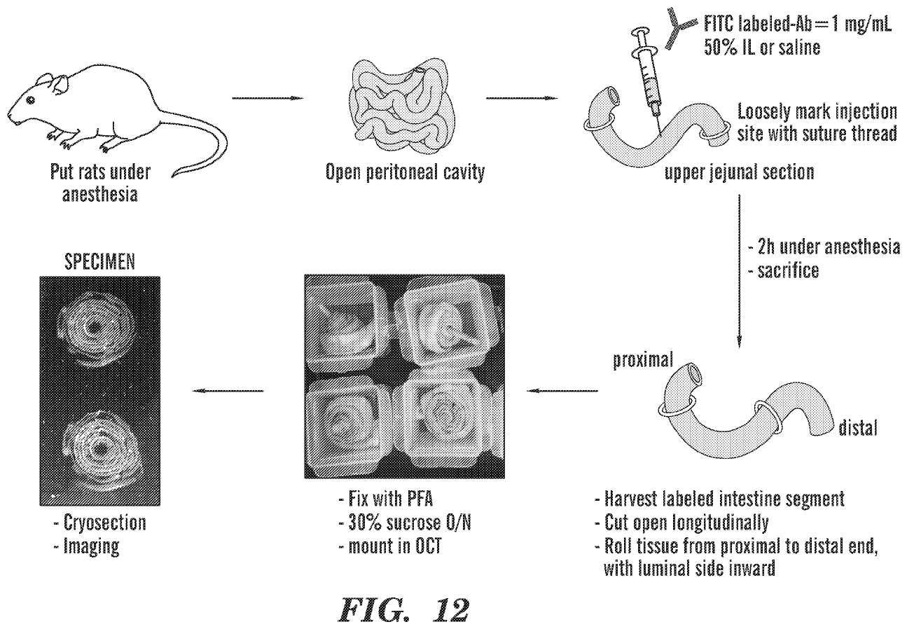

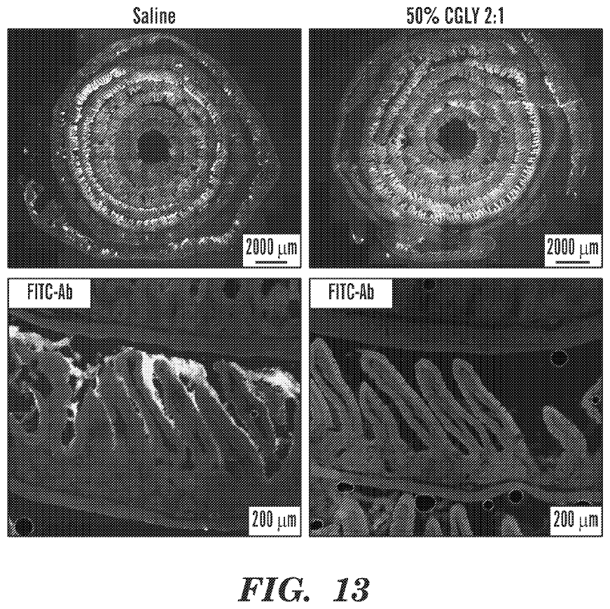

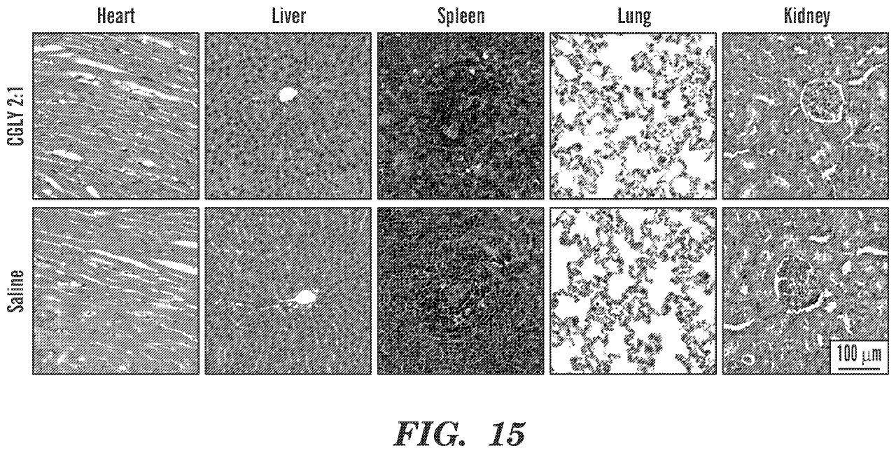

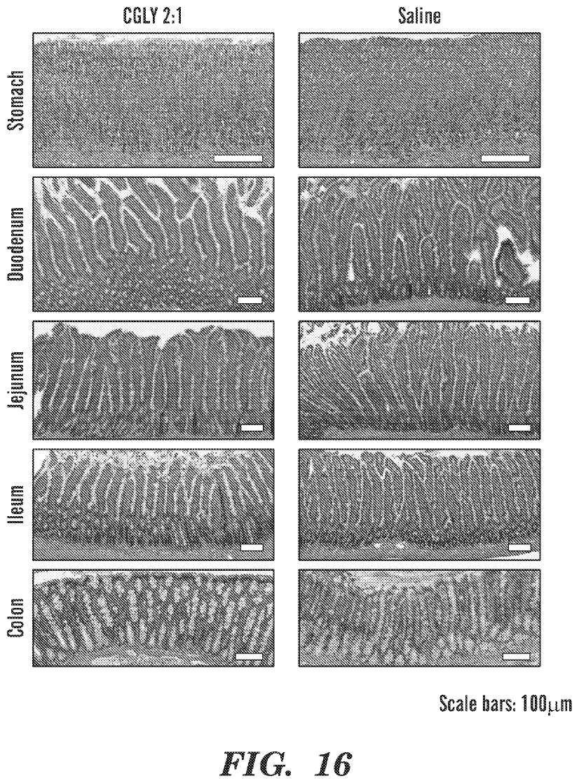

Antibody delivery, orally or by intrajejuenal administration was tested as well (FIGS. 11-13). The ILs were confirmed not to be toxic when administered orally (FIGS. 15-16).

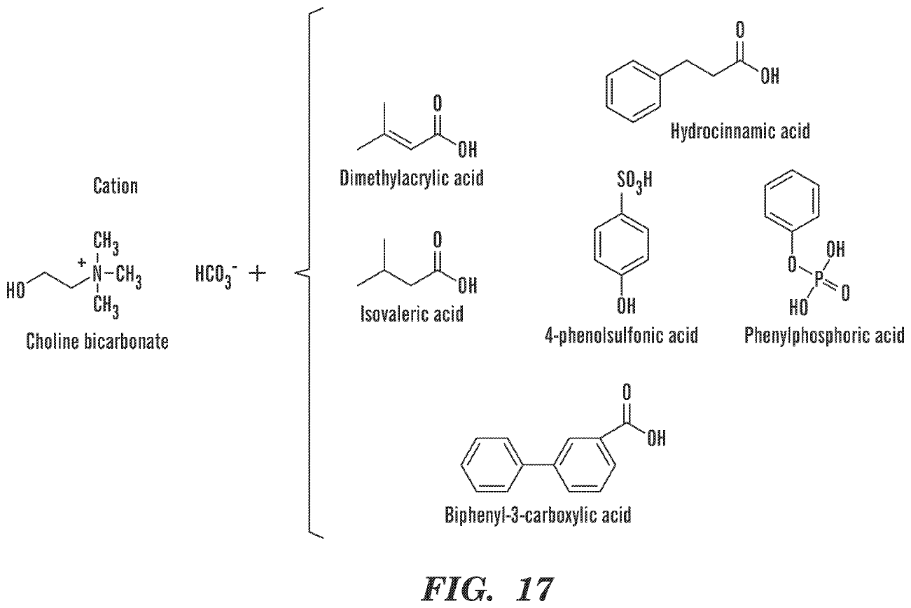

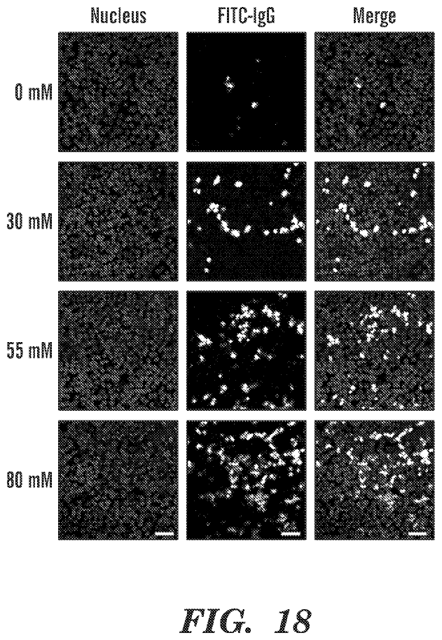

ILs for siRNA delivery are also contemplated and transdermal of siRNA was tested (FIGS. 17-18).

### Example 3

Systemic antibodies targeting tumor necrosis factor-α (TNF-α) and interleukin-17A (IL-17A) are effective in plaque psoriasis. Despite their popularity, safety concerns pose a challenge for systemic biologics. While anti-TNF-α and anti-IL-17A antibodies effectively inhibit respective proteins, the inventors hypothesized that an approach based on local silencing of an upstream target such as NFKBIZ would be advantageous for treating psoriasis. However, effective delivery of small interfering RNA (siRNA) into the skin is a substantial hurdle due to skin's barrier function and poor stability of siRNA. Using ionic liquids as an enabling technology, described herein is the effective delivery of NFKBIZ siRNA into the skin and its therapeutic efficacy in a psoriasis model. Treatment with IL-siRNA suppressed aberrant gene expression and resulted in down-regulation of psoriasis-related signals including TNF-α and IL-17A. These results provide a framework for a topical delivery platform for siRNA.

### Introduction

Psoriasis is one of the most debilitating chronic skin diseases affecting more than 125 million people worldwide with an estimated economic burden of $135 billion/year in the United States (1). Its pathogenesis and the underlying mechanisms are still not fully understood. Nuclear factor KB (NF-κB), a ubiquitously expressed transcription factor, is considered as the master regulator of immune responses and is implicated in several autoimmune inflammatory diseases including psoriasis (2). Several therapeutics targeting NF-κB signaling pathways are available in the clinic; however, concerns regarding the lack of specificity and side effects pose a challenge (3). This is particularly challenging since systemic inhibition of pleotropic proteins like NF-κB might lead to serious side effects as they provide essential basal activity as survival factors. Network-centric approaches involving pathway-specific inhibitors have gained considerable therapeutic interests (4). In this regard, infliximab and adalimumab [both anti-tumor necrosis factor-α (TNF-α) monoclonal antibodies] as well as secukinumab [an anti-interleukin-17A (IL-17A) antibody] have been approved by the U.S. Food and Drug Administration and are claimed to mediate their therapeutic effects through the modulation of NF-κB activity (5).

NFKBIZ, a gene encoding atypical inhibitor of nuclear factor KB (IκB) protein IκBζ, has gained interests for therapeutic intervention due to its crucial role in the regulation of NF-κB complexes (6, 7). It is reported to be a direct transcription activator of TNF-α-, IL-17A-, and IL-36-inducible psoriasis-related gene products that are involved in inflammatory signaling, neutrophil chemotaxis, and leukocyte activation (8-11). In addition, strong expression of NFKBIZ in patients with psoriasis could be correlated to elevated IL-36- and IL-17A-type responses (12). Local silencing of NFKBIZ can be advantageous since it can potentially broaden the population of patients that can benefit from the treatment compared with that by a single antibody.

Silencing of NFKBIZ through topical applications of small interfering RNA (siRNA) offers a noninvasive and self-administered treatment option with minimal side effects (13). However, the greatest challenge of this route is that only a limited number of drugs with low molecular weights (up to few hundred daltons) and high octanol-water partition coefficients are usable for successful topical delivery (14). Transdermal and topical delivery of hydrophilic molecules, particularly macromolecules such as antibodies and nucleic acids, remains challenging, owing to their high molecular weights (15). Several reports have showcased topical siRNA delivery using techniques such as spherical nucleic acids (16) and self-assembling framework nucleic acids (17). Microneedles have also been explored for topical delivery of siRNA (18). Methods such as electroporation (19) and peptide carriers have also been explored (20-22). Strategies have also been developed to deliver siRNA to treat cutaneous wounds (23, 24).

Described herein is a modular IL-based siRNA delivery approach for silencing various genes of interest. Specifically, described herein is a combination of ILs that simultaneously stabilizes siRNA and enhances siRNA penetration into the skin following topical application. The efficacy of the formulation in silencing NFKBIZ in vivo in an imiquimod-induced psoriasis mouse model is demonstrated.

Results

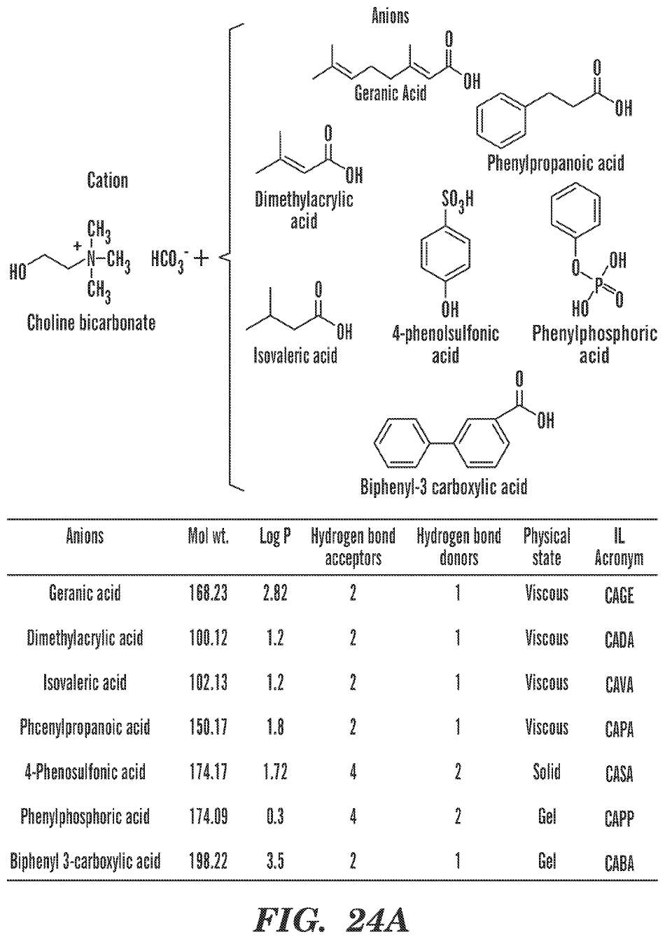

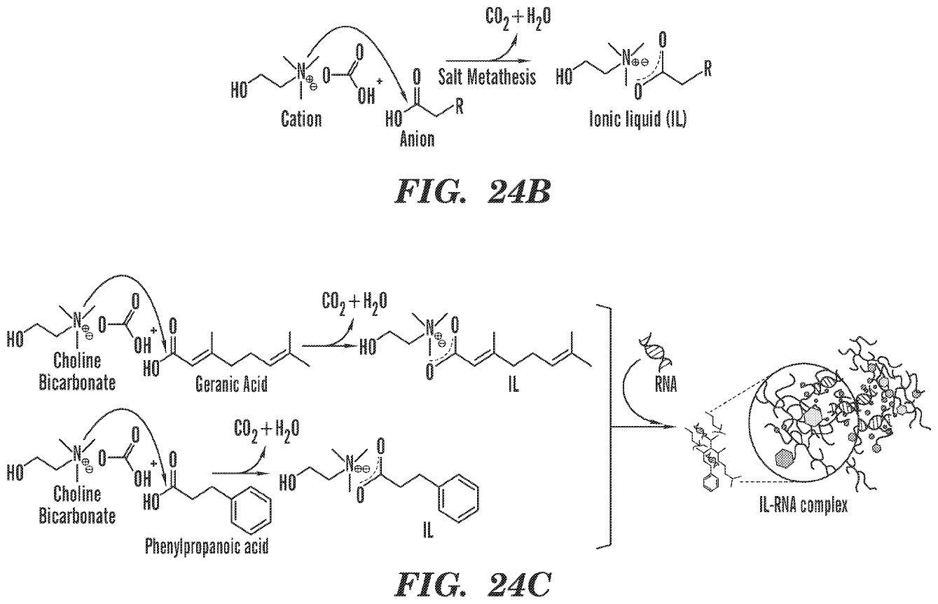

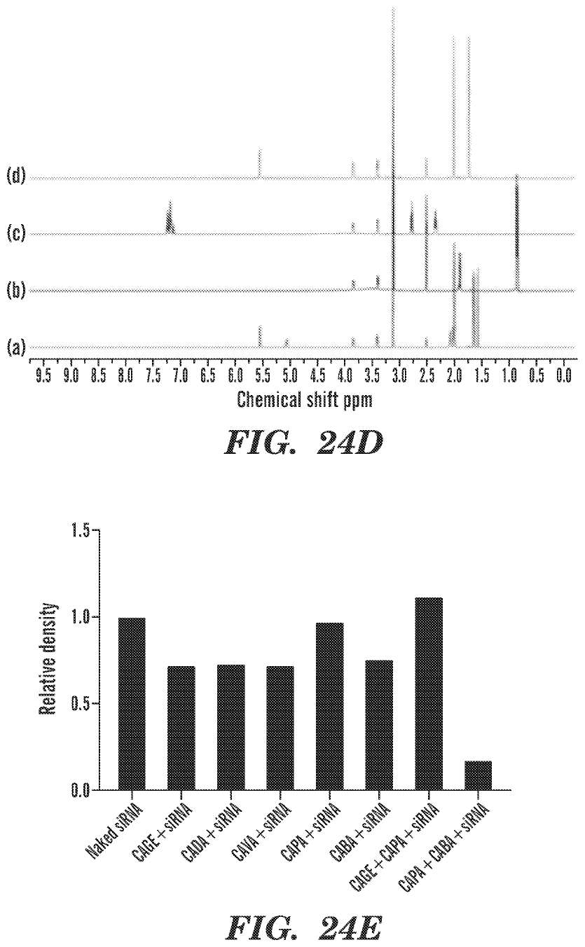

IL selection. A library of ILs was designed and synthesized to assess siRNA delivery into skin. Cholinium was used as the cation in all ILs due to its biocompatibility. Several different anions were used to synthesize ILs (FIGS. 24A-24E). Geranic acid was used as the reference anion in the IL library [that is, choline and geranic acid (CAGE) as a reference IL]. Other anions were chosen for several reasons. First, anions containing shorter linear carbon chains were chosen in contrast to geranic acid to assess the impact of the chain length on siRNA stability and delivery. Anions with aromatic groups were chosen since they might interact with the stacked RNA base pairs via electrostatic, hydrophobic, and polar interactions. All ILs were prepared at a stoichiometric ratio of 1:2 (cation:anion) and were assessed for stability and siRNA delivery. Of the ILs synthesized, CAGE, choline and dimethylacrylic acid (CADA), choline and isovaleric acid (CAVA), and choline and phenylpropanoic acid (CAPA) remained as a viscous liquid at room temperature (RT), whereas choline and 4-phenolsulfonic acid (CASA), choline and phenylphosphoric acid (CAPP), and choline and biphenyl-3-carboxylic acid (CABA) solidified or formed a gel (FIGS. 24A-24E). Representative 1H nuclear magnetic resonance (NMR) spectra can be found in FIGS. 24A-24E, confirming the successful synthesis and purity of the ILs. In addition, since both interleukin and ILs have been denoted as “IL,” for the purpose of clarity, all interleukins are referred by a numerical value throughout the manuscript.

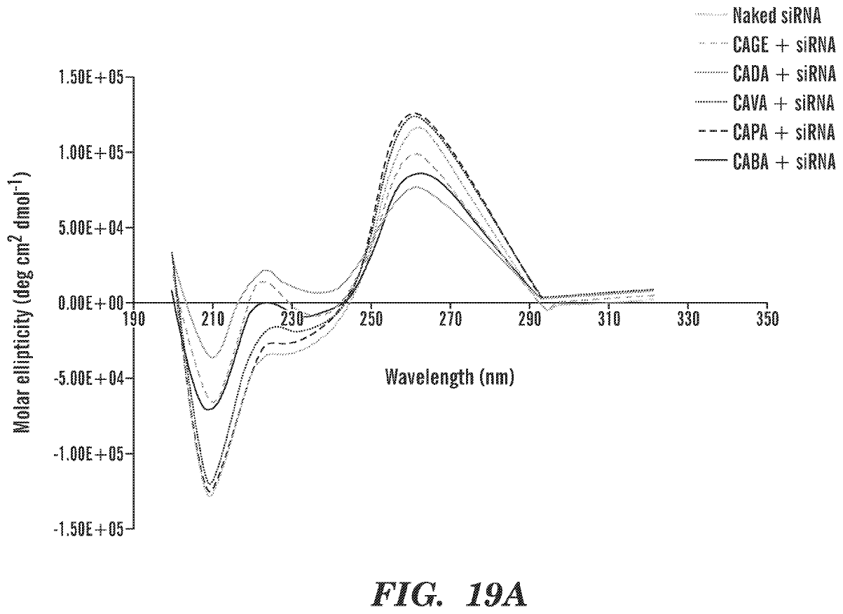

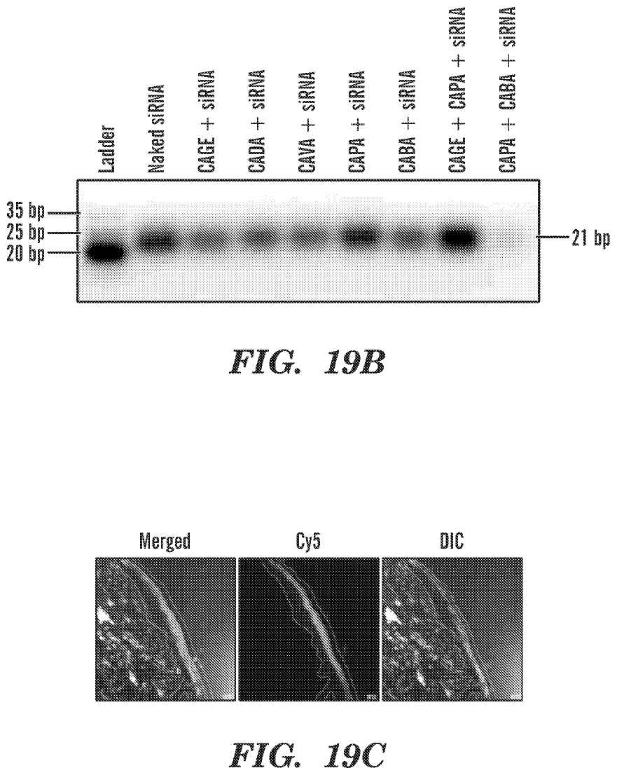

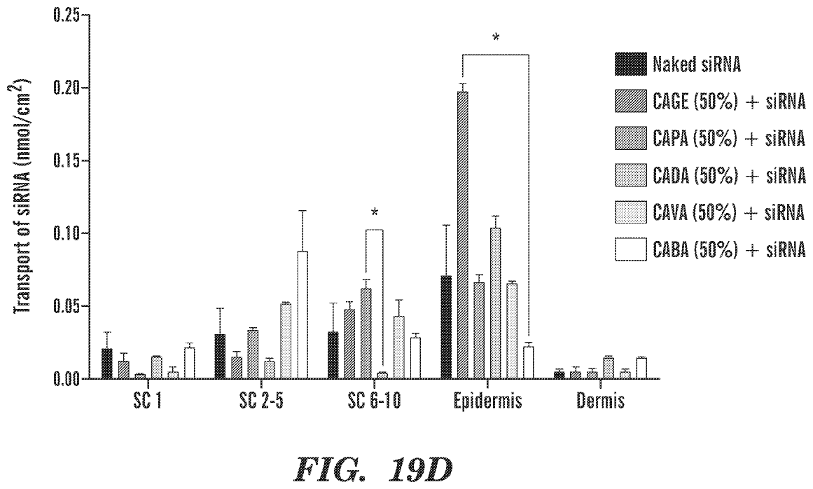

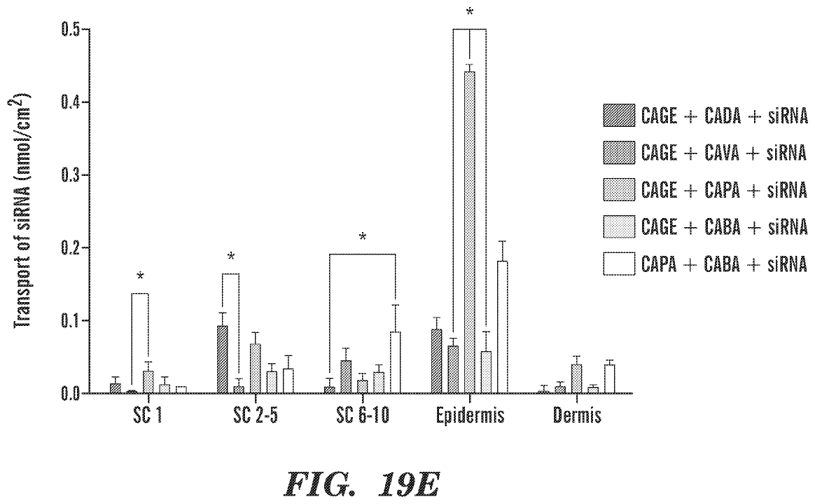

Effect of ILs on siRNA stability. The effect of ILs on siRNA stability was assessed. Circular dichroism (CD) spectroscopy of siRNA incubated with aqueous solutions of individual ILs at 50% (v/v) concentration revealed notable alteration in the a helix backbone (confirmed from the negative band at 210 nm) in the presence of CAGE, CADA, and CABA. On the other hand, CAVA and CAPA retained the secondary structure of siRNA (FIG. 19A). Bands obtained from the native gel electrophoresis complemented with the CD results (FIG. 19B). The improved stability of siRNA in the presence of CAPA suggested the possibility of synergistic effects between the ILs prepared from two structurally different anions. Consequently, the effect of IL mixtures on siRNA stability was assessed to determine whether the compatibility of CAPA with siRNA might offer additional protection against the adverse effects of CAGE and CABA on the siRNA structure. The combination of CAGE (25% v/v) and CAPA (25% v/v) led to a prominent band indicative of retention of siRNA structure (FIGS. 24A-24E).

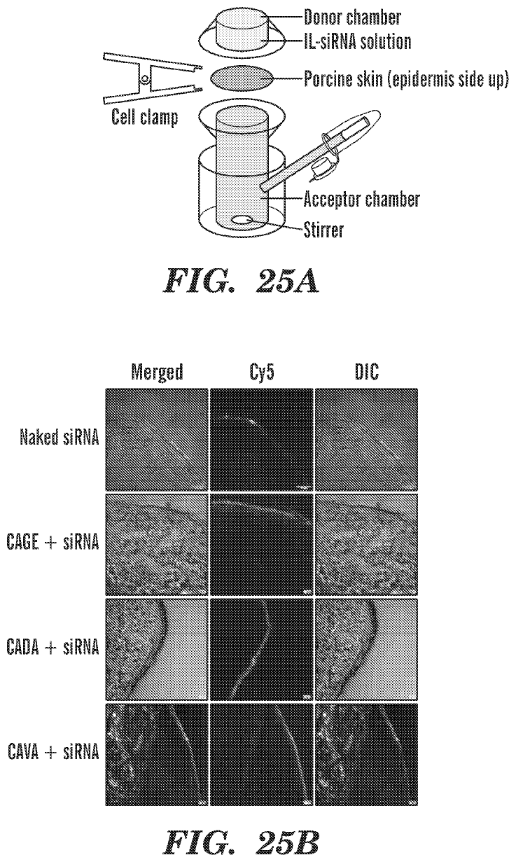

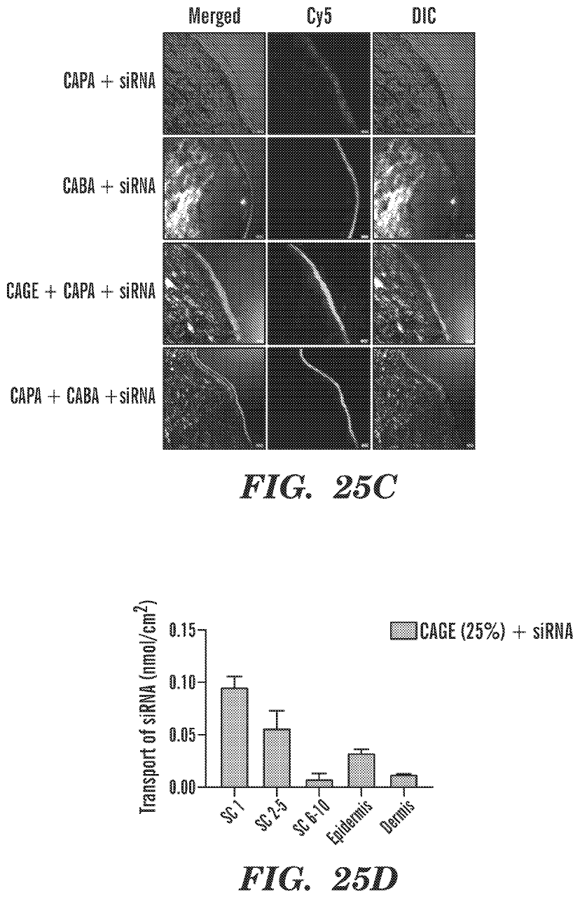

Screening of optimal IL combinations for siRNA delivery. The individual ILs and their combinations were then evaluated for epidermal permeation of Cy5-labeled siRNA into porcine skin in Franz diffusion cells (FDCs) (FIG. 19C). Some epidermal uptake for naked siRNA was seen in controls. CAGE exhibited the highest delivery among all tested ILs (FIG. 19D). About 0.20 nmol/cm2 of siRNA was delivered into the epidermis in the presence of CAGE (50% v/v) compared with 0.07 nmol/cm2 in case of naked siRNA. Since 50% CAGE had a potential effect on the siRNA structure, the ability of IL combinations to deliver siRNA into skin was also measured. A combination of CAPA and CAGE (25% v/v each) led to ˜0.4 nmol/cm2 siRNA getting delivered into the skin (FIG. 19E). Because the CAGE+CAPA combination yielded the highest epidermal delivery as well as high stability, it was selected as the lead formulation for further studies (FIGS. 25A-25D).

IL-induced intercalation and solvating effects on RNA. Molecular dynamic (MD) simulations were performed to explore the mechanism by which the IL combination (CAGE+CAPA) stabilizes the RNA. It is evident from the snapshots of unit cells within 10 Å of RNA that geranic acid in CAGE is responsible for forming aggregated clumps, leading to separation of geranic acid from choline, water, and the RNA molecule (FIGS. 20A-20B). Addition of phenylpropanoic acid to CAGE led to a more consistent distribution of the three molecular species/ions in the IL solution (FIGS. 20C-20D). Furthermore, the proximity of phenylpropanoic acid molecules to the RNA molecules, possibly due to the presence of hydrophobic aromatic rings unlike its aliphatic counterpart (geranic acid), confirms its crucial role in intercalating between the stacked RNA base pairs contributing to the RNA solvation and stability.

Structural properties of RNA were assessed by performing simulations over the course of 500 ns and measuring the root mean square deviation (RMSD) and radius of gyration (RGYR). The RGYR obtained for the CAGE group was consistent up to 150 ns and started decreasing toward the end of the simulation, indicating the inconsistent compactness of the system (FIG. 20E). In contrast, the increased and consistent RGYR obtained for the IL combination (CAGE+CAPA) over 500 ns aligns well with the improved IL-RNA interaction results. Such improved interactions and compactness for the optimized IL system with the RNA could also be attributed to the increase in the relative molecular mobility or reduced local viscosity upon addition of phenylpropanoic acid to CAGE. In addition, lower viscosity of the IL system may weaken the intramolecular strain placed on the RNA by the IL and is a possible explanation for the reduced RMSD observed in the case of CAGE+CAPA (FIG. 20F).

IL-mediated lipid membrane dynamics modulation. To assess the insertion and translocation of the IL into the lipid bilayers, simulations of the lipid bilayer in the presence of IL were conducted (FIGS. 21A-21C). In addition to improving the stability and solvation of the RNA, the compact packing of the ionic species leading to the formation of aggregates seems to augment the IL-lipid membrane interactions. The aggregates formed by the individual ionic moieties appear to enable a continuity between the IL system and the molecules, making up the lipid bilayer. It is possible that the collective mass of the ionic aggregates plays a crucial role in facilitating membrane permeation in addition to ILs, particularly geranic acid's ability to extract or fluidize lipids as previously reported (26).

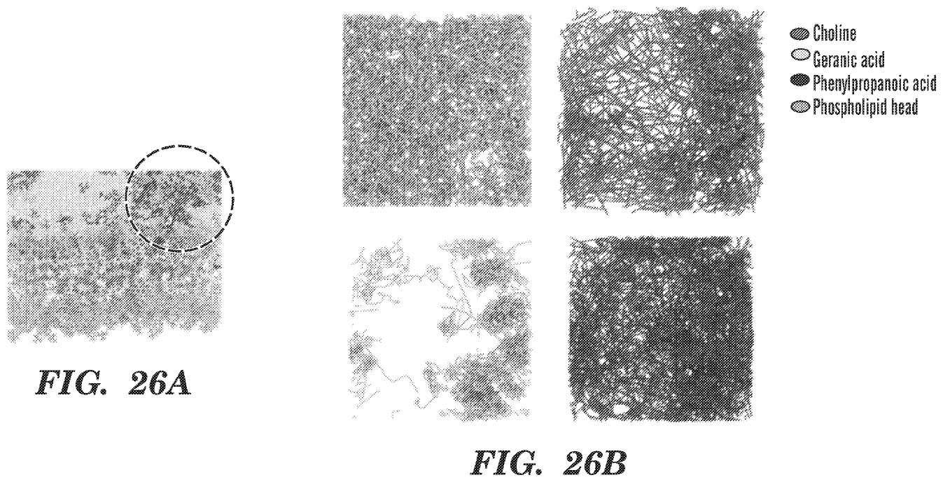

The relative effect of the ILs including CAGE, CAPA, and CAGE+CAPA on membrane dynamics was assessed by measuring the average thickness of the lipid bilayer in the presence of ILs over a simulation time of 350 ns. The highest thickness was observed in the presence of CAGE (50% v/v), indicating greater IL intercalation within the lipid bilayer. Similar thickness was noted for the water and CAPA (50% v/v) groups, while CAGE (25% v/v) with CAPA (25% v/v) led to a higher thickness (FIG. 21D). The MD simulation snapshots highlight the dynamics of interactions of the individual ionic species in the IL with phospholipid membrane. Conclusive intercalation of the ionic species of the IL combination with the bilayer was detected (FIG. 21B). Furthermore, upon visualizing the trajectories of the individual ionic species within the CAGE+CAPA simulation, reduced mobility of geranic acid relative to phenylpropanoic acid was observed (FIGS. 26A-26B). When focusing on an IL aggregate that consists of all three IL species (choline, geranic acid, and phenylpropanoic acid), it was observed that each geranic acid molecule tends to remain in contact with the aggregate over the course of the simulation, while choline and phenylpropanoic acid are able to move between both the aggregate of heterogeneous species and the bulk solvent making up the rest of the system. This increase in mobility likely causes a change in the distribution of local viscosities across the system. When visualizing the head groups of lipids, which are in contact with the aggregate, the head groups were observed to occupy a larger area per lipid. This is demonstrated by a more “spread out” distribution of individual molecular trajectories within the area of the IL aggregate. This expansion of space between the lipids is caused by intercalation of the IL with the membrane and subsequent displacement of the lipid species. As the aggregation induces localization of the effects of IL on the bilayer membrane, it is likely that aggregation, with low constituent turnover with the bulk solvent, may lead to uneven membrane disruption as well as differences in the local viscosity. This heterogeneous distribution of membrane disruption may account for the wide distribution of area per lipid values seen over the course of the simulations in CAGE when compared with the other IL systems (FIG. 21E). Overall, these results signify the contribution of aggregate turnover for ILs in translocating RNA across lipid bilayers.

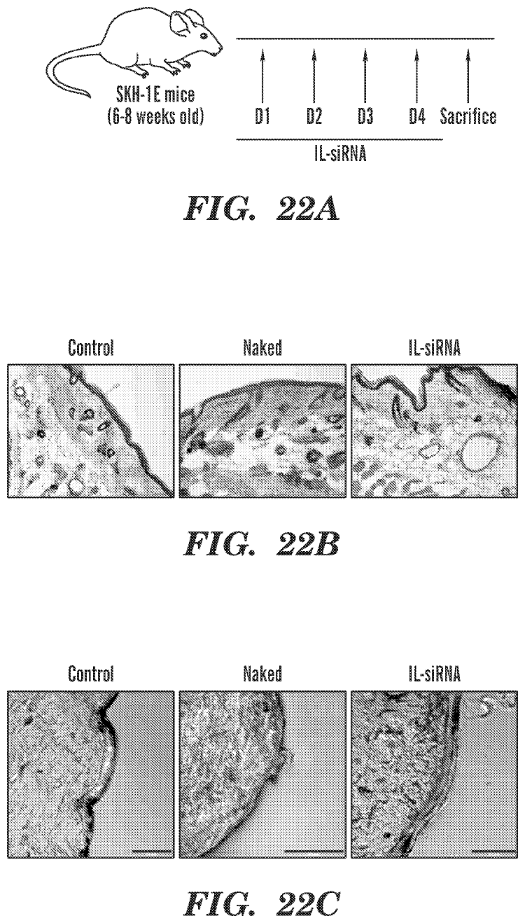

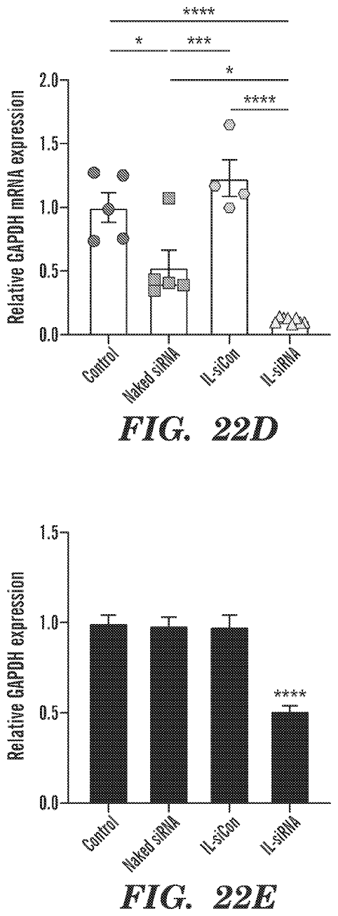

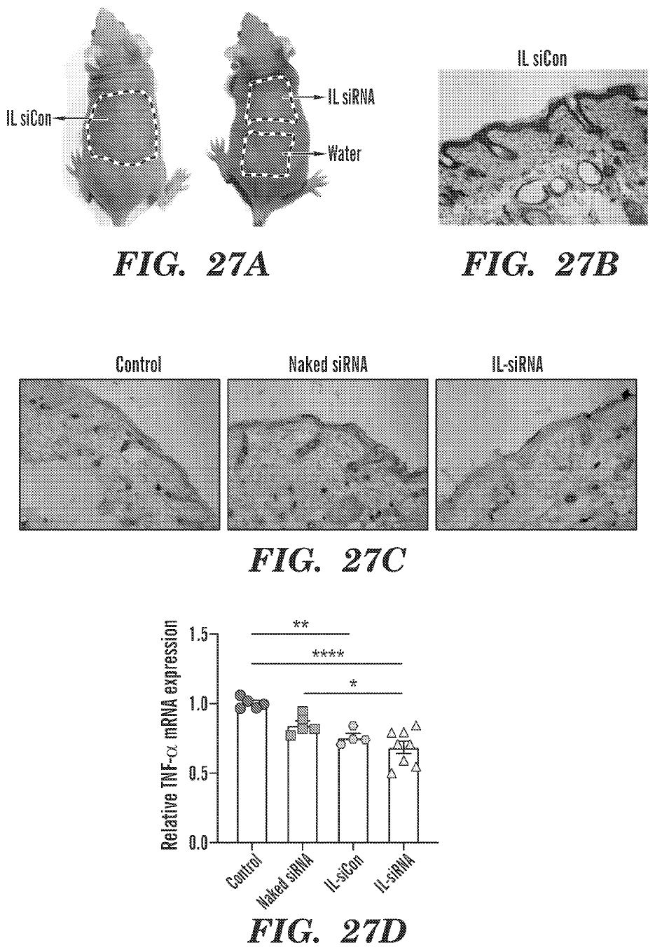

Biocompatibility of ILs in mice. The optimized CAGE+CAPA IL formulation was evaluated for toxicity in vivo in mice. IL-glyceraldehyde-3-phosphate dehydrogenase (GAPDH) siRNA formulation (25 l) was applied topically for four consecutive days to the dorsal skin of SKH-1 elite (SKH-1E) hairless mice (FIG. 22A). No signs of inflammation, redness, and/or irritation were observed for the IL-treated animals (FIGS. 27A-27D). Skin tissue was further harvested, and sections were cut and stained for histopathology and toxicology markers. Groups treated with the IL formulation exhibited no signs of epidermal thickening and keratinocyte hyperproliferation and were equivalent to the untreated and/or naked siRNA-treated animals (FIG. 22B and FIGS. 27A-27D). TNF-α gene expression levels were also tested in healthy mice. Animals treated with naked siRNA were statistically equivalent to the untreated animals. Mice treated with IL-GAPDH siRNA and IL-siCon (control siRNA used for subsequent experiments) demonstrated slightly lower TNF-α mRNA transcripts compared with the untreated group (FIGS. 27A-27D).

IL-siRNA penetration and GAPDH silencing in healthy mice. Cy5 fluorescence within the epidermis was measured in healthy mice following transdermal application for four consecutive days. Confocal images revealed a marked increase in Cy5 fluorescence in the epidermis for the IL-treated group compared with the naked siRNA in mice (FIG. 22C). Upon determining the GAPDH gene silencing efficiency using quantitative polymerase chain reaction (qPCR), the expression levels of GAPDH were found to be reduced 4.5- and 8.6-fold for the IL-siRNA-treated group in contrast to the naked siRNA and untreated mice, respectively (FIG. 22D). A slight decrease in the GAPDH mRNA expression was also observed for the naked siRNA-treated group. Consecutively, it was necessary to ascertain if the change in GAPDH mRNA expression translated into protein reduction. Consistent with the gene knockdown results, the IL-siRNA-treated group demonstrated a statistically significant decay (˜2-fold) in the GAPDH protein expression compared with all the other treatment groups (FIG. 22E). The reduced GAPDH mRNA expression for the naked siRNA-treated group did not down-regulate GAPDH protein expression.

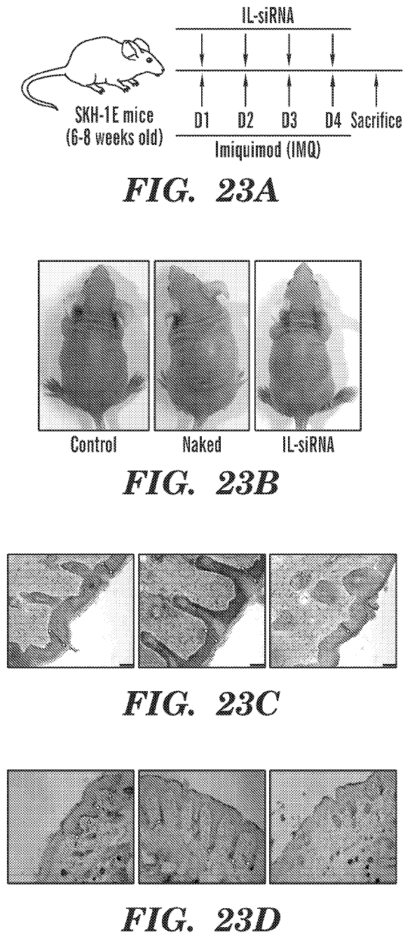

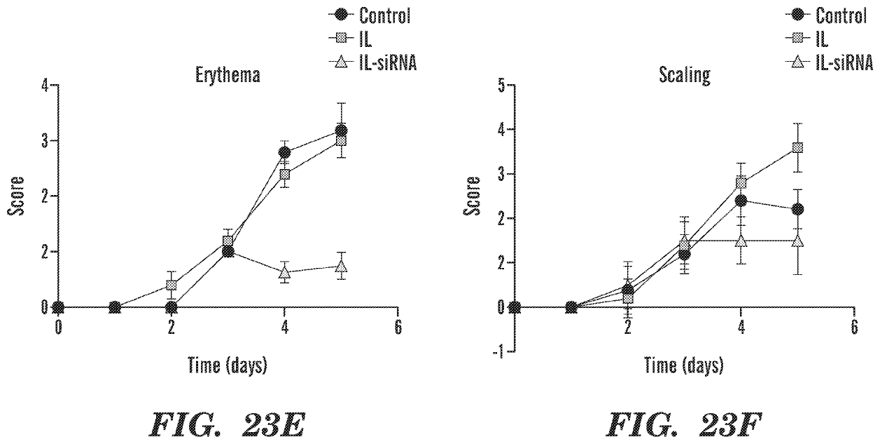

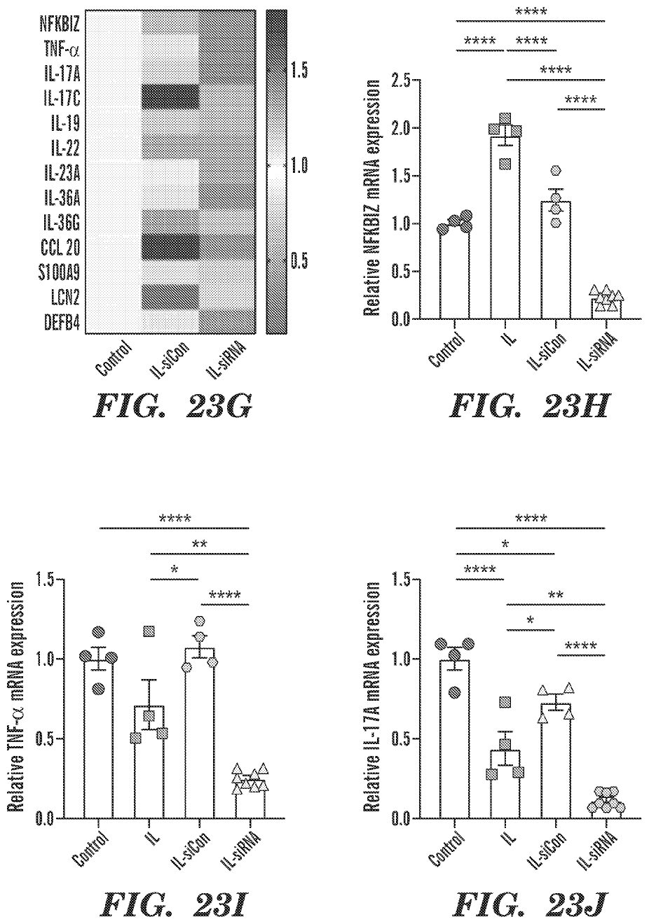

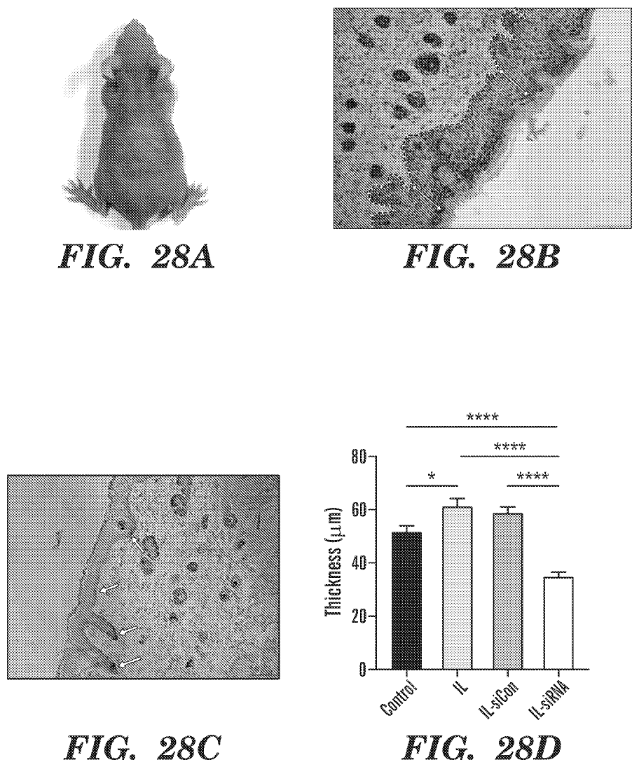

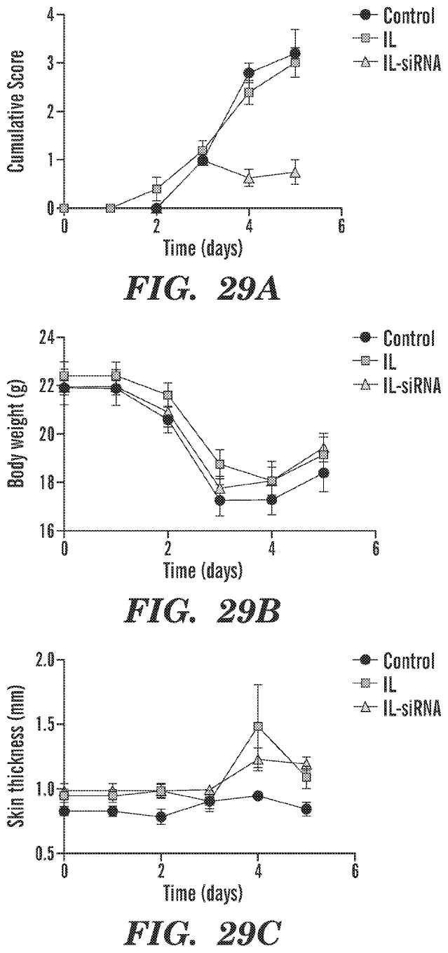

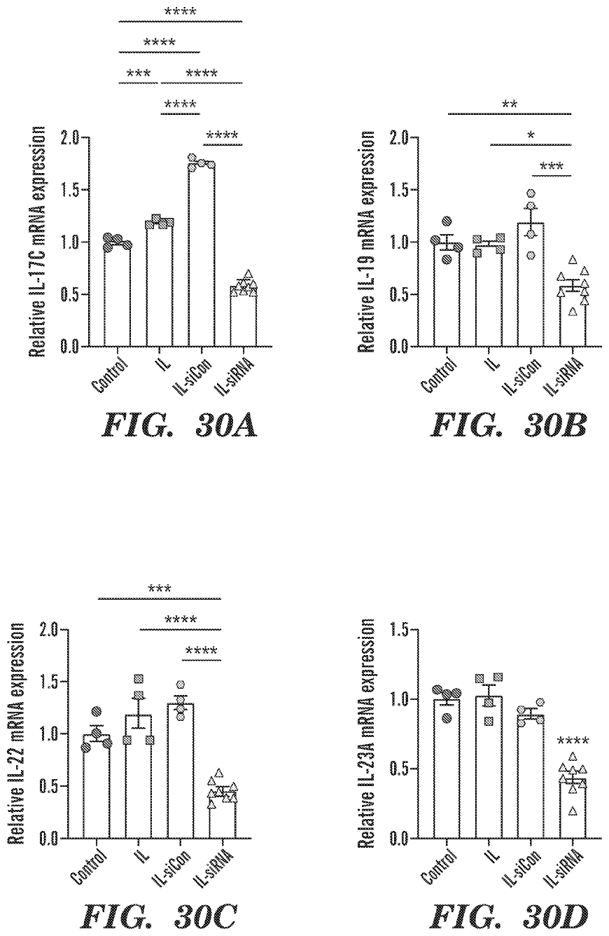

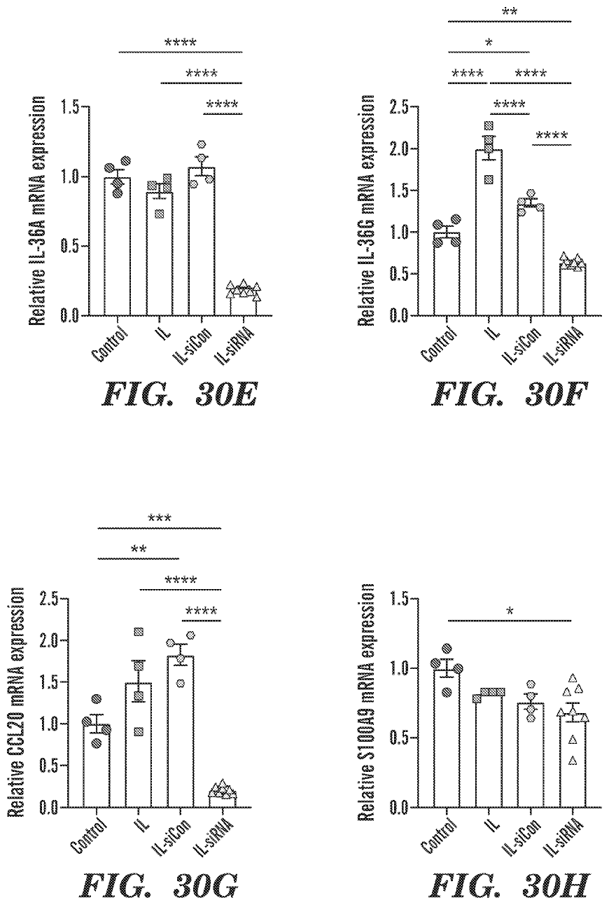

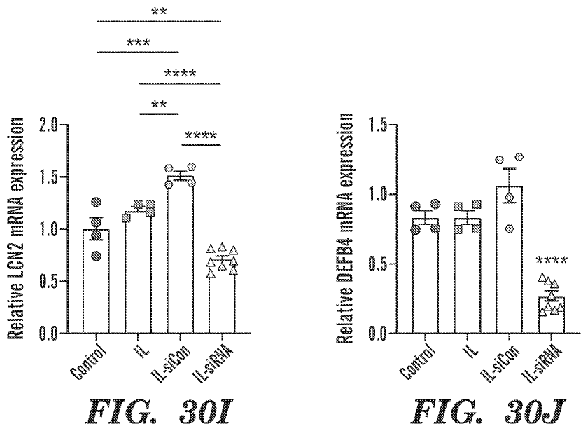

Local NFKBIZ silencing in the skin inhibits imiquimod-induced psoriasis. The ability of NFKBIZ siRNA to treat psoriasis was tested using CAGE+CAPA as a topical formulation. Following induction of psoriasis and topical application of IL-NFKBIZ siRNA formulation (FIG. 23A), skin tissue was harvested and analyzed. Macroscopically, local knockdown of NFKBIZ in the dorsal skin markedly reduced imiquimod-induced inflammation, showing reduced erythema and scaling in the area where IL-NFKBIZ siRNA was applied compared with the untreated, IL-treated, and IL-siCon-treated groups (FIG. 23B and FIGS. 28A-28D). Hematoxylin and eosin (H&E) staining of skin sections from the mice revealed that the knockdown of NFKBIZ by IL-siRNA reduced epidermal thickening, acanthosis, hyperkeratosis, and club-shaped rete ridges (FIG. 23C and FIGS. 28A-28D). Likewise, immunohistochemistry (IHC) analysis revealed hyperproliferation of keratinocytes in the untreated, IL-treated, and IL-siCon-treated groups, whereas the group treated with IL-NFKBIZ siRNA exhibited lack of keratinocyte proliferation (Ki67 staining) (FIG. 23D and FIGS. 28A-28D). The common characteristic features of imiquimod-induced skin inflammation (erythema and scaling) were scored daily throughout the induction/application period. Individual scores for erythema and scaling demonstrated fair reduction starting from day 3 with topical IL-siRNA application (FIGS. 23E-23F). Maximum cumulative scores were obtained for the untreated and IL-treated groups and were markedly lowered in the IL-siRNA-treated group (FIGS. 29A-29C). Double skin-fold thickness (DSFT) for measuring skin thickness did not yield major differences between the groups (FIGS. 29A-29C). In addition, the heat map and mRNA analyses indicated a substantial reduction in expression of NFKBIZ and other psoriasis-related gene products in comparison with the untreated and IL-siCon-treated groups (FIGS. 23G-23J and FIGS. 30A-30J). Upon IL-siCon treatment, most genes were up-regulated, including NFKBIZ, TNF-α, cytokines (IL-17C, IL-19, IL-22, IL-36A, and IL-36G), chemokines (CCL20), and antimicrobial proteins (LCN2 and DEFB4) (FIG. 23G). Some down-regulation of TNF-α and IL-17A mRNA expression was observed in healthy mice upon treatment with IL alone (FIGS. 23I-23J).

### DISCUSSION

Limited understanding of key inflammatory signaling pathway regulators and the chronological order of the underlying mechanisms presents a challenge in the treatment of psoriasis. Signaling pathways including NF-κB, Janus kinase (JAK)/signal transducer and activator of transcription (STAT), and p38 mitogen-activated protein kinase have recently been found to play a major role in the pathogenesis of this complex disease (31). NFKBIZ, a gene encoding IκBζ, is a crucial transcriptional coactivator mediating downstream effects of an array of specific inflammatory cytokines and is particularly imperative in the light of recent findings by Johansen et al. (6) and Müller et al. (12), which indicated IκBζ to be a key modulator of IL-17A, IL-23, and IL-36 (32). Thus, targeting NFKBIZ/IκBζ to inhibit proinflammatory signaling pathways and production of psoriasis-related gene products is a viable strategy for psoriasis treatment. Clinically, antibodies targeting TNF-α and IL-17A have shown promise in meeting the primary endpoints and improving the disease condition (33). However, as biologics, these antibodies have challenges of potential systemic toxicity, generation of anti-antibodies, and high cost.

Described herein is an IL combination capable of improving epidermal accumulation and delivery of RNA through skin. The inventors hypothesized that a combination of ILs would stabilize the siRNA and, at the same time, would improve its penetration. This hypothesis was validated in an imiquimod-induced psoriasis-like skin inflammation model that resembles plaque-type psoriasis in humans. Topical application of IL-siRNA for four consecutive days generated substantial reduction in the levels of inflammatory cytokines and an array of psoriasis-related gene products.

CAGE+CAPA IL formulation offers several advantages over other transdermal drug delivery systems. The components of the IL formulation, choline bicarbonate, geranic acid, and phenylpropanoic acid, have been proven safe or GRAS (generally recognized as safe) chemicals and provide a strong foundation for the safety of ILs. In addition, simple synthesis and scale-up processes, high solvating power, and tunability offer additional advantages over other volatile organic solvents. This system is particularly suitable for transdermal delivery of nucleic acids due to both its complex intercalation between the stacked RNA base pairs and aromatic rings of the IL, and enhanced interaction with the lipid bilayer.

These results demonstrate that ILs can complex with nucleic acids without compromising the bioactivity, thus making them ideal for transdermal drug delivery. The salt metathesis or anion exchange reaction for IL synthesis is particularly advantageous because it does not require integration of harsh organic solvents for siRNA delivery. The individual IL components can be modulated to interact with nearly any nucleic acid based on the binding characteristics and molecular mechanism of interactions.

Tunable ion stoichiometry and physicochemical properties are other key features of IL-based systems. Previous work has indicated the role of interionic interactions in solvation and partitioning of the active ingredient into the skin (34). In addition, Chandran et al. (35) have demonstrated the importance of electrostatic interactions and groove binding associations of ILs in DNA stability. Hitherto, the role of ILs in improving the stability and solvation of siRNA has not been comprehensively explored. The work presented herein systematically varied the anionic component of the IL with structural similarity to geranic acid and/or containing an aromatic ring at a stoichiometry ratio of 1:2 and developed a cholinium-based IL library. It was observed that the anions of the ILs that contained aromatic rings generally solidified or formed a gel at RT except phenylpropanoic acid. Excellent siRNA stability was observed in the presence of CAVA, CAPA, and CAGE+CAPA in comparison to other ILs and combinations, possibly due to superior interactions with the siRNA. The IL combination CAGE+CAPA generated the highest epidermal accumulation of siRNA, notably higher than any individual ILs and/or combination.

The best performing IL combination identified in this study, CAGE+CAPA, demonstrated consistent distribution of the three ionic species through MD simulations, indicating improved molecular mobility and lower viscosity contributing to enhanced solvation effects. Furthermore, MD simulation snapshots revealed close association of phenylpropanoic acid with the RNA molecules, which could be possibly attributed to a combination of hydrophobic and polar interactions, 7-7 stacking, and/or intercalation between stacked RNA base pairs, leading to enhanced RNA stability. RGYR and RMSD measurements obtained from simulations over the course of 500 ns further confirmed improved IL-RNA interactions.

It is also important to understand the magnitude of IL-mediated lipid bilayer modulation. MD simulations revealed the crucial role of aggregation of ionic species in improving membrane permeation with the highest bilayer thickness obtained for CAGE (50% v/v) followed by CAGE+CAPA. Such observations from the simulations further establish geranic acid as the main driver in the translocation of the IL combination through the lipid bilayers, which is consistent with experimental results. While it seems that phenylpropanoic acid has a minor role in improving bilayer permeation by lowering the local viscosity of the overall IL system, these results also indicate that it is also responsible for fluidizing the membrane with the formation of dynamic pores. It was earlier reported that deprotonated aromatic carboxylic acids, such as phenylpropanoic acid, permeate bilayers several orders of magnitude faster than that expected from the pH partition hypothesis, and their permeation is fully controlled by the anions at the physiological pH (36). It is contemplated that these ILs assist in crossing the cellular barriers to deliver siRNA into the cytosolic compartments.

To assess biocompatibility of CAGE+CAPA, a histological evaluation of skin was conducted on the fifth day, which coincided with the total duration of topical application. No macroscopic changes in the skin structure, epidermal thickening, and keratinocyte proliferation in the IL-treated groups were observed. Further investigation of inflammatory cytokine levels did not reveal any statistically significant increment in TNF-α mRNA compared with the untreated groups. Some of the IL-treated groups demonstrated a decrease in the TNF-α mRNA levels, which might be possibly due to the presence of IL. Marked inhibition of GAPDH mRNA and protein expression was observed in the IL-GAPDH siRNA-treated groups.

NFKBIZ has been previously demonstrated to play a crucial role in the gene transcription of several proinflammatory cytokines and antimicrobial peptides responsible for the pathogenesis of psoriasis (6, 12). Using an imiquimod-induced psoriasis model, it was demonstrated that local silencing of NFKBIZ following topical application of IL-NFKBIZ siRNA formulation impaired expression of psoriasis-related gene products under in vivo conditions. IL-siRNA-treated mice exhibited substantially reduced skin pathology including reduced erythema and scaling, less epidermal thickening, and keratinocyte proliferation. The local increase in mRNA levels of some of the inflammatory cytokines and related gene products for the IL-siCon- and IL-treated groups in comparison with the untreated group could be attributed to imiquimod. Local silencing of NFKBIZ resulted in a strong inhibition of crucial proinflammatory cytokine mRNA levels including IL-17A, IL-23, and IL-36. The downstream effects of local NFKBIZ silencing were also validated and are consistent with the previously reported effects of intradermal injection of IκBζ siRNA (6). Because mouse skin is generally much more permeable than human skin, detailed studies of quantification of skin penetration were not performed in vivo.

In summary, provided herein is a transdermal IL platform capable of delivering RNA to the epidermis. The platform is combined with an array of gene screening to support NFKBIZ as a key signaling target gene in psoriasis treatment. The IL formulation retained the bioactivity of the siRNA and generated notable target gene abrogation upon topical application in an imiquimod-induced psoriasis-like skin inflammation model. The optimized IL formulation did not show toxicity and is acceptable for repeated applications. This platform is amenable to broad applications to nucleic acids and can be easily manufactured and scaled up. This platform can empower transdermal drug delivery for the treatment of dermatological conditions and help augmenting long-term therapeutic efficacy by targeting such common mediators.

Materials and Methods

Skin-penetrating IL-RNA complexes. The cholinium-based IL library was synthesized as described previously (26). Briefly, the cation, choline bicarbonate, and various anions were mixed at a 1:2 ratio to prepare ILs following salt metathesis reaction. The anions were dissolved in a minimum volume of ultrapure water or ethanol/methanol based on the solubility and were reacted with choline bicarbonate at 40° C. for 24 hours. The resulting IL solution was dried using a rotary evaporator at 20 mbar at 60° C. for 2 hours. The residual water was removed in a vacuum oven at 60° C. for 48 hours. The ILs that were viscous at RT were characterized via NMR with dimethyl sulfoxide (DMSO)-d6 on an Agilent DD2 600-MHz spectrometer (Supplementary Materials and Methods). ILs were mixed with RNA (100 μM) at a volumetric ratio of 1:1 and incubated for 30 min at RT. The RNA-IL solutions (1 ml) were dialyzed against 10 mM sodium phosphate buffer for 72 hours using Dialysis Cassettes (10,000 molecular weight cutoff, Invitrogen). The concentration of RNA was confirmed and normalized using a NanoDrop instrument (Thermo Fisher Scientific). The stability of the RNA in the IL solution was determined using CD and gel electrophoresis.

MD simulation studies. MD simulations were performed using OpenMM MD package and the AMBER force fields ff14SB and GAFF. Three-dimensional SD files for each of the IL species were downloaded from PubChem and parameterized with Antechamber before preparing simulation input topologies with LEaP. To generate starting coordinates for the lipid membrane simulations, PACKMOL was used to build a bilayer consisting of 100 phosphatidylcholine (POPC) molecules for each of the leaflets. The remaining contents of a 60-Å cube consisted of ˜1:1 water (TIP3P) and IL, charge balanced with Na+ and Cl−. A 500-ns simulation was performed for each of the systems under periodic boundary conditions. For the simulations of siRNA, a helical starting structure for the nucleic acid was generated with Avogadro (37) before being placed in a simulation box consisting of ˜1:1 water and IL for simulation under periodic boundary conditions for 350 ns. Analysis of MD trajectories was performed using the python library MDAnalysis for RGYR and RMSD of siRNA. Visual molecular dynamics (38) plugin MEMBPLUGIN (39) was used to perform analysis of membrane trajectories.

Skin penetration studies. Skin penetration studies were performed using porcine skin in FDCs, as described previously (40). A total volume of 20 μl of Cy5-labeled RNA (50 μM) in IL solutions was applied to the porcine skin surface and was incubated at 40° C. for 24 hours under occlusive conditions with moderate stirring. The skin permeability of RNA was visualized and quantified using confocal microscopy and tape-stripping techniques, respectively.

Animal studies. All animal studies were performed at the Harvard John A. Paulson School of Engineering and Applied Sciences, Harvard University. Procedures and studies conducted were approved by the Institutional Animal Care and Use Committee of the Faculty of Arts and Sciences, Harvard University, and were consistent with all applicable regulations. ILs carrying GAPDH (custom siRNA, sense seq: 5′-GUGUGAACCACGAGAAAUAUU-3″ (SEQ ID NO: 5), antisense seq: 5′-AAUAUUUCUCGUGGUUCACAC-3′ (SEQ ID NO: 6); Dharmacon), siCon (catalog no. D-001810-02-50; Dharmacon), and NFKBIZ siRNAs (catalog no. J-040680-06-0050; Dharmacon) were applied topically to healthy and imiquimod-treated SKH-1E hairless mice (Charles River), respectively. A blind scoring system similar to the human Psoriasis Area and Severity Index (PASI) score was used to measure the degree of severity, erythema, and scaling on the back of mice. In addition, skin thickness was monitored by the DSFT of dorsal skin of the mice with caliper measurements throughout the disease induction and treatment period.

Quantification of mRNA transcripts. Flash-frozen skin tissues were pulverized to form a powder and homogenized in QIAzol Lysis Reagent to prepare the tissue lysates for qPCR. The mRNA levels were quantified and normalized following the manufacturer's protocol. The relative abundance of mRNA transcripts and silencing in treated groups was normalized to the housekeeper gene (0-actin). The mean normalized siRNA treatment values were then plotted with their SEM.

Statistical analysis. One-way analysis of variance (ANOVA) and statistical analyses were performed using GraphPad Prism software (GraphPad Software Inc.). Results are depicted as average±SEM. Two-tailed Student's t test was used for comparison between two groups. Parametric data were analyzed by one-way ANOVA followed by Tukey's honestly significant difference (HSD) post hoc tests. Kruskal-Wallis tests were performed for nonparametric data. Statistical tests are indicated in the figures. P<0.05 was considered statistically significant.

Circular dichroism Circular dichroism measurements of dialyzed RNA samples were recorded at 15° C. using a 1 cm pathlength quartz cell (Hellma 100-10-40, style 100-QS), in the Jasco J-815 spectropolarimeter equipped with a PFD-425S thermal controller unit at the Center for Macromolecular Interactions (CMI), Harvard Medical School. RNA concentrations were normalized in 10 mM sodium phosphate buffer and incubated for 30 mins at RT to ensure reduction and equilibrium, and then loaded into quartz cuvettes. Near-UV spectra were recorded from 200 nm to 310 nm at 20° C. by averaging 5 scans at 0.1 nm intervals for each sample. Spectrum Manager 2 was used to subtract the baseline and the spectra were plotted as molar ellipticity, [0] (deg.cm2.dmol-1).

Nuclear magnetic resonance All NMR experiments were performed at 0 to 50° C. on an Agilent DD2-600 NMR equipped with 5 mm inverse triple-resonance nanoprobe with 1200 (H), 100 (C), 2000 (H) sensitivity at the Harvard CCB Laukien-Purcell Instrumentation Center, Magnetic Resonance Laboratory. Each IL formulation was characterized by 1H NMR by placing dried IL into an NMR tube containing a co-axial insert filled with DMSO-d6. NMR data were processed and analyzed using Mnova qNMR v1.0.

Gel Electrophoresis In order to determine the stability of RNA in IL solutions, the dialyzed RNA samples were separated in 1% Agarose gels (containing 0.01% v/v 10,000× GelRed Nucleic Acid Stain, in 1×TBE). Agarose solution was prepared by dissolving in 1×TBE and heated in microwave at 60° C. for 10 min until dissolved completely. The agarose solution was poured into the casting stand and a 10-well comb was placed to generate wells that are 1.5 mm deep. The melted agarose was allowed to cool for 30 mins at RT for polymerization. The chamber was filled with 1×TBE buffer solution to a height of 1.5 cm above the gel surface. RNA samples were premixed with the agarose gel loading dye (6×) prior to loading 2 μL of samples into the wells from left to the right. The power supply was activated as soon as all wells were filled, to avoid initial diffusion of the dye into the gel. The samples were run at 100V for 40 mins and were imaged using Azure c300 (Azure Biosystems) with cSeries Capture software at the Bauer Core Facility, Harvard University.

Porcine skin penetration ex-vivo studies Porcine skin studies were carried out in Franz diffusion cells (FDC) with penetration area of 1.77 cm². The porcine skin was obtained from Lampire Biological Laboratories, Pipersville, PA, USA. Briefly skins were thawed, hairs were trimmed, and washed with phosphate buffer saline (PBS, pH 7.4). A 36 mm punch was used to cut out a disc of the skin and a scalpel was used to get rid of the connective tissues and subcutaneous fat layers. The skin (roughly 0.5 mm thick) was placed on the diffusion cell with the stratum corneum (SC) layer facing upwards. The acceptor component of the cell was filled with PBS (˜12 mL) and equipped with a magnetic stirrer bar. 1 mL PBS was added to the donor chamber and the conductivity was measured using a waveform generator (Agilent 33120) and voltmeter (Fluke 87 True RMS Multimeter) at a frequency of 100 Hz and amplitude, 100 mV. Only skin samples with a measured transepidermal conductivity of less than 10 ρA were used for further studies. The cells were kept in an oven at 37° C. to warm up. The donor compartment was left for 5 mins before applying 20 μL of Cy5-labelled siRNA-IL (siRNA 50 μM) solution on top of the skin ensuring full coverage. The donor chamber and side-arm of the cells were sealed with parafilm/foil and eppendorf respectively to reduce evaporation and were incubated at 37° C. for 24 h on a stirrer plate. Following incubation, the skin was removed from the cell, washed gently with PBS and were further analyzed using tape-stripping and confocal microscopy.

Cryosectioning After removing the skin from the cell and washing in PBS, skin tissues were flash frozen (up to 2.0 cm in diameter) in OCT (Sakura Finetek, USA) using a suitable tissue mold. Thin sections of the skin (15-20 m) corresponding to the application area were cut using a Leica Cryostat CM1850 (Leica, Buffalo Grove, IL) at −20° C. The cut sections were transferred immediately to glass slides (kept at RT) by touching the slides to the sections and were further analyzed.

Confocal microscopy Following sectioning in the cryostat, skin sections were covered with cover slips. Microscopy was performed on Zeiss 710 Confocal system equipped with Zeiss Axio Imager Z2 microscope with Colibri FL Illumination and CoolSnapnHQ2 camera. The sections were imaged with 40× air 1.2 numerical aperture objective and Ar laser 633 nm for red fluorescent Cy5. Images were processed using a java-based image processing program, ImageJ/FIJI. All image acquisition and processing were executed under identical conditions for control and test samples.

Tape-stripping After removal of the skin from the cell and washing in PBS, the SC was stripped from the epidermis using an adhesive tape up to ten layers (SC1, SC2-5, SC6-10). Following SC removal, the epidermis was separated from the dermis using a surgical sterile scalpel and a third of dermis (by area) was removed by punching with 4 mm three times. Each layer was collected separately in glass vials containing 1 mL of PBS/methanol (1:1) mixture and was left to shake overnight to extract the Cy5-siRNA from the skin layers, which was further analyzed using a plate reader (Tecan Safire, AG, Switzerland) on a 96-well plate at an excitation wavelength of 633 nm and emission wavelength of 665 nm.

Mice and treatments Female SKH-1E hairless mice (6-8 weeks old) were purchased from Charles River Laboratories (MA, USA). The animals were kept in a controlled temperature (24 to 26° C.), a daily 12:12 h light/dark cycle and food and water ad libitum. Experiments were performed according to the approved protocols by the Institutional Animal Care and Use Committee of the Faculty of Arts and Sciences, Harvard University. Healthy mice were treated with 25 μL of GAPDH siRNA (50 μM)-IL formulation each day for four consecutive days.

For the psoriasis model, mice were treated with a daily dose of freshly prepared 25 μL of 50 μM NFKBIZ siRNA-IL formulation to the dorsal skin in the morning and air dried. Six hours later, 62.5 mg 5% imiquimod cream (Aldara; Perrigo Co.) obtained from Patterson Veterinary, CO, USA was applied to the same region. Both the IL-siRNA and imiquimod treatments were continued for 4 days. The skin thickness of the dorsal skin was assessed daily by the double skin-fold thickness (DSFT) using an electronic digital Vernier caliper. Erythema and scaling were scored blindly using human Psoriasis Area and Severity Index (PASI) scoring system daily on a scale from 0 (no alteration) to 4 (very distinct alteration) as previously described. The single scores were combined, resulting in a theoretical maximal cumulative score of 8. On day 5, animals were euthanized in a CO2 chamber and the treated dorsal skins (skin area ˜4 cm²) were harvested and collected for histology, and qPCR.

ELISA For semi-quantitative measurement of GAPDH protein in mouse cells following GAPDH siRNA treatment, GAPDH SimpleStep ELISA Kit (ab176642, Abcam) was employed. Briefly, 200 mg of harvested frozen skin was pulverized using mortar and pestle to form a powder and homogenized in chilled 0.5 mL 1× cell extraction buffer. The lysates were incubated on ice for 20 mins and centrifuged at 18000×g for 20 min at 4° C. The supernatants were collected in clean tubes and the protein concentrations in each sample were quantified immediately using Nanodrop. The samples were diluted to 20 mg/mL protein concentrations using 1× cell extraction buffer. The plate strips were prepared following manufacturer's protocol and protein levels were measured using a microplate reader (Biotec Synergy 2, USA) at 450 nm.

qPCR After frozen tissues were pulverized to form a powder, tissue lysates were homogenized in 700 μL QIAzol Lysis Reagent and the total RNA was extracted using Qiagen miRNeasy Mini Kit (217004) according to the manufacturer's protocol. The mRNA levels were normalized and was reverse transcribed using Biorad iScript™ Reverse Transcription Supermix (1708841) to yield cDNA. Real-time reverse-transcription PCR was performed on the obtained cDNA with SsoFast EvaGreen Supermix (172-5211). Triplicate reactions for the gene of interest and the endogenous control (D-Actin) were performed separately on the same cDNA samples on a Biorad CFX 96 instrument. The following primer sequences of the mouse NFKBIZ, TNF-α, IL-17A, IL-17C, IL-19, IL-22, IL-23A, IL-36A, IL-36G, CCL20, S100A9, LCN2, and DEFB4 genes were used: GAPDH (Forward: 5′-ACCACAGTCCATGCCATCAC-3′ (SEQ ID NO: 7); Reverse: 5′-TCCACCACCCTGTTGCTGTA-3′ (SEQ ID NO: 8)), NFKBIZ (Forward: 5′-TATCGGGTGACACAGTTGGA-3′ (SEQ ID NO: 9); Reverse: 5′-TGAATGGACTTCCCCTTCAG-3′ (SEQ ID NO: 10)), TNF-α(Forward: 5′-GGCAGGTTCTGTCCCTTTCAC-3′ (SEQ ID NO: 11); Reverse: 5′-TTCTGTGCTCATGGTGTCTTTTCT-3′ (SEQ ID NO: 12)), IL-17A (Forward: 5′-ATGAGTGCCGACAAACAACG-3′ (SEQ ID NO: 13); Reverse: 5′-GTGACGTGGAACGGTTGAGG-3′ (SEQ ID NO: 14)), IL-17C (Forward: 5′-CTGGAAGCTGACACTCACGA-3′ (SEQ ID NO: 15); Reverse: 5′-GGTAGCGGTTCTCATCTGTG-3′ (SEQ ID NO: 16)), IL-19 (Forward: 5′-TTCCACGAGATCAAGAGAGC-3′ (SEQ ID NO: 17); Reverse: 5′-TCTACACCTGTTCCGCTGAG-3′ (SEQ ID NO: 18)), IL-22 (Forward: 5′-TTGAGGTGTCCAACTTCCAGCA-3′ (SEQ ID NO: 19); Reverse: 5′-GCATAGGTAGCCAGAGCCAG-3′ (SEQ ID NO: 20)), IL-23A (Forward: 5′-TGGCATCGAGAAACTGTGAGA-3′ (SEQ ID NO: 21); Reverse: 5′-TCAGTTCGTATTGGTAGTCCTGTTA-3′ (SEQ ID NO: 22)), IL-36A (Forward: 5′-AGTGGGTGTAGTTCTGTAGTGTGC-3′ (SEQ ID NO: 23); Reverse: 5′-GTTCGTCTCAAGAGTGTCCAGATAT-3′ (SEQ ID NO: 24)), IL-36G (Forward: 5′-CACAGATGAGAACCGCTACCC-3′ (SEQ ID NO: 25); Reverse: 5′-GCGGATGAACTCGGTGTGGAA-3′ (SEQ ID NO: 26)), CCL20 (Forward: 5′-GTGGGTTTCACAAGACAGATG-3′ (SEQ ID NO: 27); Reverse: 5′-TTTTCACCCAGTTCTGCTTTG-3′ (SEQ ID NO: 28)), S100A9 (Forward: 5′-CCTTCTCAGATGGAGCGCAG-3′ (SEQ ID NO: 29); Reverse: 5′-TGTCCAGGTCCTCCATGATG-3′ (SEQ ID NO: 30)), LCN2 (Forward: 5′-GGACCAGGGCTGTCGCTACT-3′ (SEQ ID NO: 31); Reverse: 5′-GGATCCCGATGGCTAGAGCA-3′ (SEQ ID NO: 32)) and DEFB4/mBD4 (Forward: 5′-AGGGAAGGATGAGATTAAGACTGG-3′ (SEQ ID NO: 33); Reverse: 5′-CTTGCTGGTTCTTCGTCTTTT-3′ (SEQ ID NO: 34). Primers for the housekeeping gene, R-Actin (Forward: 5′-CGGTTCCGATGCCCTGAGGCTCTT-3′ (SEQ ID NO: 35); Reverse: 5′-CGTCACACTTCATGATGGAATTGA-3′ (SEQ ID NO: 36)). The specificities of the primers were verified, and amplicon specificity was monitored by melting curve analysis. For each genomic sequence evaluated, a ΔCt value was calculated for each sample by subtracting the Ct value of the treated sample from the Ct value obtained for the untreated/control group. Calculating 2\hat{ }ΔΔCt yielded the relative amount of PCR product (relative enrichment).

Histopathology and Immunohistochemistry Sections (15-20 m) from OCT embedded tissues were stained with hematoxylin and eosin and evaluated by light microscopy. For Ki67 immunohistochemistry, sections were heated at 100 in citrate buffer (pH 6.0) for 30 mins for antigen retrieval. Sections were incubated with anti-ki67 primary antibody (rabbit anti-mouse monoclonal; 1:1000 dilution; ab16667, Abcam, Cambridge, UK) overnight at 4° C. and later with peroxidase coupled anti-rabbit IgG secondary antibody for 30 mins. Sections were stained with DAB, counterstained with hematoxylin and evaluated using light microscopy (Olympus BX53 microscope with Olympus camera). Epidermal thicknesses were measured for control and test samples using ImageJ/FIJI software.

### REFERENCES AND NOTES

1. E. A. Brezinski, et al., Economic burden of psoriasis in the United States: A systematic review. JAMA Dermatol. 151, 651-658 (2015).
2. S. T. Smale et al., Hierarchies of NF-κB target-gene regulation. Nat. Immunol. 12, 689-694 (2011).
3. S. Yan, et al., NF-κB-induced microRNA-31 promotes epidermal hyperplasia by repressing protein phosphatase 6 in psoriasis. Nat. Commun. 6, 7652 (2015).
4. N. A. Pabon, et al., A network-centric approach to drugging TNF-induced NF-κB signaling. Nat. Commun. 10, 860 (2019).
5. M. S. Veilleux, et al., Biologics in patients with skin diseases. J. Allergy Clin. Immunol. 139, 1423-1430 (2017).
6. C. Johansen, et al., IκBζ is a key driver in the development of psoriasis. Proc. Natl. Acad. Sci. U.S.A. 112, E5825-E5833 (2015).
7. S. Sano, et al., Stat3 links activated keratinocytes and immunocytes required for development of psoriasis in a novel transgenic mouse model. Nat. Med. 11, 43-49 (2005).
8. L. C. Tsoi, et al., Enhanced meta-analysis and replication studies identify five new psoriasis susceptibility loci. Nat. Commun. 6, 7001 (2015).
9. S. L. Gaffen, et al., The IL-23-IL-17 immune axis: From mechanisms to therapeutic testing. Nat. Rev. Immunol. 14, 585-600 (2014).
10. N. J. Wilson, et al., Development, cytokine profile and function of human interleukin 17-producing helper T cells. Nat. Immunol. 8, 950-957 (2007).
11. Y. Zheng, et al., Interleukin-22, a TH17 cytokine, mediates IL-23-induced dermal inflammation and acanthosis. Nature 445, 648-651 (2007).
12. A. Miller, IκBζ is a key transcriptional regulator of IL-36-driven psoriasis-related gene expression in keratinocytes. Proc. Natl. Acad. Sci. U.S.A. 115, 10088-10093 (2018).
13. M. R. Prausnitz, Transdermal drug delivery. Nat. Biotechnol. 26, 1261-1268 (2008).
14. S. D. Roy, Transdermal delivery of narcotic analgesics: comparative permeabilities of narcotic analgesics through human cadaver skin. Pharm. Res. 6, 825-832 (1989).
15. M. R. Prausnitz, Current status and future potential of transdermal drug delivery. Nat. Rev. Drug Discov. 3, 115-124 (2004).
16. D. Zheng, Topical delivery of siRNA-based spherical nucleic acid nanoparticle conjugates for gene regulation. Proc. Natl. Acad. Sci. U.S.A. 109, 11975-11980 (2012).
17. C. Wiraja, Framework nucleic acids as programmable carrier for transdermal drug delivery. Nat. Commun. 10, 1147 (2019).
18. Y. Deng, Transdermal delivery of siRNA through microneedle array. Sci. Rep. 6, 21422 (2016).
19. G. Yang, Punching and electroporation for enhanced transdermal drug delivery. Theranostics 8, 3688-3690 (2018).
20. R. Ruan, Topical and targeted delivery of siRNAs to melanoma cells using a fusion peptide carrier. Sci. Rep. 6, 29159 (2016).
21. M. Chen, Topical delivery of siRNA into skin using SPACE-peptide carriers. J. Control. Release 179, 33-41 (2014).
22. C.-M. Lin, A simple, noninvasive and efficient method for transdermal delivery of siRNA. Arch. Dermatol. Res. 304, 139-144 (2012).
23. V. D. Thanik, Topical matrix-based siRNA silences local gene expression in a murine wound model. Gene Ther. 14, 1305-1308 (2007).
24. L. N. Kasiewicz, Lipid nanoparticles silence tumor necrosis factor α to improve wound healing in diabetic mice. Bioeng. Transl. Med. 4, 75-82 (2019).
25. C. Agatemor, Ionic liquids for addressing unmet needs in healthcare. Bioeng. Transl. Med. 3, 7-25 (2018).
26. M. Zakrewsky, Ionic liquids as a class of materials for transdermal delivery and pathogen neutralization. Proc. Natl. Acad. Sci. U.S.A. 111, 13313-13318 (2014).
27. Institute of Medicine (U.S.) Committee on Military Nutrition Research, in Food Components to Enhance Performance: An Evaluation of Potential Performance-Enhancing Food Components for Operational Rations, B. M. Marriott, Ed. (National Academies Press, Washington (DC, 1994).
28. Q. M. Qi, Mechanistic study of transdermal delivery of macromolecules assisted by ionic liquids. J. Control. Release 311-312, 162-169 (2019).
29. E. E. L. Tanner, Transdermal insulin delivery using choline-based ionic liquids (CAGE). J. Control. Release 286, 137-144 (2018).
30. A. Banerjee, Ionic liquids for oral insulin delivery. Proc. Natl. Acad. Sci. U.S.A. 115, 7296-7301 (2018).
31. M. A. Lowes, Immunology of psoriasis. Annu. Rev. Immunol. 32, 227-255 (2014).
32. S. K. Mahil, An analysis of IL-36 signature genes and individuals with IL1RL2 knockout mutations validates IL-36 as a psoriasis therapeutic target. Sci. Transl. Med. 9, eaan2514 (2017).
33. Trial watch: Targeting IL-17A shows broad promise in autoimmune diseases. Nat. Rev. Drug Discov. 9, 908 (2010).
34. E. E. L. Tanner, Design principles of ionic liquids for transdermal drug delivery. Adv. Mater. 31, e1901103 (2019).
35. A. Chandran, Groove binding mechanism of ionic liquids: A key factor in long-term stability of DNA in hydrated ionic liquids? J. Am. Chem. Soc. 134, 20330-20339 (2012).
36. A. V. Thomae, Permeation of aromatic carboxylic acids across lipid bilayers: The pH-partition hypothesis revisited. Biophys. J. 89, 1802-1811 (2005).
37. M. D. Hanwell, Avogadro: An advanced semantic chemical editor, visualization, and analysis platform. J Cheminform. 4, 17 (2012).
38. W. Humphrey, VMD: Visual molecular dynamics. J. Mol. Graph. 14, 33-38 (1996).
39. R. Guixà-González, MEMBPLUGIN: Studying membrane complexity in VMD. Bioinformatics 30, 1478-1480 (2014).
40. T. Hsu, Delivery of siRNA and other macromolecules into skin and cells using a peptide enhancer. Proc. Natl. Acad. Sci. U.S.A. 108, 15816-15821 (2011).

What is claimed herein is:

1. A composition comprising at least one ionic liquid comprising:
an anion which is at least one of:
a) a carboxylic acid which is not a fatty acid;
b) a carboxylic acid comprising an aliphatic chain of no more than 4 carbons;
c) an aromatic anion; and/or
d) an anion with a LogP of less than 1.0; and
a cation comprising a quaternary ammonium.

2. The composition of any of the preceding claims, wherein the anion has a LogP of less than 1.0 and is:
a. a carboxylic acid which is not a fatty acid;
b. carboxylic acid comprising an aliphatic chain of no more than 4 carbons; or
c. an aromatic anion.

3. The composition of any of the preceding claims, wherein the fatty acid comprises an aliphatic chain of no more than 3 carbons.

4. The composition of any of the preceding claims, wherein the anion comprises only one carboxylic acid group (e.g., R—COOH group).

5. The composition of any of the preceding claims, wherein the anion is selected from the group consisting of:
geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic

6. The composition of any of the preceding claims, wherein the anion is selected from the group consisting of:
glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid.

7. The composition of any of the preceding claims, wherein the cation has a molar mass equal to or greater than choline.

8. The composition of any of the preceding claims, wherein the quaternary ammonium has the structure of NR₄⁺ and at least one R group comprises a hydroxy group.

9. The composition of any of the preceding claims, wherein the quaternary ammonium has the structure of NR₄⁺ and only one R group comprises a hydroxy group.

10. The composition of any of the preceding claims, wherein the cation is choline, C1, C6, or C7.

11. The composition of any of the preceding claims, wherein the cation is choline.

12. The composition of any of the preceding claims, wherein the cation is C1, C6, or C7.

13. The composition of any of the preceding claims, wherein the ionic liquid comprises a ratio of cation to anion of from about 2:1 to about 1:1.

14. The composition of any of the preceding claims, wherein the ionic liquid comprises a ratio of cation to anion of about 2:1.

15. The composition of any of the preceding claims, wherein the ionic liquid has a cation:anion ratio of less than 1:1.

16. The composition of any of the preceding claims, wherein the ionic liquid has a cation:anion ratio with an excess of cation.

17. The composition of any of the preceding claims, comprising a first ionic liquid and at least a second ionic liquid.

18. The composition of claim 17, wherein each ionic liquid has a choline cation.

19. The composition of any of claims 17-18, wherein the first ionic liquid and the second ionic liquid each comprise a different anion.

20. The composition of claim 19, wherein the first ionic liquid and the second ionic liquid each comprise a different anion selected from:
geranic acid; glycolic acid; propanoic acid; isobutyric acid; butyric acid; gallic acid; lactic acid; malonic acid; maleic acid; glutaric acid; citric acid; 3,3-dimethylacrylic acid; dimethylacrylic acid; gluconic acid; adipic acid; sodium ethylhexyl sulfate; decanoic acid; hydroxybenzenesulfonic acid; 4-hydroxybenzenesulfonic acid (4-phenolsulfonic acid); isovaleric acid; hydrocinnaminic acid (phenylpropanoic acid); phenyl phosphoric acid; and biphenyl-3-carboxylic acid.

21. The composition of any of claims 17-20, wherein the first ionic liquid has a geranic acid anion and the second ionic liquid has a phenylpropanoic acid anion.

22. The composition of any of claims 17-21, wherein the first ionic liquid is choline and geranic acid (CAGE).

23. The composition of any of claims 17-22, wherein the second ionic liquid is choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).

24. The composition of any of claims 17-21, wherein the first and second ionic liquids are different ionic liquids selected from the group consisting of: choline and geranic acid (CAGE); choline and dimethylacrylic acid (CADA); choline and isovaleric acid (CAVA); choline and phenylphosphoric acid (CAPP); choline and biphenyl-3-carboxylic acid (CABA); choline and 4-phenolsulfonic acid (CASA); or choline and phenylpropanoic acid (CAPA).

25. The composition of any of claims 17-21, wherein the first ionic liquid is selected from the group consisting of: choline and geranic acid (CAGE); choline and dimethylacrylic acid (CADA); and choline and choline and biphenyl-3-carboxylic acid (CABA); and
the second ionic liquid is selected from the group consisting of: isovaleric acid (CAVA); and choline and phenylpropanoic acid (CAPA).

26. The composition of any of claims 17-22, wherein the first ionic liquid is choline and geranic acid (CAGE) and the second ionic liquid is choline and phenylpropanoic acid (CAPA).

27. The composition of any of the preceding claims, further comprising at least one active compound in combination with the at least one ionic liquid.

28. The composition of any of the preceding claims, wherein the active compound comprises a polypeptide.

29. The composition of claim 28, wherein the polypeptide is an antibody or antibody reagent.

30. The composition of any of claims 28-29, wherein the active compound has a molecular weight of greater than 450.

31. The composition of any of claims 28-30, wherein the active compound has a molecular weight of greater than 500.

32. The composition of any of claims 28-31, wherein the anion has a LogP of less than 1.0 and is:
a. a carboxylic acid which is not a fatty acid; or
b. a carboxylic acid comprising an aliphatic chain of no more than 4 carbons.

33. The composition of any of the preceding claims, wherein the active compound comprises a nucleic acid.

34. The composition of claim 33, wherein the nucleic acid is an inhibitory nucleic acid.

35. The composition of claim 34, wherein the nucleic acid is a siRNA.

36. The composition of any of claims 34-35, wherein the inhibitory nucleic acid is a NFKBIZ, TNFalpha, and/or IL-17 inhibitory nucleic acid.

37. The composition of any of claims 33-36, wherein the anion has a LogP of less than 1.0 and is:
a. a carboxylic acid which is not a fatty acid; or
b. a carboxylic acid comprising an aliphatic chain of no more than 4 carbons; and/or
c. an aromatic anion.

38. The composition of any of the preceding claims, wherein the ionic liquid is at a concentration of at least 0.1% w/v.

39. The composition of any of the preceding claims, wherein the ionic liquid is at a concentration of from about 10 to about 70% w/v.

40. The composition of any of the preceding claims, wherein the ionic liquid is at a concentration of from about 30 to about 50% w/v.

41. The composition of any of the preceding claims, wherein the ionic liquid is at a concentration of from about 30 to about 40% w/v.

42. The composition of any of the preceding claims, wherein the composition is formulated for administration transdermally, to a mucus membrane, orally, subcutaneously, intradermally, parenterally, intratumorally, or intravenously.

43. The composition of claim 42, wherein the composition is formulated for transdermal administration.

44. The composition of claim 42, wherein the mucus membrane is nasal, oral, or vaginal.

45. The composition of any of the preceding claims, wherein the active compound is provided at a dosage of 1-40 mg/kg.

46. The composition of any of the preceding claims, further comprising at least one non-ionic surfactant.

47. The composition of any of the preceding claims, further comprising a pharmaceutically acceptable carrier.

48. The composition of any of the preceding claims, wherein the composition is provided in a degradable capsule.

49. The composition of any of the preceding claims, wherein the composition is an admixture.

50. The composition of any of the preceding claims, wherein the composition is provided in one or more nanoparticles.

51. The composition of any of the preceding claims, comprising one or more nanoparticles comprising the active compound, the nanoparticles in solution or suspension in a composition comprising the ionic liquid.

52. A method of administering at least one active compound to a subject, the method comprising administering a composition of any of claims 27-51.

53. The method of claim 52, wherein the composition is administered once.

54. The method of any of claims 52-53, wherein the composition is administered in multiple doses.

55. The method of any of claims 52-54, wherein the administering is transdermally, to a mucus membrane, orally, subcutaneously, intradermally, parenterally, intratumorally, or intravenously

56. The method of any of claims 52-55, wherein the composition comprises a NFKBIZ, TNFalpha, and/or IL-17 inhibitory nucleic acid and the subject is in need of treatment for an inflammatory condition.

57. A method of treating an inflammatory condition in a subject in need thereof, the method comprising administering a composition of any of claims 36-51 to the subject.

58. The method of any of claims 56-57, wherein the administration is topical.

59. The method of any of claims 56-58, wherein the inflammatory condition is psoriasis.

60. A composition of any of claims 27-51, for use in a method of administering at least one active compound to a subject.

61. The composition of claim 60, wherein the composition is administered once.

62. The composition of claim 60, wherein the composition is administered in multiple doses.

63. The composition of any of claims 60-62, wherein the administering is transdermally, to a mucus membrane, orally, subcutaneously, intradermally, parenterally, intratumorally, or intravenously

64. The composition of any of claims 60-63, wherein the composition comprises a NFKBIZ, TNFalpha, and/or IL-17 inhibitory nucleic acid and the subject is in need of treatment for an inflammatory condition.

65. A composition of any of claims 36-51 for use in a method of treating an inflammatory condition in a subject in need thereof.

66. The composition of any of claims 64-65, wherein the administration is topical.

67. The composition of any of claims 64-66, wherein the inflammatory condition is psoriasis.


SEQUENCE LISTING (40 sequences)

SEQ ID NO: 1 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 2 - protein, 37 aa, Homo sapiens
HDEFERHAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 31 aa, Homo sapiens
HAEGTFTSDVSSYLEGQAAKEFIAWLVKGRG

SEQ ID NO: 5 - RNA, 21 nt, Artificial Sequence
  source: /note=“Description of Artificial Sequence Synthetic oligonucleotide”
gugugaaccacgagaaauauu

SEQ ID NO: 6 - RNA, 21 nt, Artificial Sequence
  source: /note=“Description of Artificial Sequence Synthetic oligonucleotide”
aauauuucucgugguucacac

SEQ ID NO: 7 - DNA, 20 bp, Artificial Sequence
  source: /note=“Description of Artificial Sequence Synthetic primer”
accacagtccatgccatcac

SEQ ID NO: 8 - DNA, 20 bp, Artificial Sequence
  source: /note=“Description of Artificial Sequence Synthetic primer”
tccaccaccctgttgctgta

SEQ ID NO: 9 - DNA, 20 bp, Artificial Sequence
  source: /note=“Description of Artificial Sequence Synthetic primer”
tatcgggtgacacagttgga

SEQ ID NO: 10 - DNA, 20 bp, Artificial Sequence
  source: /note=“Description of Artificial Sequence Synthetic primer”
tgaatggacttccccttcag

SEQ ID NO: 11 - DNA, 21 bp, Artificial Sequence
  source: /note=“Description of Artificial Sequence Synthetic primer”
ggcaggttctgtccctttcac

SEQ ID NO: 12 - DNA, 24 bp, Artificial Sequence
  source: /note=“Description of Artificial Sequence Synthetic primer”
ttctgtgctcatggtgtcttttct

SEQ ID NO: 13 - DNA, 20 bp, Artificial Sequence
  source: /note=“Description of Artificial Sequence Synthetic primer”
atgagtgccgacaaacaacg

SEQ ID NO: 14 - DNA, 20 bp, Artificial Sequence
  source: /note=“Description of Artificial Sequence Synthetic primer”
gtgacgtggaacggttgagg

SEQ ID NO: 15 - DNA, 20 bp, Artificial Sequence
  source: /note=“Description of Artificial Sequence Synthetic primer”
ctggaagctgacactcacga

SEQ ID NO: 16 - DNA, 20 bp, Artificial Sequence
  source: /note=“Description of Artificial Sequence Synthetic primer”
ggtagcggttctcatctgtg

SEQ ID NO: 17 - DNA, 20 bp, Artificial Sequence
  source: /note=“Description of Artificial Sequence Synthetic primer”
ttccacgagatcaagagagc

SEQ ID NO: 18 - DNA, 20 bp, Artificial Sequence
  source: /note=“Description of Artificial Sequence Synthetic primer”
tctacacctgttccgctgag

SEQ ID NO: 19 - DNA, 22 bp, Artificial Sequence
  source: /note=“Description of Artificial Sequence Synthetic primer”
ttgaggtgtccaacttccagca

SEQ ID NO: 20 - DNA, 20 bp, Artificial Sequence
  source: /note=“Description of Artificial Sequence Synthetic primer”
gcataggtagccagagccag

SEQ ID NO: 21 - DNA, 21 bp, Artificial Sequence
  source: /note=“Description of Artificial Sequence Synthetic primer”
tggcatcgagaaactgtgaga

SEQ ID NO: 22 - DNA, 25 bp, Artificial Sequence
  source: /note=“Description of Artificial Sequence Synthetic primer”
tcagttcgtattggtagtcctgtta

SEQ ID NO: 23 - DNA, 24 bp, Artificial Sequence
  source: /note=“Description of Artificial Sequence Synthetic primer”
agtgggtgtagttctgtagtgtgc

SEQ ID NO: 24 - DNA, 25 bp, Artificial Sequence
  source: /note=“Description of Artificial Sequence Synthetic primer”
gttcgtctcaagagtgtccagatat

SEQ ID NO: 25 - DNA, 21 bp, Artificial Sequence
  source: /note=“Description of Artificial Sequence Synthetic primer”
cacagatgagaaccgctaccc

SEQ ID NO: 26 - DNA, 21 bp, Artificial Sequence
  source: /note=“Description of Artificial Sequence Synthetic primer”
gcggatgaactcggtgtggaa

SEQ ID NO: 27 - DNA, 21 bp, Artificial Sequence
  source: /note=“Description of Artificial Sequence Synthetic primer”
gtgggtttcacaagacagatg

SEQ ID NO: 28 - DNA, 21 bp, Artificial Sequence
  source: /note=“Description of Artificial Sequence Synthetic primer”
ttttcacccagttctgctttg

SEQ ID NO: 29 - DNA, 20 bp, Artificial Sequence
  source: /note=“Description of Artificial Sequence Synthetic primer”
ccttctcagatggagcgcag

SEQ ID NO: 30 - DNA, 20 bp, Artificial Sequence
  source: /note=“Description of Artificial Sequence Synthetic primer”
tgtccaggtcctccatgatg

SEQ ID NO: 31 - DNA, 20 bp, Artificial Sequence
  source: /note=“Description of Artificial Sequence Synthetic primer”
ggaccagggctgtcgctact

SEQ ID NO: 32 - DNA, 20 bp, Artificial Sequence
  source: /note=“Description of Artificial Sequence Synthetic primer”
ggatcccgatggctagagca

SEQ ID NO: 33 - DNA, 24 bp, Artificial Sequence
  source: /note=“Description of Artificial Sequence Synthetic primer”
agggaaggatgagattaagactgg

SEQ ID NO: 34 - DNA, 21 bp, Artificial Sequence
  source: /note=“Description of Artificial Sequence Synthetic primer”
cttgctggttcttcgtctttt

SEQ ID NO: 35 - DNA, 24 bp, Artificial Sequence
  source: /note=“Description of Artificial Sequence Synthetic primer”
cggttccgatgccctgaggctctt

SEQ ID NO: 36 - DNA, 24 bp, Artificial Sequence
  source: /note=“Description of Artificial Sequence Synthetic primer”
cgtcacacttcatgatggaattga

SEQ ID NO: 37 - DNA, 3790 bp, Homo sapiens
ctcctcttgccacgaggtcagacggcgagttcttagagaaaaaggctgcttagctgctgcttatcatgtaacctcaaaaggaaactgatcgtctttctcatgctgtcacgtacttgggttattatcgctgattacagctggaaacaattgatttgctcttacgtatttgtgtgacttgactcttcaaacacaaaggttaacaggaagatctcgagggccctggctgaacttcaccttttggctttcttggcctgatgctgaactctcgaggttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaata

SEQ ID NO: 38 - DNA, 3923 bp, Homo sapiens
gtactggcccgcgccgtccgcccgccgacagctccctgagccagcccgggaggcagccgcgcgcagcgagccggtggcgcaggtgtcggggtcctcgagcgcccagcctgggagcatgattgtggacaagctgctggacgacagccgcggcggagaggggctgcgggacgcggcgggcggctgcggcctcatgaccagcccgctcaacctgagctacttctacggcgcgtcgccgcccgccgccgccccgggcgcctgcgacgccagctgctcggtcttgggcccctcggcgcccggctcgcccggctccgactcctccgacttctcctctgcctcgtcggtgtcctcctgcggcgccgtggagtcccggtcgagaggcggcgcccgcgccgagcgccagccagttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaata

SEQ ID NO: 39 - DNA, 1678 bp, Homo sapiens
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 40 - DNA, 1871 bp, Homo sapiens
gtccatctcatagcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc